(19) United States
(12) Patent Application Publication
(10) Pub. No.: US 2016/0108468 A1
(43) Pub. Date: Apr. 21, 2016

(54) Isothermal Amplification of Nucleic Acid, and Library Preparation and Clone Generation in Sequencing

(71) Applicant: Ohio State Innovation Foundation, Columbus, OH (US)

(72) Inventor: Besik KANKIA, Columbus, OH (US)

(21) Appl. No.: 14/772,568

(22) PCT Filed: Mar. 6, 2014
(86) PCT No.: PCT/US2014/021165

§ 371 Date: Sep. 3, 2015

Related U.S. Application Data
(60) Provisional application No. 61/818,490, filed on May 2, 2013.
(60) Provisional application No. 61/773,430, filed on Mar. 6, 2013.

(51) Int. Cl. C12Q 1/68

(52) U.S. Cl. CPC C12Q 1/6839; C12Q 1/6806; C12Q 1/6874; C12Q 1/686; C12Q 1/6818

(57) ABSTRACT

An amplification system that provides methods and reaction components that allow for isothermal amplification for detection of target nucleic acid 24; allow non-enzymatic amplification for detection of target nucleic acid 24; can be used to identify amplicons without having to create separate individual probes for each target nucleic acid 24, and can be used to improve sequencing processes.

### CROSS-REFERENCE TO RELATED APPLICATIONS

This application claims priority to and the benefit of the filing dates of U.S. Patent Application Ser. No. 61/773,430, entitled “Two-primer, Exponential and Isothermal QPA,” filed on Mar. 6, 2013, and U.S. Patent Application Ser. No. 61/818,490, entitled “Quadruplex-Based Technology for Mono-Adapter DNA Library Preparation and Isothermal Clone Generation for Next Generation Sequencing (NGS),” filed on May 2, 2013, the disclosures of which are incorporated by reference herein in their entireties.

### FIELD OF THE INVENTION

The present invention relates generally to detection and/or identification of nucleic acids via an amplification process, and more specifically to novel methods and components for detection and/or identification of nucleic acids via an amplification process. The present invention also relates generally to the preparation of libraries of nucleic acids and the generation of clones for next generation sequencing (“NGS”), and more specifically to novel methods and components for same.

### BACKGROUND

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

Identification of a particular nucleic acid (e.g., via detection of the presence of a particular nucleic acid sequence—the “target nucleic acid”) is desirable for many reasons, including use in diagnostic applications, forensic applications, etc. However, often the target nucleic acid sequence may be only a small portion of the DNA or RNA in question, and/or the quantity of DNA or RNA may be limited so that it may be difficult to detect the presence of the target sequence using probes (such as oligonucleotide probes). Much effort has been expended in increasing the sensitivity of the probe detection systems, and processes have also been developed for amplifying the target sequence so that it is present in quantities sufficient to be readily detectable.

One such amplification method is the polymerase chain reaction (PCR). Since its initial design by Kary Mullis in 1984, PCR has impacted nearly every field in molecular biology, genetics, and forensic science. PCR is a technique to amplify a single or few copies of a particular nucleic acid sequence, e.g., DNA, across several orders of magnitude, thereby generating thousands to millions of copies of the sequence. PCR is the main tool presently used to amplify nucleic acid and study gene expression.

PCR relies on “thermal cycling,” which includes cycles of repeated heating and cooling of the DNA and other reaction components to cause DNA denaturation (i.e., separation of the double-stranded DNA into its sense and antisense strands) followed by enzymatic replication of the DNA. The other reaction components include short oligonucleotide DNA fragments known as “primers,” which contain sequences complementary to at least a portion of the DNA sequence associated with the target nucleic acid, and a DNA polymerase. These are components that facilitate selective and repeated amplification of the target sequence. As PCR progresses, the DNA generated is itself used as a template for further replication in subsequent cycles, creating a chain reaction in which the target DNA sequence is exponentially amplified.

More specifically, the DNA polymerase used in PCR is thermostable (and thus avoids enzyme denaturation at high temperatures) and amplifies target DNA by in vitro enzymatic replication. One such thermostable DNA polymerase is Taq polymerase, an enzyme originally isolated from the bacterium Thermus aquaticus. The DNA polymerase enzymatically assembles a new DNA strand from deoxynucleoside triphosphates (dNTPs) by using the denatured single-stranded DNA as a template. As is known to those of ordinary skill in the art, a deoxynucleoside triphosphate is deoxyribose having three phosphate groups attached, and having one base (adenine, guanine, cytosine, thymine) attached. However, as used herein, it will be recognized by those of ordinary skill in the art that arsenic may be substituted for phosphorous in the triphosphate backbone of the dNTP. The initiation of DNA synthesis and the selectivity of PCR results from the use of primers that are complementary to the DNA region targeted for amplification under specific thermal cycling conditions.

Thus, a basic PCR set up includes multiple components. These include: (1) a DNA template that contains the target DNA region to be amplified; (2) primers that are complementary to the 3′ ends of each of the sense strand and antisense strand of the target DNA; (3) a thermostable DNA polymerase such as Taq polymerase; and (4) dNTPs, the building blocks from which the DNA polymerases synthesizes a new DNA strand. Additionally, the reaction will generally include other components such as a buffer solution providing a suitable chemical environment for optimum activity and stability of the DNA polymerase, divalent cations (generally magnesium ions), and monovalent cation potassium ions (K⁺).

PCR is commonly carried out in a reaction volume of 10-200 μl in small reaction tubes (0.2-0.5 ml volumes) in an apparatus referred to as a thermal cycler. The thermal cycler heats and cools the reaction tubes to achieve the temperatures required at each of the following steps of the reaction:

Denaturation Step:

This step consists of heating the reaction to usually around 94-98° C. for approximately 20-30 seconds. It causes denaturation of the DNA template by disrupting the hydrogen bonds between complementary bases, yielding single strands of DNA.

Annealing Step:

The reaction temperature is lowered to usually around 50-65° C. for approximately 20-40 seconds allowing annealing of the primers to the single-stranded DNA template. Stable DNA-DNA hydrogen bonds are formed when the primer sequence closely matches the template sequence. The polymerase (e.g., Taq polymerase) binds to the primer-template hybrid and begins DNA synthesis.

Extension Step:

The temperature at this step depends on the DNA polymerase used. Taq polymerase has its optimum activity temperature at about 75° C., and commonly a temperature of 72° C. is used with this enzyme. At this step the DNA polymerase synthesizes a new DNA strand complementary to the DNA template strand by adding dNTPs that are complementary to the template in the 5′ to 3′ direction, condensing the 5′-phosphate group of the dNTPs with the 3′-hydroxyl group at the end of the extending DNA strand (as described above arsenic may substitute for phosphorous in a dNTP). The extension time depends on the DNA polymerase used and on the length of the DNA fragment to be amplified. Under optimum conditions, at each extension step the amount of the target DNA is doubled, leading to exponential amplification of the specific target DNA.

PCR usually includes of a series of 20 to 40 repeated cycles of the above-described denaturation, annealing, and extension steps. The cycling is often preceded by a single initialization step at a high temperature (>90° C.), and followed by one final hold at the end for final product extension or brief storage. The initialization step consists of heating the reaction to a temperature of usually 94-96° C. (or 98° C. if extremely thermostable polymerases are used), which is held for 1-9 minutes. The final hold usually occurs at 4-15° C. for an indefinite time and may be employed for short-term storage of the reaction. The temperatures used and the length of time they are applied in each cycle depend on a variety of parameters. These include the enzyme used for DNA synthesis, the concentration of divalent ions and dNTPs in the reaction, and the melting temperature (Tₘ) of the primers.

Following thermal cycling, agarose gel electrophoresis may be employed for size separation of the PCR products to check whether PCR amplified the target DNA fragment. The size(s) of the PCR products is determined by comparison with a molecular weight marker, which contains DNA fragments of known size, run on the gel alongside the PCR products. Probes may also be used to identify the presence of an amplified target DNA fragment (e.g., an oligonucleotide probe having a detectable label and a sequence complementary to the target nucleic acid sequence may be used; the probe will hybridize to target nucleic acid that is present and the label can be detected, thereby signifying the presence of the target nucleic acid).

There are many applications of PCR and its variants. For example, real-time PCR (RT-PCR) is an established tool for DNA quantification that measures the accumulation of DNA product after each round of PCR amplification. Thus, RT-PCR enables both detection and quantification of one or more specific sequences in a DNA sample (as absolute number of copies or relative amount when normalized to DNA input or additional normalizing genes). Such quantitative PCR methods allow the estimation of the amount of a given sequence present in a sample—a technique often applied to quantitatively determine levels of gene expression.

RT-PCR procedure follows the general principle of PCR. However, in RT-PCR, the amplified DNA is detected as the reaction progresses in real time (whereas in standard PCR, the product of the reaction is detected at the end of the reaction). One common method for detection of products in RT-PCR is the use of nonspecific fluorescent dyes that intercalate with double-stranded DNA (dsDNA). For example, SYBR Green is an asymmetrical cyanine dye that binds to dsDNA, and the resulting DNA-dye complex absorbs blue light (λₘₐₓ=488 nm) and emits green light (λₘₐₓ=522 nm). The DNA-binding dye, such as SYBR Green, binds to all dsDNA in PCR, causing fluorescence of the dye. An increase in DNA product during PCR therefore leads to an increase in fluorescence intensity and is measured at each cycle, thus allowing DNA concentrations to be quantified.

Another method for detection of products in RT-PCR is the use of sequence-specific DNA probes, which are oligonucleotides that are labeled with a fluorescent reporter that permits detection after hybridization of the probe with its complementary DNA target. Many of these probes include a DNA-based probe having a fluorescent reporter (e.g., at one end of the probe) and a quencher of fluorescence (e.g., at the opposite end of the probe). The close proximity of the reporter to the quencher prevents detection of its fluorescence; breakdown of the probe by the 5′ to 3′ exonuclease activity of the Taq polymerase breaks the reporter-quencher proximity and thus allows unquenched emission of fluorescence, which can be detected. An increase in the product targeted by the reporter probe at each PCR cycle therefore causes a proportional increase in fluorescence due to the breakdown of the probe and release of the reporter. Examples of such probes well known to those of ordinary skill in the art are molecular beacon probes, TaqMan® probes, and Scorpion™ probes.

Molecular beacons are single-stranded oligonucleotide probes that form a hairpin-shaped stem-loop structure. The loop contains a probe sequence that is complementary to a target sequence in the PCR product. The stem is formed by the annealing of complementary sequences that are located on either side of the probe sequence. A fluorophore and quencher are covalently linked to the ends of the hairpin. Upon hybridization to a target sequence the fluorophore is separated from the quencher and fluorescence increases. Hybridization usually occurs after unfolding of the hairpin and product duplexes in the denaturation step of the next PCR cycle.

TaqMan® probes are single-stranded unstructured oligonucleotides. They have a fluorophore attached to the 5′ end and a quencher attached to the 3′ end. When the probes are free in solution, or hybridized to a target, the proximity of the fluorophore and quencher molecules quenches the fluorescence. During PCR, when the polymerase replicates a template on which a TaqMan® probe is bound, the 5′-nuclease activity of the polymerase cleaves the probe. Upon cleavage, the fluorophore is released and fluorescence increases.

Scorpion™ probes use a single oligonucleotide that consists of a hybridization probe (stem-loop structure similar to molecular beacons) and a primer linked together via a non-amplifiable monomer. The hairpin loop contains a specific sequence that is complementary to the extension product of the primer. After extension of the primer during the extension step of a PCR cycle, the specific probe sequence is able to hybridize to its complement within the extended portion when the complementary strands are separated during the denaturation step of the subsequent PCR cycle, and fluorescence will thus be increased (in the same manner as molecular beacons).

With the above general background in mind, there are drawbacks to current PCR processes and probe technologies. For example, one of the factors limiting the yield of specific target nucleic acid is competition between primer binding and self-annealing of the product. At the initial stage of PCR, target molecules are at low enough concentrations that target self-annealing does not compete with primer binding and amplification thus proceeds at an exponential rate. However, with accumulation of target nucleic acid (e.g., DNA), self-annealing becomes dominant and PCR slows down and eventually amplification ceases.

Temperature cycling is another limitation of PCR since it requires expensive instrumentation for thermocycling, thereby complicating rapid detection of pathogens in the field and at point-of-care. In addition, rapid temperature changes facilitate mispriming of the target nucleic acid and affect stability of the polymerases.

To eliminate problems caused by temperature cycling, expensive instrumentation, competition between primer binding and undesired self-annealing of target DNA, and difficulty with point-of-care testing, further amplification methods and components (e.g., primers) have been recently developed (as described in U.S. Provisional Application Ser. No. 61/338,475 and International Application No. PCT/US2011/25411, the disclosures of both of which are incorporated herein in their entireties). These methods and components, in general, inhibit self-annealing by providing at least one primer (e.g., an oligonucleotide) for amplification of a target nucleic acid (e.g., DNA), wherein the primer is adapted to conform into a structure (e.g., a non-B DNA conformation or other DNA structure) in which intramolecular base-pairing allows or causes the primer to dissociate from a double-stranded DNA (which may be referred to herein as a “dissociative structure” or “dissociative sequence”). Such a structure may include triplexes or quadruplexes. The particular structure—e.g., a quadruplex—may form during an extension step of an amplification method, such as PCR. As this occurs, the primer necessarily separates from its binding site on the target DNA sequence while the extending portion (DNA polymerase adding dNTPs to the sequence) remains bound (at least temporarily) in a double-stranded configuration. This process may be referred to herein as “Dissociative Sequence Priming Amplification” or “Dissociative Structure Priming Amplification” (which may be referred to herein as “DSPA”). Other amplification processes may be compatible with DSPA. For example, helicase-dependent amplification (wherein a helicase is used to unwind the DNA duplex without having to alter the temperature of the reaction) may be compatible with DSPA (e.g., helicases may be used in the DSPA process).

By use of primers that form dissociative structures, an amplification process that is primarily isothermal is achieved. In other words, the process of amplification, once begun with DSPA primers, can be isothermal. This is because, as described above, when the primer forms its dissociative structure, it necessarily separates from its binding site on the target DNA sequence, and so this is achieved without having to raise the temperature of the reaction to denature the strands. However, as will be appreciated by those of ordinary skill in the art, naturally-occurring target DNA will not necessarily include a sequence complementary to the DSPA primer sequence (where the DSPA primer can bind to begin replication). And so, the DNA templates in these reactions must first have primer binding site (PBS) sequences added to the DNA template. This can be accomplished by 2 cycles of traditional PCR. Once accomplished, the DSPA primer(s) [such as quadruplex forming primer(s)] will be added to the mix and amplification may be continued under isothermal conditions. However, the use of these 2 cycles of traditional PCR prevents the method from being completely isothermal, and prevents the complete elimination of thermal cycling, equipment, etc., which is a barrier to point-of-care use.

Several other versions of isothermal DNA amplification have previously been developed [see Tomita, N. et al. (2008) Loop-mediated isothermal amplification (LAMP) of gene sequences and simple visual detection of products. Nature protocols, 3, 877-882; Vincent, M., et al. (2004) Helicase-dependent isothermal DNA amplification. EMBO reports, 5, 795-800; Andras, S. C. et al. (2001) Strategies for signal amplification in nucleic acid detection. Molecular biotechnology, 19, 29-44; Walker, G. T. et al. (1992) Strand displacement amplification—an isothermal, in vitro DNA amplification technique. Nucleic acids research, 20, 1691-1696; Walker, G. T. et al. (1992) Isothermal in vitro amplification of DNA by a restriction enzyme/DNA polymerase system. Proceedings of the National Academy of Sciences of the United States of America, 89, 392-396; Fox, J. D. et al. (2009) In Logan, J. et al. (eds.), Real-time PCR. Caister Academic Press, Norfolk, UK, pp. 163-175]. However, all these existing systems require extra reaction components or polymerases with specific activities, presenting drawbacks to these systems, as well.

In addition to the problems with developing a completely isothermal amplification process, there are other drawbacks to current PCR processes. For example, traditional PCR depends on the enzymatic activity of DNA polymerases. Such polymerization-based amplification can yield a macroscopically observable polymer, visible to the unaided eye, which is a preferable quality for point-of-care analysis. However, it requires coupling biotinylated dNTPs to DNA hybrids and requires 0.5 nM or higher concentrations of target nucleic acid, which reduces the usefulness of the test. Surface plasmon resonance coupled with interferometry can detect as little as 10 μM of target (thereby resolving the initial problem), but this requires instrumentation unsuitable for point-of-care diagnostics (thereby creating another problem). Gold nanoparticles can be visualized with the unaided eye at high pM to nM target concentrations. However, to increase sensitivity further, they must be coupled with PCR or other specialized detection platforms. A sandwich-type binding assay is able to detect 60 fmol target DNA, however it depends on biotinylated capture oligonucleotides, repeated washing steps and additional liposome components. Hybridization chain reaction and entropy-driven signal amplification do not require the use of specific detection platforms, and are based on autocatalytic reactions between DNA oligonucleotides in solution. However, both of those methods use complicated reactions and detection mechanisms. In addition, these methods suffer from low sensitivity due to significant levels of spontaneous autocatalysis even in the absence of target molecules. As a result, all of the above-described methods and variations are not suitable for point-of-care analysis.

Still further, there are drawbacks to the probes used to detect products in RT-PCR, such as sequence-specific DNA probes (e.g., molecular beacons, TaqMan®, and Scorpion™ probes). For example, there are several disadvantages with molecular beacons. First, they require two bulky and costly tags (fluorophore and quencher). Second, the assay requires a separate probe for each template (i.e., mRNA), which dramatically increases the design effort and expense. Third, the mechanism uses separate binding sites for primer and probe sequences, which introduces another component (probe oligonucleotide) to an already complex reaction, and adds additional design limitations due to the need to avoid interactions between the probe and primers. Fourth, hybridization of the probe requires heating steps to unfold the product duplex and hairpin. Consequently, molecular beacons can't be used under isothermal conditions. Fifth, design of the probe requires considerable effort and knowledge of nucleic acid thermodynamics. And sixth, probe hybridization involves a bimolecular probe-primer system. This makes the reaction entropically unfavorable, slows down hybridization, and complicates product detection at exponential growth. The hybridization is much faster and efficient with a monomolecular probe-primer system [as described in Whitcombe, D. et al. (1999) Detection of PCR products using self-probing amplicons and fluorescence. Nat Biotechnol, 17, 804-807, incorporated by reference herein in its entirety].

All of the shortcomings listed above for molecular beacons hold true for TaqMan® probes. And, an additional disadvantage of TaqMan® probes is that they require the 5′-nuclease activity of the DNA polymerase used for PCR.

Additionally, many of the shortcomings listed for molecular beacons hold true for Scorpion™ probes. First, they require two bulky and costly tags (fluorophore and quencher). Second, the assay requires a separate probe for each template (i.e., mRNA), which dramatically increases the design effort and expense. Third, the mechanism uses separate binding sites for primer and probe sequences, which introduces another component (probe oligonucleotide) to an already complex reaction, and adds additional design limitations due to the need to avoid interactions between the probe and primers. Fourth, hybridization of the probe requires heating steps to unfold the product duplex and hairpin. Consequently, Scorpion™ probes can't be used under isothermal conditions. And fifth, design of the probes requires considerable effort and knowledge of nucleic acid thermodynamics.

Further, fluorescent reporter probes do not prevent the inhibitory effect of primer dimers (i.e., sets primers that are complementary to one another, and thus hybridize to one another—forming a primer dimer—rather than hybridizing to the target template denatured DNA strands), which may depress accumulation of the desired products in the reaction.

Still another disadvantage of current detection mechanisms is that two separate functions, recognition and detection, are combined within a probe. For example, the traditional RT-PCR process includes: (i) recognition of target nucleic acid by primer(s); (ii) subsequent amplification; and then (iii) recognition of amplicons by a probe, which is accompanied by fluorescence reporting. Thus, in traditional RT-PCR recognition happens twice (primer recognition and probe recognition). The bifunctional nature of the probes (i.e., the probes provide both recognition and reporting) requires that the fluorophore-quencher pair be attached to each DNA probe sequence, which makes quantification impractical when several targets are tested. For example, to perform 96-well quantification using molecular beacons, it is necessary that the same fluorophore-quencher pair be attached to 96 different probes. This greatly increases the time, difficulty, and expense of such a test.

Apart from prior and currently used processes for amplification of nucleic acids—such as PCR—similar drawbacks to those described above present themselves in the areas of preparation of DNA libraries and clone generation for sequencing, such as in next generation sequencing (NGS).

As is generally known to those of ordinary skill in the art, library preparation for the major next generation sequencing platforms requires the ligation of specific adaptor oligos to fragments of the DNA to be sequenced.

The starting material for library construction is double stranded (ds) DNA from any source: including, but not limited to, genomic DNA, BACs, PCR amplicons, ChIP samples, or any type of RNA turned into ds DNA (mRNA, total RNA, smRNAs, etc). New protocols for library preparation are frequently generated, so the following is illustrative of the typical steps involved in library preparation techniques and is representative of the processes that have been and continue to be used to generate many sequencing libraries. A brief description of the steps involved in library generation follows.

Fragmentation of the Starting Material:

Initially, the starting material is fragmented. For high MW DNA, fragmentation is typically accomplished by sonication or nebulization. Other options for fragmentation include enzymatic fragmentation, or micrococcal nuclease digestion. Most RNA-seq protocols include a fragmentation step as part of the conversion to cDNA, so additional sonication is not necessary for these libraries. Similarly, chromatin immunoprecipitated DNA already has been sonicated, so additional steps to reduce the MW of the material for sequencing is usually not necessary.

End Repair/A-Tailing:

Following fragmentation, the ends of the DNA must be “polished” so that an A-tail facilitating downstream ligation steps can be added. End repair enzymes are generally included in library preparation kits from most manufacturers.

Adapter Ligation:

In this step T-tailed adapter molecules containing functional sequences used in library amplification and sequencing are ligated to the fragmented DNA of interest. Similarly to the other steps, either stand alone kits just for this step (lately we have been using ligase purchased directly from Enzymatics) or use of materials found in library construction kits achieve similar results. Adapters can generally be commercially obtained in ready-to-go format, or they can be synthesized as single strand oligos then annealed and used.

One drawback that presents itself at this step is a result of the molar ratio between adapters and DNA insert. Adapters generally come at, or are prepared to, a concentration of 50-100 uM. However, if too much adapter is present, adapters will ligate to other adapters (despite the T-overhang, which is added to help reduce this self ligation), and these will be preferentially amplified in the PCR step (described below). If too little adapter is present, chimeric inserts can form, or insert sequences are left behind as they search fruitlessly for a partner. Operationally speaking, it is difficult to quantify the amount of insert.

Thus, with techniques for library preparation, the random attachment of two different adapters (A and B) to DNA fragments, employed in all NGS, is a very inefficient way of library preparation. First, due to producing homo-adapter fragments (A-A and B-B instead of A-B)—as described above—a significant amount of DNA fragments is wasted, which necessitates a large amount of genomic DNA and multiple sequencing reactions. Second, the library enrichment process (e.g., cleaning from homo-adapter fragments to avoid further complications and expenses) significantly elongates workflow. Third, enriched DNA fragments can be sequenced only in one direction (complementary strands are discarded).

PCR Amplification:

The adapter-ligation reaction is then amplified using standard PCR techniques (such as those described above) to produce the final product for cluster formation and sequencing. Much has been written about PCR: biases introduced by the process, which enzymes to use, what sequence modifications can be added at this step versus ligation, and other topics have been extensively covered. It is important not to over-amplify. Too many cycles can generate artefactual duplicates as well as higher MW library isoforms of indeterminate structure. But over amplification can be difficult to assess before the fact, and depends on a number of items like volume and concentration of the adapter ligation mix, how this mix was generated (ie, has it been size selected, and if so, how, was it from a library prep robot or another custom method, etc.), and what kind of library is being produced. Since it is possible to quantify and analyze very low amounts of library, it is generally thought better to be conservative with cycle number.

Clone Generation in NGS:

The sequencing process in NGS is massively parallel, which requires DNA clusters immobilized at solid support. After (i) breaking genomic DNA into smaller sizes, (ii) ligating universal adapters to both ends and (iii) separating into single strands, they are ready for immobilization and amplification. The two most common methods are emulsion PCR (emPCR) and bridge PCR (bPCR). In emPCR the library molecules are attached to DNA-capture magnetic beads through an immobilized primer under conditions that favor one DNA molecule per bead. The beads are emulsified with the amplification reagents (polymerase, dNTP, second primer) in a water-in-oil mixture. Each bead is captured within a microreactor where temperature cycling PCR occurs. This results in the formation of bead-immobilized, clonally amplified DNA fragments, which are deposited into individual wells of picotiter plate and sequenced.

In bPCR, forward and reverse primers are attached to the solid surface of a flowcell and bridge PCR is performed isothermally using formamide as a denaturing agent instead of heat. Thus, each cycle of bPCR consists of flushing steps of denaturation, annealing, extension, and washing solutions. Thus, emPCR and bPCR are difficult reactions requiring thermo-cycling and solution-cycling, respectively. In addition, due to monomolecular nature of interactions at solid support, product self-annealing dominates over priming, which severely decreases PCR efficiency.

Recently an isothermal method, (known as “Wildfire”), was developed (5500×1 Wildfire, Life Technologies), which takes advantage of the monomolecular nature of priming process of immobilized primers. After hybridization to immobilized primers, the DNA fragment is replicated. Next, due to “breathing” (unfolding), the DNA fragment “walks” to another primer. After another cycle of replication, the first amplicon is displaced and primed at the free end by a second primer, which is free in solution. At this point, the process becomes exponential. The Wildfire clone generation simpler than emPCR or bPCR. However, it requires an initial temperature step for library hybridization to solid phase primers and relies on unspecific unfolding of DNA ends. In addition, strand-displacement priming from the free end of the DNA is undesired, since this can result in diffusion of amplicons, which could initiate new clone formation somewhere else. Thus, the Wildfire approach relies on two different priming processes. In the first, immobilized primers are able to displace previous (already extended) primers isothermally and initiate amplification. In the second priming events, the primers should not have this primer-displacement ability and should prime only after PBS is released.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

In an overarching aspect, the present invention provides an amplification system that reduces or eliminates the shortcomings of PCR, and the shortcomings of library preparation and clone generation in sequencing, as described above. For example, various aspects of the present invention provide methods and reaction components that (i) allow for completely isothermal amplification for detection of target nucleic acid; (ii) allow non-enzymatic amplification for detection of target nucleic acid; and (iii) can be used to identify amplicons without having to create separate individual probes for each target nucleic acid. Other aspects of the present invention provide methods and components that allow for the use of mono-adapters during library preparation, and allow for isothermal generation of nucleic acid clones that (i) eliminate the typical enrichment step of the process, (ii) use very little genomic material, and (iii) make a paired-end sequencing reaction a part of any sequencing.

Many aspects of the invention described herein relate to amplification using primers having dissociative sequences—DSPA. And so a brief description of that amplification process follows. However, while the DSPA process described below may, at times, refer to “quadruplexes” or other particular dissociative structures, it will be recognized by those of ordinary skill in the art that the invention is not limited to any particular sequence, or to a sequence that forms a quadruplex or any other particular structure, and that any sequence that dissociates from a DNA duplex to form another structure (whether quadruplex or a structure other than a quadruplex) may be used in accordance with the principles of the present invention. Further, as described above, other amplification processes may be compatible with DSPA. For example, helicase-dependent amplification (wherein a helicase is used to unwind the DNA duplex without having to alter the temperature of the reaction) may be compatible with DSPA (e.g., helicases may be used in the DSPA process).

As described above, PCR is limited by competition between primer binding and undesired self-annealing of target DNA. One aspect of DSPA inhibits self-annealing by providing at least one primer for amplification of a target nucleic acid (e.g., DNA), wherein the primer is adapted to conform into a dissociative structure—such as a quadruplex. The primer may conform into the dissociative structure during the extension step of PCR. As this occurs, the primer necessarily separates from its binding site on the target DNA sequence while the extending portion (DNA polymerase adding dNTPs to the sequence) remains bound (at least temporarily) in a double-stranded configuration.

Further, the primers used in DSPA may be universal. In other words, each primer in the primers used in the reaction includes the same sequence (or a substantially similar sequence that allows amplification to occur). As is known to those of ordinary skill in the art, in standard PCR at least two different primers (i.e., having two different sequences) are used (i.e., a first set of primers, wherein each primer of the first set includes the same or similar sequence, and a second set of primers wherein each primer of the second set includes the same or similar sequence, that sequence being different from the sequence of the primers in the first set). The need for the two sets of primers is to provide for amplification using each of the single strands from the DNA. Thus, one strand will be replicated using primers from the first set. The second strand (which is complementary to the first strand) will also be replicated. However, as that second strand is complementary to the first strand, the primers of the second set may be complementary to the primers of the first set. This creates the problem of primer-dimers (when the primers hybridize to one another—rather than to the target template denatured DNA strands). However, in DSPA, each of the primers used have the same or similar sequence. There is no second set of complementary primers that is needed. As such, there are no other complementary primers for the primers of the present application to hybridize to, thus eliminating the problem of primer dimers. Further, as the end being extended is currently bound with the target region, self-annealing of product is eliminated. And, the original primer binding site on the target DNA region is open for binding of another primer.

While “at least one primer” and a site being “open for binding of another primer” are discussed herein, it will be recognized by those of ordinary skill in the art that the “at least one primer” and the “another primer” may have the same sequence (or substantially similar sequence) as it is known that PCR employs multiple copies of a primer for amplification of a target DNA sequence. Further, as is known to those of ordinary skill in the art, the reaction components generally include multiple copies of primers. Thus, it will be understood that “at least one primer” or “one primer” or “a primer” or “the primer” or like references may refer to a single primer, or a primer among a set of multiple copies of the same or similar primers. Further, while various PCR procedures described herein are discussed as amplifying DNA, those of ordinary skill in the art will recognize that does not limit the disclosure to those seeking DNA sequences, as procedures such as reverse transcription PCR are well known, wherein reverse transcriptase reverse transcribes RNA into cDNA, which is then amplified by PCR.

Further, various nucleic acids, segments thereof, sequences, etc. are referred to herein as being “complementary” to one another. As used herein, “complementary” does not require an exact base-pair matching between each base of complementary sequences, for example. It only requires enough of a match that the sequences are capable of hybridizing.

Thus, the primer(s) in DSPA may be based on any sequence that is capable of forming a structure that allows or causes the primer to dissociate from a double-stranded DNA and form the particular structure—e.g., a quadruplex—such as during an extension step of PCR. One aspect of DSPA, then, uses the free energy of dissociative structures (such as quadruplexes) to drive unfavorable (endergonic) reactions of nucleic acids (e.g., isothermal PCR). One key point of these reactions is that some sequences—e.g., some G-rich sequences—are capable of forming structures/conformations with significantly more favorable thermodynamics than the corresponding DNA duplexes. The sequences are incorporated within DNA duplexes, which after interaction with an initiator (e.g., DNA polymerase) self-dissociate from the complementary strand and fold into their dissociative structures (e.g., quadruplexes). The energy of formation of these structures is used to drive PCR at substantially constant temperature.

Because DSPA inhibits product self-annealing and increases the number of PCR cycles within the exponential growth phase, the efficiency of PCR is improved by elongating the window of exponential amplification. And, since the dissociative structure is more stable than its corresponding duplex, unfolding of the duplex or release of target for the coming primers can occur without the need of substantial temperature change or any temperature change. In other words, in standard PCR, following the extension step, the DNA is in a duplex form. The next cycle then begins by raising the temperature to a point that the double-stranded DNA again denatures (i.e., separates into single strands). This is necessary in order to provide the separated sense and antisense DNA strands for primer binding (to each of the strands), followed by elongation during the next extension step (once the temperature of the reaction is reduced). However, by using primers based on the principles of DSPA, the primers and extending nucleotides that are added during the extension step naturally conform into the dissociative structure (such as a quadruplex). As this occurs, the primer (forming the dissociative structure) naturally separates from the target DNA sequence complementary to the primer, thereby leaving the target region complementary to the primer exposed in single-stranded form for binding of the next primer. This occurs without requiring raising of the temperature to denature the strands from one another. Thus, once begun, DSPA can proceed under isothermal conditions. As described above, isothermal DNA amplification is desirable because it would not require expensive instrumentation for thermocycling (as does standard PCR) and thus would allow for DNA amplification in the field and at point-of-care.

However, as described in the Background, DSPA may not be completely isothermal due to the need to incorporate primer binding sites (PBS) into the target DNA, by using cycles of traditional PCR. One aspect of the present invention, then, describes an amplification process or processes that is/are completely isothermal such that it/they would be suitable for point-of-care analysis.

In general, this aspect of the present invention includes at least one nucleic acid construct including first, second and third sequence segments. This construct may be used to identify target nucleic acid via an amplification process (in this process, it may be the construct itself that is amplified—with the amplification of the construct signifying the presence of target nucleic acid). At least a portion of the first sequence segment includes a sequence adapted to conform into a structure that dissociates from a complementary strand of a DNA duplex (i.e., a dissociative sequence adapted to form a dissociative structure). At least a portion of the second sequence segment includes a sequence that is complementary to a target nucleic acid. And, at least a portion of the third sequence segment includes a sequence that is complementary to the dissociative sequence portion of the first sequence segment. The nucleic acid construct may include a detectable label, so that the presence of the target nucleic acid can be confirmed, for example.

The nucleic acid construct may be in the form of a stem-loop. The stem region is formed by the dissociative-structure-forming sequence (i.e., first sequence segment) duplexed with its complementary strand (i.e., third sequence segment). The loop region includes the second sequence segment (which is complementary to a target nucleic acid sequence). Target nucleic acid binds to the loop region of the construct and unfolds it, which releases the third sequence segment from the first sequence segment. This frees the third sequence segment to be bound by primers (having a sequence complementary to the third sequence segment), thereby initiating the amplification reaction. In other words, the third sequence segment provides a PBS. Further, the dissociative-structure-forming sequence included in the first sequence segment of the nucleic acid is a sequence such as would be used for a primer in DSPA.

In order to perform the amplification reaction, then, the stem-loop nucleic acid construct described above is combined with target nucleic acid (or with a sample that one wants to test for the presence of a particular target nucleic acid) and at least one primer having a dissociative-structure-forming sequence as described in the first sequence segment (although these primers are free in solution and are not part of the stem-loop construct described above). Thus, when the target nucleic acid hybridizes with the loop region of the stem-loop construct, the stem-loop is unfolded. This unfolding releases the third sequence segment (having a PBS) from the first sequence segment. The first sequence segment will then form its dissociative structure (such as a quadruplex), and the PBS of the third sequence segment remains free for the primers in the mixture to bind thereto and start an amplification reaction. Once a primer attaches to the PBS, it replicates the unfolded loop portion (i.e., the probe) of the stem-loop construct during extension. As this occurs, the target nucleic acid will be released from the duplex of unfolded stem-loop and primer-extending-sequence, thereby allowing the target nucleic acid to be free for binding to another stem-loop construct. This approach has at least two advantages: (i) it allows design of a truly isothermal mechanism; and (ii) it can be used in detection of RNA pathogens, such as HIV, without reverse transcription.

Further, as described in the Background, PCR, which depends on the enzymatic activity of DNA polymerases, is not ideally suited for point-of-care use. Thus, another aspect of the present invention provides protein-free dissociative-structure-based amplification. In general, this aspect of the present invention provides a mixture of nucleic acid constructs including at least one first nucleic acid construct and at least one second nucleic acid construct. The at least one first nucleic acid construct and at least one second nucleic acid construct are designed such that they work in concert to provide an amplification reaction that can identify a target nucleic acid sequence (e.g., a DNA sequence) without the use of enzymes as in standard PCR. The reaction in this aspect of the present invention may also proceed isothermally.

To that end, the first nucleic acid construct includes a first sequence segment and a second sequence segment, wherein at least a portion of the first sequence segment includes a sequence adapted to conform into a structure that dissociates from a complementary strand of a DNA duplex, and wherein at least a portion of the second sequence segment includes a sequence that is complementary to a target nucleic acid. And, the second nucleic acid construct includes a first sequence segment and a second sequence segment, wherein at least a portion of the first sequence segment includes a sequence adapted to conform into a structure that dissociates from a complementary strand of a DNA duplex, and wherein at least a portion of the second sequence segment includes a sequence that is substantially similar to the target nucleic acid such that the second sequence segment of the second nucleic acid construct can bind with the second sequence segment of the first nucleic acid construct.

Like the nucleic acid construct for isothermal amplification and identification described above, each of the first and second nucleic acid constructs in this aspect of the present invention may be provided in the form of a stem-loop nucleic acid construct. In each of the stem-loop constructs, the portion of the sequence which includes a dissociative-structure-forming sequence provides a portion of the stem (being duplexed with a complementary sequence). A primary portion of the loop of the first nucleic acid construct includes a sequence that is complementary to the target nucleic acid, and a primary portion of the loop of the second nucleic acid construct includes a sequence that is substantially the same as the target nucleic acid sequence.

When the first and second nucleic acid constructs of this aspect of the present invention are combined with target nucleic acid, the target nucleic acid hybridizes with the loop segment of the first nucleic acid construct. This unfolds the stem-loop of the first nucleic acid construct, thereby unwinding the stem. Once unwound, the sequence of the now free dissociative-structure-forming sequence (i.e., the first sequence segment) forms its dissociative structure (e.g., a quadruplex). As a result, the DNA duplex between the loop portion and the target nucleic acid is destabilized and the complex quickly dissociates. The released target binds to another first nucleic acid stem-loop construct and repeats the same cycle.

Meanwhile, the denatured first nucleic acid construct, now having a dissociative structure at its 5′ end, binds to the stem-loop of the second nucleic acid construct [with hybridization between the second sequence segment of the first nucleic acid construct and the loop segment (second sequence segment) of the second nucleic acid construct]. This induces a similar unwinding/dissociation process in the second nucleic acid construct. Once unwound, the first sequence segment of the second nucleic acid construct forms its dissociative structure (e.g., a quadruplex). As a result, the DNA duplex between the first and second nucleic acid constructs is destabilized, and the two separate. The released second nucleic acid construct now binds to and unfolds stem-loop of another first nucleic acid by denatured second nucleic acid. At this point, the reaction becomes autocatalytic, i.e., the product of each cycle serves as the catalyst for the subsequent cycles. Thus, amplification occurs in the absence of any standard DNA polymerases, and can proceed isothermally.

Further, as described above, a drawback of current quantification systems is that they use FRET-based applications (Förster Resonance Energy Transfer), which require costly synthesis and considerable effort to design a sensitive probe. As is known to those of ordinary skill in the art, FRET is a mechanism describing energy transfer between two chromophores. A donor chromophore, initially in its electronic excited state, may transfer energy to an acceptor chromophore (in proximity, typically less than 10 nm) through nonradiative dipole—dipole coupling. Further, the processes currently used require multiple probes for multiple targets (i.e., one probe for each target), which greatly increases materials, time, and expense.

Thus, another aspect of the present invention provides FRET-based detection that increases the multiplex capability of DSPA. A fluorescent nucleotide donor is placed internally and a fluorescent acceptor is attached at 5′-end of a DSPA primer. The fluorescence emission peak of the donor overlaps the excitation peak of attached acceptor.

In another aspect of the invention, a nucleic acid construct may be provided that including multiple sequence segments, each having a detectable label, that allows for amplification of the signal generated.

More specifically, the nucleic acid construct may include (1) a first sequence strand, and (2) plurality of nucleotide segments, wherein the plurality of nucleotide segments each include a sequence that is complementary to at least a portion of the sequence of the first sequence strand. As a result, the plurality of nucleotide segments can act as a segmented version of a complementary strand, and, at least initially, retain the first sequence strand in a pseudo-duplex form (e.g., a duplex including one complete strand having bound thereto multiple fragments of a “second strand”).

The first sequence strand of nucleotides includes from the 5′ to the 3′ end: (1) a first segment having a sequence of nucleotides complementary to a target nucleic acid, and (2) a plurality of segments, each of the plurality of segments having a detectable label. Each of the plurality of segments is adapted to conform into a conformation having a free energy with more favorable thermodynamics than a corresponding B-DNA duplex. For example, each of the plurality of segments may be adapted to conform into a quadruplex.

As described above, the plurality of segments initially retains the first sequence strand in a pseudo-duplex form. This is accomplished because each of the plurality of nucleotide segments includes a sequence that is complementary to either (1) a sequence spanning the first segment and one of the plurality of segments of the first sequence strand, or (2) at least two of the plurality of segments of the first sequence strand.

When target nucleic acid hybridizes with its complementary part of the first sequence strand, the first of the plurality of segments is displaced. This is followed by first quadruplex (or other non-B-DNA duplex) formation, which in turn destabilizes next bimolecular duplex and so on. As the quadruplexes (or other non-B-DNA conformations) form, the labels are detectable, and the multiple labels provide an amplified signal.

### BRIEF DESCRIPTION OF THE DRAWINGS

The accompanying drawings, which are incorporated in and constitute a part of this specification, illustrate embodiments of the invention and, together with the general description of the invention given above and the detailed description of the embodiments given below, serve to explain the principles of the present invention.

FIG. 1 is a graph showing a typical RT-PCR curve.

FIG. 2 is a schematic illustration of the DSPA process.

FIG. 3 shows the incorporation of the DSPA target site (dotted portion) in templates by attachment of quadruplex forming sequences (hash-marked portion) to primers.

FIG. 4A is a schematic of isothermal signal amplification showing an exponential growth pattern.

FIG. 4B is a schematic of isothermal signal amplification showing a linear growth pattern.

FIG. 5 is a schematic of a DNA G-quartet.

FIG. 6 is a schematic of structures and modes of action of previous probes with panel A showing a molecular beacon, panel B showing a TaqMan®, and panel C showing a Scorpion™ probe.

FIG. 7 is a schematic of non-enzymatic signal amplification.

FIG. 8 is a schematic showing an example of FRET between 2Ap and Alexa405 upon quadruplex formation.

FIG. 9 is a graph showing fluorescence melting curves of single-stranded oligonucleotides.

FIG. 10 shows fluorescence melting curves of G3T-ds15 duplex (i.e., GGG(2Ap)GGGTGGGTGGG [SEQ. ID. NO. 1] (“2Ap-G3T”) in duplex form with its complementary strand CCCACCCACCCTCCC [SEQ. ID. NO. 2]), wherein the black and squared lines correspond to heating and cooling (at 1° C./min rate), respectively.

FIG. 11 shows fluorescence melting curves of a 2Ap-G3T duplex in 15 mM KCl, 35 mM CsCl, 2 mM MgCl₂, 20 mM Tris-HCl, pH 8.7 wherein the black and squared lines correspond to heating and cooling (at 1° C./min rate), respectively.

FIG. 12 shows UV melting curves of G3T-ds15 and G3T-ds13 (i.e., GGG(2Ap)GGGTGGGTG [SEQ. ID. NO. 3] in duplex form with a complementary strand CCCTCCCACCCACCC [SEQ. ID. NO. 4]) duplexes in the presence (-∘- and -□-) and absence (-Δ- and black line) of K⁺ ions.

FIG. 13 includes schematic diagrams showing two possible structures of (GGGT)₄ [SEQ. ID. NO. 5] with panel A showing an antiparallel conformation based on NMR work, and with panel B showing a parallel conformation suggested on the bases of thermodynamic and spectral studies.

FIG. 14 is fluorescence spectra of GGG(6MI)GGGCGGGCGGG [SEQ. ID. NO. 6] without and with its complementary strand.

FIG. 15 is a schematic of a nucleic acid construct including multiple sequences having fluorescent nucleotides for multiplexing of signal.

FIG. 16 shows (1) in panel A, a sequence of an exemplary stem loop probe (the 5′ to 3′ sequence at the bottom of panel A, with primer binder site underlined with hash-marked line, loop portion underlined, and dissociative-structure-forming portion underlined by dotted segment) as would be used in linear amplification (as shown in FIG. 4B) unfolded and bound to target nucleic acid (underlined by dashed line in panel A) and primer (underlined by dotted segment of upper sequence in panel A); and (2) in panels B and C, graphs demonstrating that the stem loop probe of this embodiment leaks since its 3′ end is able to form a dissociative structure, such as a quadruplex.

FIG. 17 shows (1) in panel A, a sequence of an exemplary stem loop probe (the 5′ to 3′ sequence at the bottom of panel A, with primer binder site underlined with hash-marked line, loop portion underlined, and dissociative-structure-forming portion underlined by dotted segment) including one CC mismatch, which prevents the 3′ end from forming a dissociative structure (such as a quadruplex) and a primer GG mismatch at the 5′ end; and (2) in panels B and C, graphs which demonstrate that leakage (such as shown in FIG. 16) is reduced and eliminated by inhibiting the formation of the dissociative structure.

FIG. 18 is a schematic of isothermal signal amplification showing a linear growth pattern, and using an example of a stem loop probe such as that as shown in FIG. 17.

FIGS. 19A-C are schematics showing reaction mixtures, and demonstrating how DSPA simplifies the reaction mixture (with FIG. 19A showing the reaction mixture for typical PCR/immuno-PCR, FIG. 19B showing the reaction mixture for SLP (stem-loop probe)-DSPA, and FIG. 19C showing the reaction mixture for immuno-DSPA).

FIGS. 20A-D are schematics showing modularity of recognition and signal production using DSPA, and additionally showing the use of an applied magnetic field to progress nucleic acid adsorbed onto metal particles through solutions.

FIGS. 21A and B are schematics showing the universal primer/probe nature of DSPA in multi-well diagnostics, and the use of an applied magnetic field to progress nucleic acid adsorbed onto metal particles through solutions.

FIGS. 22A and B are schematics showing the monomolecular nature of detection using DSPA.

FIG. 23 is another schematic showing the use of DSPA in multi-well diagnostics, and the use of an applied magnetic field to progress nucleic acid adsorbed onto metal particles through solutions.

FIG. 24 is a schematic showing exponential DSPA using two primers (a first primer being a DSPA primer, and a second primer being a non-DSPA primer) for nucleic acid(s) attached to a support (e.g., magnetic beads), wherein the probe contains three separate segments: pathogen complement, second primer, and DSPA PBS.

FIG. 25 is a schematic of exponential DSPA using a single DSPA primer.

FIG. 26 is a schematic showing linear DSPA or immuno-DSPA in multi-well diagnostics, and the use of an applied magnetic field to progress nucleic acid adsorbed onto metal particles through solutions.

FIG. 27A is a schematic showing the principles of nicking DSPA.

FIG. 27B includes a graph showing experimental data comparing linear nicking DSPA to linear DSPA.

FIG. 27C is another schematic showing the principles of nicking DSPA.

FIG. 28 includes experimental data showing exponential DSPA at differing probe concentrations, wherein panel A shows representative amplifications of the probe molecules prepared by 10-fold dilutions ranging between 100 μM and 100 aM concentration, and panel B shows the correlation between threshold time, collected at 7500 RFU, and the logarithm of the probe concentration. In FIG. 28, experimental conditions: 350 nM DSPA primers, 300 nM left primer, 400 μM dNTP, 0.06 U/μl Vent (exo-). Buffer:10 mM KCl, 40 mM CsCl, 2 mM MgCl₂ at 66° C. And in FIG. 28, panel A, the following concentrations are shown: 100 μM, 10 μM, 1 μM, 100 fM, 10 fM, 1 fM, 100 aM, and negative control (no probe).

FIG. 29 is a graph showing an example of exponential DSPA using a truncated left primer at different template concentrations. In FIG. 29, the experimental conditions are as follows: 350 nM DSPA primers, 300 nM left primer, 400 μM dNTP, 0.08 U/μl Vent (exo-). Buffer:5 mM KCl, 45 mM CsCl, 2 mM MgCl₂ at 66° C.

FIG. 30 is a graph showing an example of exponential DSPA using stabilized primers at different template concentration. In FIG. 30, the experimental conditions are as follows: 300 nM DSPA primers, 400 nM left primer, 400 μM dNTP, 0.06 U/μl Vent (exo-). Buffer:15 mM KCl, 35 mM CsCl, 2 mM MgCl₂ at 69° C.

FIG. 31 is a schematic showing the process for exponential and isothermal DSPA that can be performed in a single housing (as opposed to a multi-well housing).

FIG. 32 is a schematic showing the process for exponential DSPA using four primers and performed in a single housing (as opposed to a multi-well housing).

FIG. 33 is a schematic showing the process for exponential DSPA using four primers and performed in a single housing.

FIG. 34 is a schematic showing DSPA using two primers (a first primer being a DSPA primer, and a second primer being a non-DSPA primer) with the second primer attached to a solid support.

FIG. 35 is a schematic showing DSPA using two primers (a first primer being a DSPA primer, and a second primer being a non-DSPA primer) with the first primer (i.e., the DSPA primer) attached to a solid support.

FIG. 36A is a schematic showing parallel amplification via DSPA using a single primer, with the primer attached to a solid support.

FIG. 36B is a schematic showing bridge amplification via DSPA using a single primer, with the primer attached to a solid support.

FIGS. 37A-D are schematics showing clone generation for mono-adapter DSPA.

FIGS. 38A-38C are schematics showing an outline of linear DSPA, which uses free energy of G3T quadruplex to drive isothermal amplification of DNA signal and detects amplicons through fluorescence of 3MI, incorporated in the primers. In the figure, panel A is a schematic diagram of a G3T quadruplex with all parallel G-tracts and chain-reversal T-loops.

FIGS. 39A and 39B show a real-time assay for monitoring quadruplex unfolding by DNA polymerases. FIG. 39A shows an assay scheme and construct sequences. In this assay, a quadruplex with 2AP (shown as a blue segment or A) is attached to a 20-nt long PBS. Primer extension can only occur upon quadruplex unfolding, which is accompanied by fluorescence quenching and can be detected in real-time. FIG. 39B shows representative unfolding curves at different temperatures in 5 mM KCl, 45 mM CsCl, 2 mM MgCl₂ using Taq (thinner lines correspond to control reactions in the absence of the polymerase).

FIG. 40 shows exponential DSPA conducted by different DNA polymerases. The upper panel shows template and primer sequences. M represents 3MI in the DSPA primer. Solid curves correspond to the amplification in the presence of 100 μM template and dashed lines corresponds to NTC. Experimental conditions are as follows: 350 nM DSPA primer, 300 nM left prime, 400 μM dNTP, 0.06 U/μl Vent (exo-), 0.08 U/μl Bst 2.0, 0.1 U/μl Taq. Buffer:10 mM KCl, 40 mM CsCl, 2 mM MgCl₂ at 66° C.

FIGS. 41A-41C show no template controls (NTC) with different primers. FIG. 41A shows primer sequences; M denotes 3MI in the DSPA primers. FIG. 41B shows NTC using a 13-nt DSPA primer. And FIG. 41C shows 14-nt DSPA primer. Experimental conditions are as follows: 300 nM DSPA primers, 300 nM left primers, 400 μM dNTP, 0.06 U/μl Vent (exo-). Buffer: 10 mM KCl, 40 mM CsCl, 2 mM MgCl₂ at 66° C.

FIG. 42 shows DSPA rate vs. temperature profiles. DSPA rates were determined from the initial slopes of the kinetic curves conducted at 1 μM primer, 1 nM target, 800 μM dNTP, 0.05 U/μl Taq in the presence of 25 mM K+.

FIG. 43 shows fluorescence melting of the quadruplex-containing template shown in FIG. 39A. The melting experiments are performed in the presence of different K+ concentration, which demonstrates strong stabilization effect of the cation. Each solution contained 50 mM monovalent cations (K++Cs+), 2 mM MgCl₂, 10 mM Tris-HCl at pH 8.7.

FIGS. 44A and 44B show quadruplex unfolding by DNA polymerases studied by the assay described in FIG. 39A. FIG. 44A: in the presence of 25 mM K+ at 68° C. using Bst 2.0 and Taq; FIG. 44B: in the presence of 5 mM K+ at 65° C. using Bst 2.0, Taq, Vent and Vent(exo-). Common experimental conditions: 1 μM template with quadruplex, 1 μM primer, 800 μM dNTP; 0.05 U/μl DNA polymerases.

FIGS. 45A and 45B show optimal K+ concentration of exponential DSPA studied on the system shown in FIG. 40. FIG. 44A: The curves characterize amplification system with separate processes. First, increase in fluorescence, corresponds to linear DSPA (350 nM QPA primer, 10 nM template, 800 μM dNTP; 0.04 U/μl Vent (exo-) at 66° C.), which demonstrates robust activity and plateaus around 30 min. The second process starts by adding 1 μM left primer (at 37 min), which binds to extended strands and initiates quadruplex unfolding accompanied by fluorescence quenching. As expected, unfolding is faster at lower concentrations of K+ due to decreased stability of the quadruplexes. FIG. 44B: Exponential DSPA with 100 μM template (see FIG. 39) at different K+ concentration (500 nM DSPA primer, 100 nM left primer). The experiment demonstrates faster amplification in the presence of 10 mM K+.

FIGS. 46A and 46B show an optimal primer ratio of exponential DSPA. These experiments were conducted for the system described in FIG. 40 at 100 μM template, 800 μM dNTP, 0.04 U/μl Vent (exo-), 10 mM K+ at 66° C. in the presence of 500 nM (A) and 350 nM (B) DSPA primers. Varying concentrations of the left primers are shown on the figures.

### DETAILED DESCRIPTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

In an overarching aspect, the present invention provides an amplification system that reduces or eliminates the shortcomings of PCR, and the shortcomings of library preparation and clone generation in sequencing, as described above. For example, various aspects of the present invention provide methods and reaction components that (i) allow for completely isothermal amplification for detection of target nucleic acid; (ii) allow non-enzymatic amplification for detection of target nucleic acid; and (iii) can be used to identify amplicons without having to create separate individual probes for each target nucleic acid. Other aspects of the present invention provide methods and components that allow for the use of mono-adapters during library preparation, and allow for isothermal generation of nucleic acid clones that (i) eliminate the typical enrichment step of the process, (ii) use very little genomic material, and (iii) make a paired-end sequencing reaction a part of any sequencing.

Many aspects of the invention described herein relate to amplification using primers having dissociative sequences—DSPA—as well as other reaction components having dissociative sequences. And so a brief description of that amplification process follows. However, while the DSPA process described below may, at times, refer to “quadruplexes” or other particular dissociative structures, it will be recognized by those of ordinary skill in the art that the invention is not limited to any particular sequence, or to a sequence that forms a quadruplex or any other particular structure, and that any sequence that dissociates from a DNA duplex to form another structure (whether quadruplex or a structure other than a quadruplex) may be used in accordance with the principles of the present invention. Further, as described above, other amplification processes may be compatible with DSPA. For example, helicase-dependent amplification (wherein a helicase is used to unwind the DNA duplex without having to alter the temperature of the reaction) may be compatible with DSPA (e.g., helicases may be used in the DSPA process).

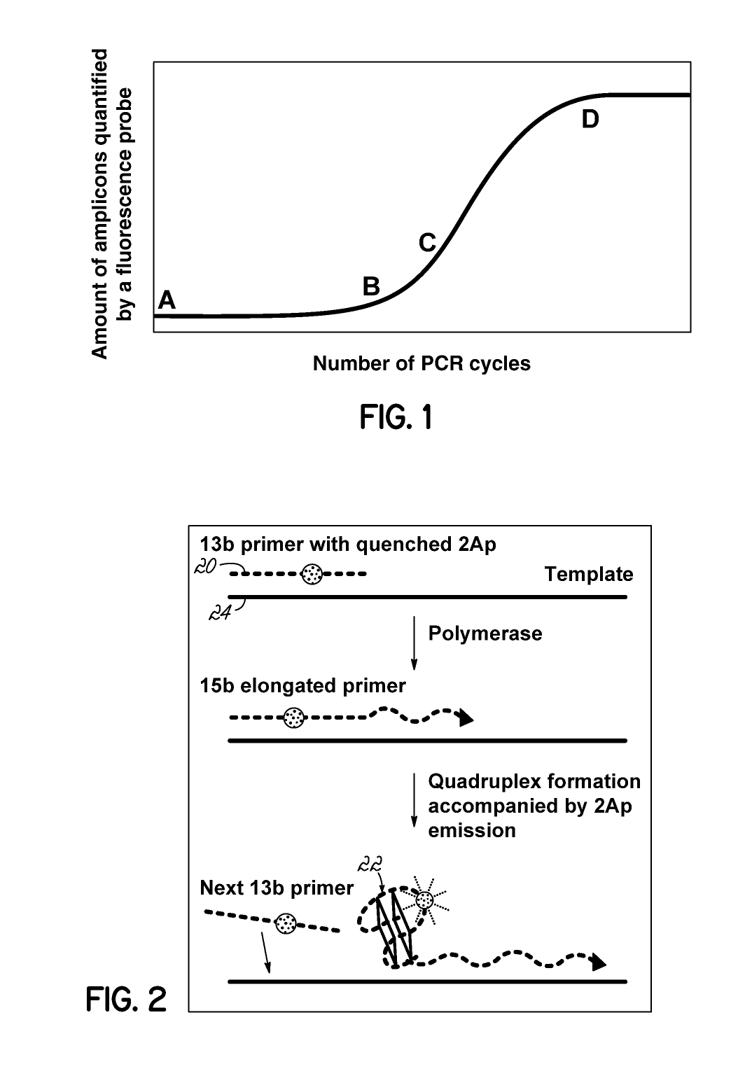

As described above, PCR is limited by competition between primer binding and undesired self-annealing of target DNA. Referring to FIG. 1, which shows a typical PCR, at the initial stage of PCR, product molecules are at low enough concentrations that product self-annealing does not compete with primer binding and amplification proceeds at an exponential rate (see the AC segment, FIG. 1; the AB segment corresponds to exponential phase undetectable by fluorescence measurements). However, with accumulation of product DNA, self-annealing becomes dominant and PCR slows (CD segment, FIG. 1) and eventually DNA amplification ceases (plateau, FIG. 1).

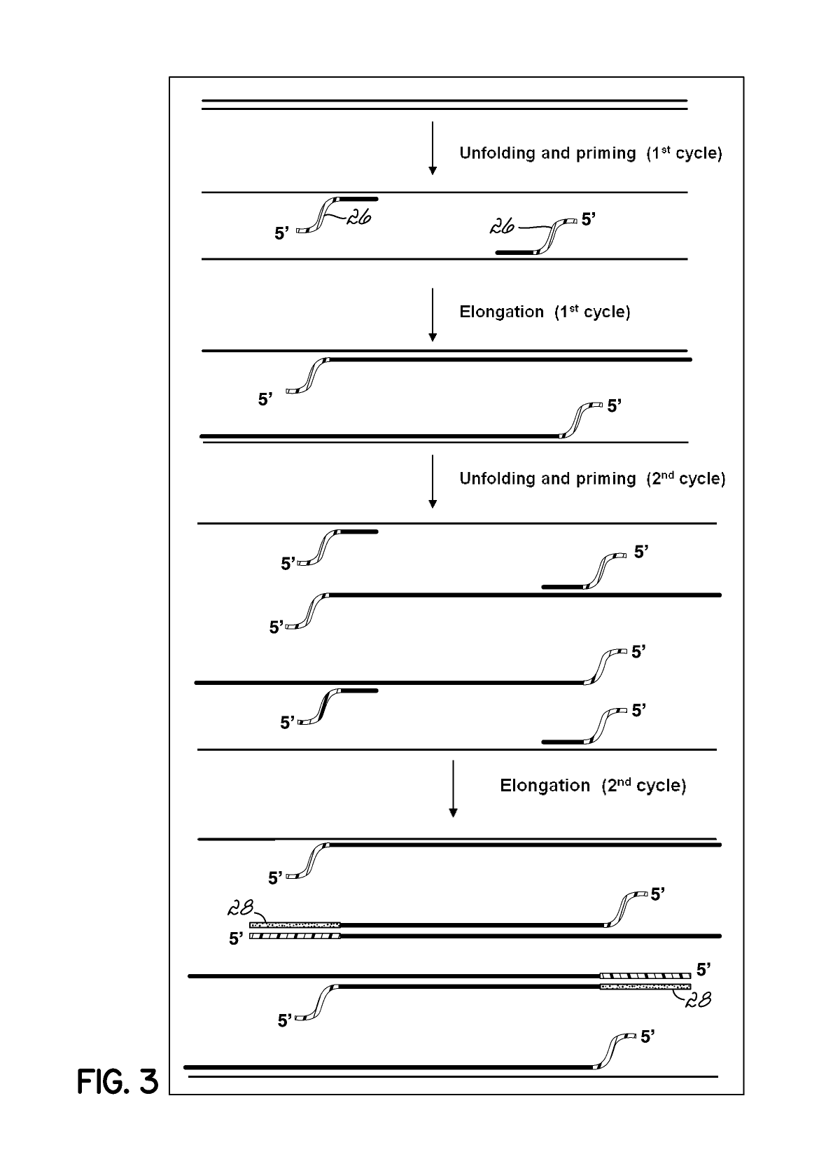

One aspect of DSPA inhibits self-annealing by providing at least one primer, such as an oligonucleotide primer, for amplification of a target nucleic acid (e.g., DNA), wherein the primer is adapted to conform into a structure that can dissociate from a DNA duplex structure in the absence of heating. In other words, and referring to FIG. 2, the primer 20 includes a sequence that naturally conforms into a structure, such as a quadruplex structure (or any other non-B DNA configuration or other DNA structure) in which intramolecular base pairing allows or causes the primer to dissociate from the double stranded DNA—of which it is one strand—and form its particular structure. This structure may be referred to herein as a “dissociative structure” 22 or “dissociative conformation” or the like. As one nonlimiting example, the primer may be adapted to conform into a quadruplex structure. The primer may conform into the quadruplex structure during an extension step of PCR. As this occurs, the primer necessarily separates from its binding site on the target DNA sequence 24 while the extending portion (DNA polymerase adding dNTPs to the sequence) remains bound (at least temporarily) in a double-stranded configuration. Again, it will be recognized by those of skill in the art that quadruplex structures are merely exemplary, and the primer may form any other structure that can disassociate from any DNA configuration of which it is a part.

Further, the primers used in DSPA may be universal. In other words, each primer in the primers used in the reaction includes the same sequence (or a substantially similar sequence that allows amplification to occur). As is known to those of ordinary skill in the art, in standard PCR at least two different primers (i.e., having two different sequences) are used (i.e., a first set of primers, wherein each primer of the first set includes the same or similar sequence, and a second set of primers wherein each primer of the second set includes the same or similar sequence, that sequence being different from the sequence of the primers in the first set). The need for the two sets of primers is to provide for amplification using each of the single strands from the DNA. Thus, one strand will be replicated using primers from the first set. The second strand (which is complementary to the first strand) will also be replicated. However, as that second strand is complementary to the first strand, the primers of the second set may be complementary to the primers of the first set. This creates the problem of primer-dimers (when the primers hybridize to one another—rather than to the target template denatured DNA strands). However, in DSPA, each of the primers used have the same or similar sequence. There is no second set of complementary primers that is needed. As such, there are no other complementary primers for the primers of the present application to hybridize to, thus eliminating the problem of primer dimers. Further, as the end being extended is currently bound with the target region, self-annealing of product is eliminated. And, the original primer binding site on the target DNA region is open for binding of another primer.

While “at least one primer” and a site being “open for binding of another primer” are discussed herein, it will be recognized by those of ordinary skill in the art that the “at least one primer” and the “another primer” may have the same sequence (or substantially similar sequence) as it is known that PCR employs multiple copies of a primer for amplification of a target DNA sequence. Further, as is known to those of ordinary skill in the art, the reaction components generally include multiple copies of primers. Thus, it will be understood that “at least one primer” or “one primer” or “a primer” or “the primer” or like references may refer to a single primer, or a primer among a set of multiple copies of the same or similar primers. Further, while various PCR procedures described herein are discussed as amplifying DNA, those of ordinary skill in the art will recognize that does not limit the disclosure to those seeking DNA sequences, as procedures such as reverse transcription PCR are well known, wherein reverse transcriptase reverse transcribes RNA into cDNA, which is then amplified by PCR.

Further, various nucleic acids, segments thereof, sequences, etc. are referred to herein as being “complementary” to one another. As used herein, “complementary” does not require an exact base-pair matching between each base of complementary sequences, for example. It only requires enough of a match that the sequences are capable of hybridizing to one another.

One aspect of DSPA, then, uses the free energy of dissociative structures (such as quadruplexes) to drive unfavorable (endergonic) reactions of nucleic acids (e.g., isothermal PCR). A key point of these reactions is that some sequences—e.g., some G-rich sequences—are capable of forming structures/conformations with significantly more favorable thermodynamics than the corresponding DNA duplexes. The sequences are incorporated within DNA duplexes, which after interaction with an initiator (e.g., DNA polymerase) self-dissociate from the complementary strand and fold into their dissociative structures (e.g., quadruplexes). The energy of formation of these structures is used to drive PCR at substantially constant temperature.

Because DSPA inhibits product self-annealing and increases the number of PCR cycles within the exponential growth phase, the efficiency of PCR is improved by elongating the window of exponential amplification. And, since the dissociative structure is more stable than its corresponding duplex, unfolding of the duplex or release of target for the coming primers can occur without the need of substantial temperature change or any temperature change. In other words, in standard PCR, following the extension step, the DNA is in a duplex form. The next cycle then begins by raising the temperature to a point that the double-stranded DNA again denatures (i.e., separates into single strands). This is necessary in order to provide the separated sense and antisense DNA strands for primer binding (to each of the strands), followed by elongation during the next extension step (once the temperature of the reaction is reduced). However, by using primers based on the principles of DSPA, the primers and extending nucleotides that are added during the extension step naturally conform into the dissociative structure (such as a quadruplex). As this occurs, the primer (forming the dissociative structure) naturally separates from the target DNA sequence complementary to the primer, thereby leaving the target region complementary to the primer exposed in single-stranded form for binding of the next primer. This occurs without requiring raising of the temperature to denature the strands from one another. Thus, once begun, DSPA can proceed under isothermal conditions. As described above, isothermal DNA amplification is desirable because it would not require expensive instrumentation for thermocycling (as does standard PCR) and thus would allow for DNA amplification in the field and at point-of-care.

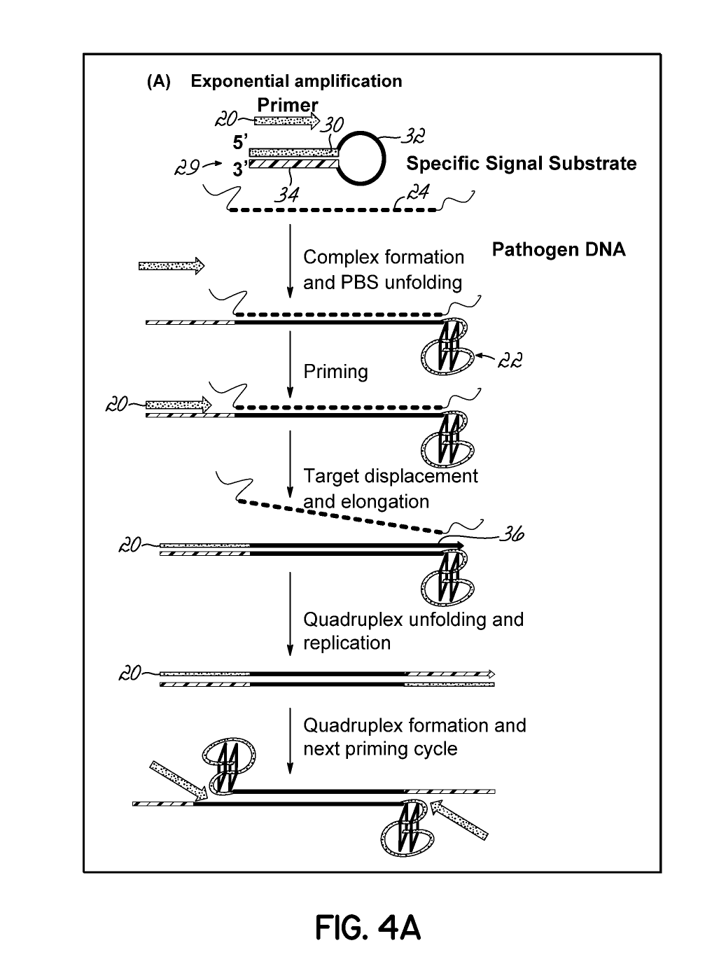

However, as described in the Background, DSPA is not completely isothermal due to the need to incorporate primer binding sites (PBS) into the target DNA, by using cycles of traditional PCR. The incorporation of the target sequence into a template is shown schematically in FIG. 3. The quadruplex folding sequence (hash-marked segment) 26 will be attached at the 5′-end of both forward and reverse primers. The products of the 2nd cycle (four duplexes at the end of PCR, FIG. 3) contain two single-stranded amplicons fully complementary to each other with incorporated target sites at the 3′-end (dotted segments) 28. Thus, at the end of the second cycle, the number of amplicons with incorporated target sites equals the initial amount of template. The use of the initial two cycles of traditional PCR to incorporate PBS prevents this version of DSPA from being completely isothermal. One aspect of the present invention, then, provides a nucleic amplification process that is completely isothermal such that it would be suitable for point-of-care analysis.

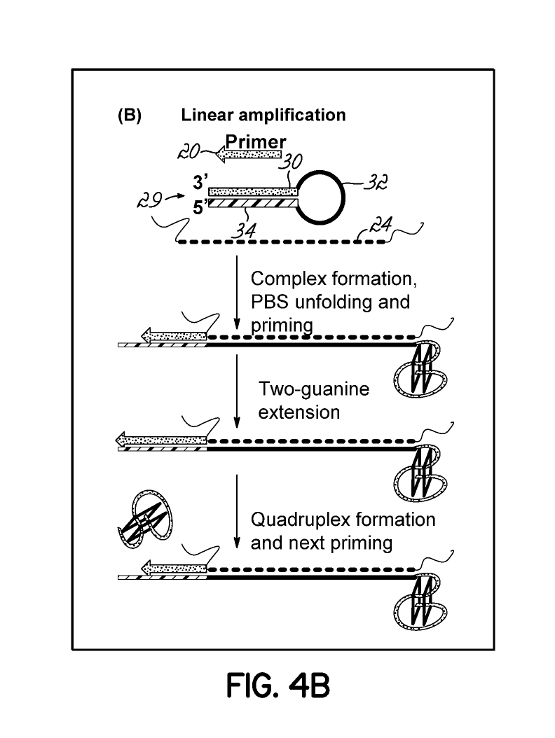

In general, and referring now to FIGS. 4A and 4B, this aspect of the present invention includes at least one nucleic acid construct 29 including first, second and third sequence segments 30, 32, 34 (dotted segment, black line, and hash-marked segments, respectively, in FIGS. 4A and 4B). This construct may be used to identify target nucleic acid 24 (dashed line) via an amplification process wherein it may be the construct itself that is amplified. At least a portion of the first sequence segment 30 includes a sequence adapted to conform into a structure that dissociates from a complementary strand of a DNA duplex (i.e., a dissociative sequence adapted to form a dissociative structure). At least a portion of the second sequence segment 32 includes a sequence that is complementary to a target nucleic acid. And, at least a portion of the third sequence segment 34 includes a sequence that is complementary to the dissociative sequence portion of the first sequence segment. The nucleic acid construct may include a detectable label, so that the presence of the target nucleic acid can be confirmed.

The nucleic acid construct of this aspect of the present invention may have a stem-loop configuration. As is known to those of ordinary skill in the art, stem-loop intramolecular base pairing is a pattern that can occur in single-stranded DNA, or in RNA. The structure may also be referred to as a “hairpin” or “hairpin loop.” It occurs when two regions of the same strand that are generally complementary in nucleotide sequence when read in opposite directions, base-pair to form a double helix that ends in an unpaired loop. In the nucleic acid construct of this aspect of the present invention, at least a portion of the loop region may be complementary to the nucleic acid of interest (i.e., the second sequence segment may be in the loop region). The stem region is then formed by the dissociative-structure-forming sequence (i.e., first sequence segment—30) duplexed with its complementary strand (i.e., third sequence segment—34). In the presence of target nucleic acid 24, the target nucleic acid binds to the loop region 32 of the nucleic acid construct and unfolds the construct, which releases the third sequence segment from the first sequence segment. This frees the third sequence segment (dashed/dotted line) to be bound by primers 20 (short arrow) having a sequence complementary to the third sequence segment, thereby initiating the amplification reaction. In other words, the third sequence segment provides a PBS. Further, the dissociative-structure-forming sequence included in the first sequence segment of the nucleic acid is a sequence such as would be used for a primer in DSPA. Due to the use of sequences that form dissociative structures, the primers and first sequence segments can dissociate from complementary sequences without having to change the temperature of the reaction.

This aspect of the present invention, then, provides an amplification process that is isothermal, which is accomplished, in one aspect, by changing the target that is amplified. For example, current real-time nucleic acid detection mechanisms usually are based on amplification of target nucleic acid followed by quantification. However, an aspect of the present invention provides a detection mechanism that amplifies a nucleic acid construct specific to the target of interest (rather than the target nucleic acid itself) for further quantification. This approach has at least two advantages: (i) it provides a truly isothermal mechanism; and (ii) it can be used in detection of RNA pathogens, such as HIV, without reverse transcription.

As described above, in one aspect, a portion of the first sequence segment may be based on any sequence that is capable of forming a structure that allows or causes that portion to dissociate from a duplex form, such as under isothermic conditions. And, as described above, an example of a structure which allows such dissociation is a quadruplex structure. As previously discussed, such quadruplex structures may be commonly formed by sequences rich in guanine residues. Thus, some of the discussion below is directed to G-rich sequences, which are capable of forming such quadruplex structures. However, it will be appreciated by those of ordinary skill in the art that the general and particular sequences described below, and the particular structures, such as quadruplex structures, described below, are merely exemplary and that there may be other useful sequences that form structures which allow dissociation from a double-stranded DNA form in accordance with the principles of the present invention.

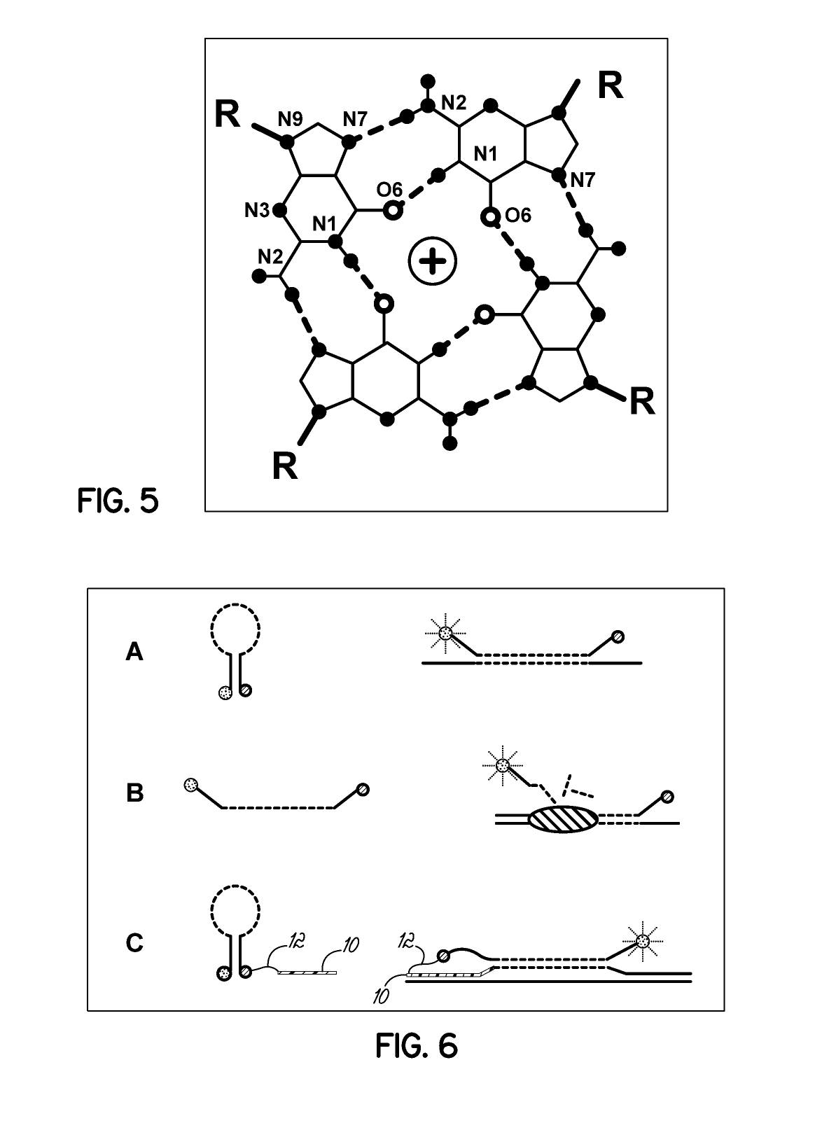

As is known to those of ordinary skill in the art, quadruplexes are high-ordered nucleic acid structures (DNA or RNA) formed from G-rich sequences that are built around tetrads of hydrogen bonded guanine bases (see FIG. 5). Thus, in order to provide a first sequence segment that can form quadruplexes, the first sequence segment of this aspect of the present invention may be designed with a sequence having a G content of a high enough amount (or to obtain a high enough amount) to allow the first sequence segment to conform into a quadruplex structure. In one embodiment, the G content of the sequence of the first sequence segment of this aspect of the present invention is equal to or greater than 70%. In another embodiment, the G content may be equal to or greater than 75%. More specifically, in one embodiment, the first sequence segment may have a sequence based on GGGTGGGTGGGTGGGT [SEQ. ID. NO. 5] [“(GGGT)₄”]. This sequence can form into a quadruplex. However, it will be recognized by those of ordinary skill in the art that the first sequence segment for use in this aspect of the present invention does not have to include the exact (GGGT)₄ sequence. By being “based on” the sequence (GGGT)₄, those of ordinary skill in the art will recognize that substitutions and/or deletions may be made to this base sequence, so long as the resulting first sequence segment based on the (GGGT)₄ sequence remains able to conform into a quadruplex structure. For example, as described above, other sequences may form quadruplexes provided they include a guanine amount that is sufficient to form such quadruplexes. Further, the sequences do not have to be based on (GGGT)₄, as there are other formulas that the sequences may be based on. One example of such a formula is d(G₃+N₍₁₋₇₎G₃+N₍₁₋₇₎G₃+N₍₁₋₇₎+G₃).

Further, and still referring to FIGS. 4A and 4B, this aspect of the present invention provides two types of isothermal amplification: one is an exponential amplification (shown in FIG. 4A), and the other is a linear amplification (shown in FIG. 4B). In both types of amplification, the reaction includes a nucleic acid construct 29 in the form of a stem-loop with a sequence in the loop region 32 that is complementary to the target nucleic acid 24. The stem portion of the nucleic acid construct is formed by a dissociative-structure-forming sequence (e.g., a quadruplex-forming sequence) 30 duplexed with its complementary strand 34. As can be seen in FIG. 4A, the dissociative-structure-forming sequence 30 of the stem is positioned proximal to the 5′ end of the molecule. And in panel FIG. 4B, the dissociative-structure-forming sequence 30 of the stem is positioned proximal to the 3′ end of the molecule. As will be discussed below, these different positions result in exponential amplification versus linear amplification. The complementary strand 34 also provides a primer binding site (PBS) for the primer 20, which is another component of the assay (designated by the arrows in FIGS. 4A and 4B). The primer and stem-loop coexist metastably before adding a target nucleic acid. In other words, in the absence of target nucleic acid, the primer remains free because the primer binding site is duplexed with the dissociative-structure-forming sequence of the stem-loop. Once added to the reaction mixture, the target nucleic acid binds to the loop region of the construct and unfolds it, which releases the PBS for binding with the primer, thereby initiating the amplification reaction (as described above).

When the at least one primer is free in the reaction mixture including at least one nucleic acid construct and at least one target nucleic acid, the primer remains in a form that does not assume a dissociative structure (e.g., it does not spontaneously fold into a quadruplex). As described above, the primer has a similar sequence to the 5′ end of the stem-loop conformation of the nucleic acid construct. And, as described above, that 5′ end of the nucleic acid construct is designed to form into a dissociative structure. However, the primer is formed as a shortened or truncated version of the sequence that appears at the 5′ end of the nucleic acid construct. As a result, the primer will not, on its own, form a dissociative structure. Rather, any extension involving the primer needs to first occur before it can form a dissociative structure (as will be described in greater detail below).

As mentioned above, FIG. 4 depicts two versions of this amplification process. FIG. 4A shows exponential amplification of the nucleic acid construct 29, and FIG. 4B shows linear amplification of the nucleic acid construct 29. One difference between these versions is the location of the first sequence segment (i.e., dissociative-structure-forming sequence) and the third sequence segment (i.e., PBS) in the stem-loop constructs. In FIG. 4A, the dissociative-structure-forming sequences is proximal the 5′ end and the PBS is proximal the 3′end. In FIG. 4B, the orientation is opposite, with the PBS proximal the 5′ end and the dissociative-structure-forming sequence proximal the 3′ end.

Referring now to FIG. 4A, exponential amplification is as follows: The reaction mixture includes at least one primer(s) 20, at least one nucleic acid construct(s) 29 (e.g., in stem-loop form), and target nucleic acid(s) 24. Target nucleic acid 24 binds to the loop region 32 of the construct 29 and unfolds it, which frees the PBS 34 (i.e., third sequence segment) for binding (with a primer 20), and initiates the amplification reaction. In other words, the dissociative-structure-forming sequence included in the first sequence segment 30 of the nucleic acid construct 29 is a sequence such as would be used for a primer in DSPA. The third sequence segment 34 which is complementary to that first sequence segment 30 therefore has a sequence that can provide a primer binding site for any primer 20 having the sequence of the first sequence segment (or similar sequence).

In order to create the amplification reaction, then, the nucleic acid stem-loop construct 29 described above is placed in a reaction with target nucleic acid 24, and at least one primer 20 having a dissociative-structure-forming sequence similar to, or the same as, the dissociative-structure-forming sequence in the first sequence segment (although these primers are free in solution and are not part of the stem-loop described above). Further, the primers are generally a truncated version of the dissociative sequence portion of the first sequence segment, such that they do not spontaneously form a dissociative structure, such as a quadruplex.

When the target nucleic acid 24 binds to the loop region 32 of the stem-loop, the stem-loop is unfolded. This unfolding releases the third sequence segment 34 (having a PBS) from the first sequence segment 30. The first sequence segment 30 will then spontaneously form its dissociative structure 22 (such as a quadruplex), and the PBS 34 remains free for the primers 20 in the mixture to bind thereto and start an amplification reaction.

As the primer 20 attaches to the PBS 34, during the extension step, then, it amplifies the loop portion of the now-unfolded stem-loop construct. As this occurs, the target nucleic acid 24 will be released from the extending duplex of unfolded stem-loop structure and primer 20/extending sequence 36, thereby allowing the target nucleic acid 24 to be free for binding to another stem-loop nucleic acid construct 29. As extension continues, the extending sequence of nucleotides will confront the sequence of the stem-loop (i.e., the first sequence segment) in the form of its dissociative structure. And upon further extension, the activity of Taq polymerase will convert the first sequence segment back into a duplex. Once Taq subsequently leaves this duplex (after completing assembly of dNTPs in the duplex), the first sequence segment will again dissociate from its complementary strand, uptake K⁺, and form a dissociative structure (e.g., quadruplex). Meanwhile, the primer at the 5′ end of the extending sequence will also assume its dissociative structure. This allows a new primer to attach to the now freed primer binding site (PBS) to continue the amplification process.

Additionally, as shown in the last step of FIG. 4A, Taq must displace the target nucleic acid. However, this will not be a problem since it has already been shown that DSPA works isothermally at 70-75° C., which confirms that at these temperatures Taq unfolds DNA duplexes efficiently.

Referring now to FIG. 4B, a process for linear amplification will be described. As discussed above, the nucleic acid stem-loop construct 29 used in linear amplification, as shown in FIG. 4B, has an opposite configuration as compared to the nucleic acid stem-loop construct 29 of FIG. 4A. Referring to FIG. 4B, one can see that the dissociative-structure-forming portion (of the first sequence segment—30) of the nucleic acid stem-loop construct is located at the 3′end of the molecule, whereas the PBS 34 is located at the 5′ end of the molecule. This nucleic acid stem-loop construct is combined with primer(s) 20 (arrow) and target nucleic acid 24. Upon combination, the target nucleic acid 24 binds to the loop region 32 of the nucleic acid stem-loop construct 29, thereby unfolding the stem-loop, and unwinding the stem. As this occurs, the PBS 34 is freed from the dissociative-structure-forming sequence (first sequence segment) 30, and the dissociative-structure-forming sequence assumes its dissociative confirmation 22 (e.g., quadruplex). With the PBS 34 now freed, it can hybridize to the primer 20 in the reaction mixture. However, rather than extending the sequence in a direction that causes displacement of the target nucleic acid, extension proceeds in an opposite direction. The sequence of the third sequence segment 34 is chosen so that binding of the primer 20 followed by extension will cause successive guanine residues (e.g., two guanine residues) to be added to the primer sequence. This causes that primer to form its dissociative structure 22 (e.g., quadruplex). Once this structure has formed, the primer binding site is again opened for another primer to hybridize, extend, and the process repeats itself.

As described above, the primers used are of a sequence that does not initially form a dissociative structure (such as a quadruplex), but that will do so upon extension of the sequence during amplification (such as by the addition of guanine residues). One exemplary embodiment of such a primer sequence is GGG(2Ap)GGGTGGGTG [SEQ. ID. NO. 3] (G3T-ss13). Those of ordinary skill in the art will recognize that G3T-ss13 is merely an example of a sequence that can be used as a primer in this aspect of the present inventions, and that other sequences that are capable of forming dissociative structures upon extension may be used. Further, those of skill in the art will recognize that G3T-ss13 has a sequence based on (GGGT)₄. It is a truncated version of (GGGT)₄ and has a detectable label—2Ap (2-aminopurine)—incorporated in the sequence. Those of skill in the art will recognize that other labels may be used in dissociative sequences.

As a result, signal evolves after adding guanines to the primer (as mentioned above and as will be described in greater detail below, a detectable label may be included in the dissociative-structure-forming sequence, which becomes detectable once the dissociative sequence—such as a quadruplex—is formed). Due to the fact that high amounts of dNTP (˜0.5 mM) may inhibit Taq, the reaction shown in FIG. 4B, which only needs, for example, a two-guanine extension in the described embodiment, may allow very high concentrations of signal molecules to form, such that the signal may be detected by the unaided eye.

During the design of nucleic acid constructs of this aspect of the present invention, particular care should be taken to avoid misfolding of the stem-loop, via techniques that are well known to those of ordinary skill in the art. Avoidance of misfolding of the stem-loop will prevent mispriming in the absence of target nucleic acids. Further, while the system shown in FIG. 4B is a linear amplification pattern, the detection process will be accelerated by the fact that it requires only slight elongation (e.g., two-nucleotide elongation), after which the quadruplex (or other conformation) quickly dissociates. Since this mechanism does not require quadruplex replication, the appropriate ionic strength can be achieved by K⁺ ions alone, which will further accelerate signal amplification. Further, one can design several stem-loop molecules complementary to the target nucleic acid at different positions, to further accelerate signal amplification, as will be appreciated by those of ordinary skill in the art.

A problem that may arise during linear amplification, such as that shown in FIG. 4B, is “leakage.” “Leakage,” as referred to herein, is generally a problem of false positives that can occur due to the ability of the 3′ end of the stem loop probe to form a dissociative structure, such as a quadruplex. As has been described above, the stem of the stem loop probe includes (1) a 5′ segment complementary to a primer (the primer having the ability to form a structure such as a quadruplex), and (2) a 3′ segment that is complementary to the 5′ segment. As the 3′ segment is complementary to the 5′ segment, it (like the primer) also has the ability to form a dissociative structure such as a quadruplex. “Leakage” is the problem that occurs when that 3′ end (of the stem loop probe) forms into the quadruplex or other dissociative structure in the absence of any target nucleic acid. This spontaneous formation of the quadruplex then frees the 5′ end of the stem loop probe for binding of the primers (which include 2Ap), which then form quadruplex structures and the 2Ap can be detected. This results in a false positive, since these readings can occur in the absence of target nucleic acid.

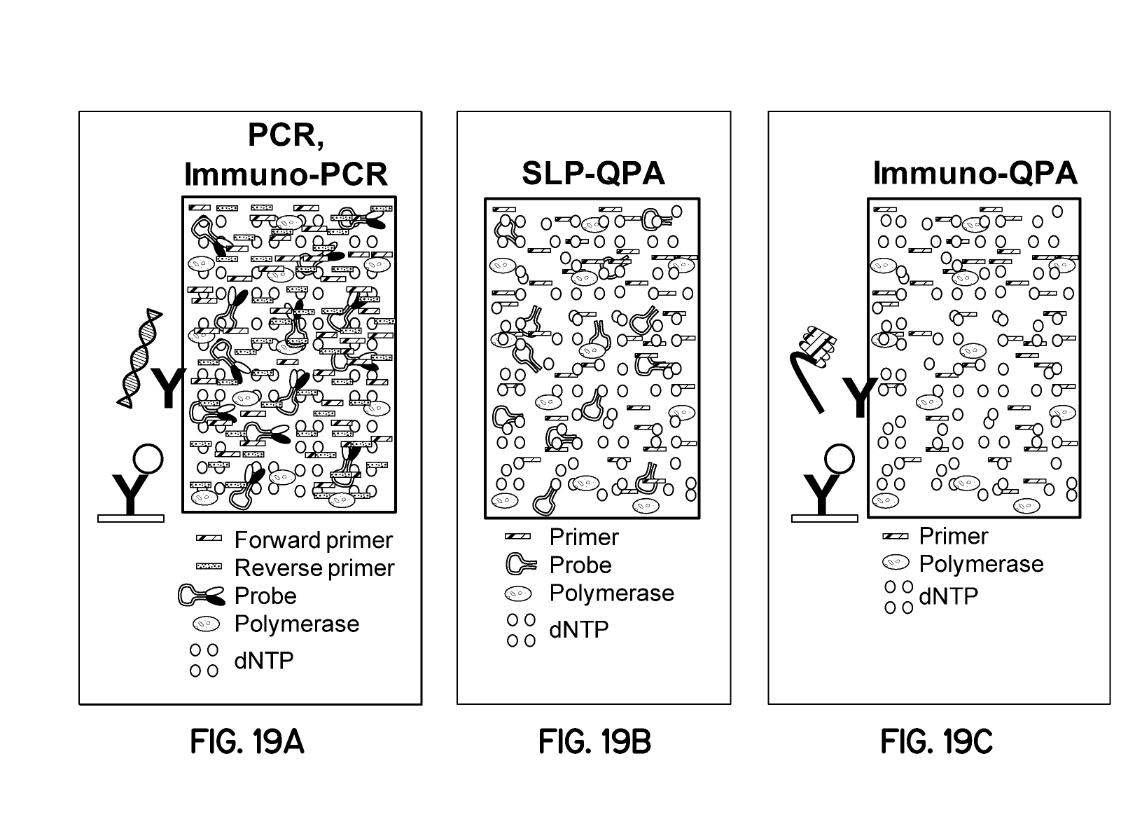

For example, FIG. 16 shows (1) in panel A, a sequence of an exemplary stem loop probe 29 (the 5′ to 3′ sequence at the bottom of panel A, with primer binder site underlined with hash-marked line 34, loop portion underlined 32, and dissociative-structure-forming portion underlined by dotted segment 30) as would be used in linear amplification (as shown in FIG. 4B) unfolded and bound to target nucleic acid 24 (underlined by a dashed line in panel A) and primer 20 (underlined by hash-marked line in panel A); and (2) in panels B and C, graphs demonstrating that the stem loop probe of this embodiment leaks since its 3′ end is able to form a dissociative structure, such as a quadruplex. The exemplary stem loop probe shown in FIG. 16 is 60 nt long, has a 15 by stem, and a 30 nt loop. And the target sequence is 33 bp. As can be seen from the graphs in panels B and C of FIG. 16, the presence of target nucleic acid in a reaction cause rapid primer binding, primer quadruplex formation, and detection of 2Ap (-□-). However, the reaction mixture including no target nucleic acid also results in an increase in primer binding, primer quadruplex formation, and detection of 2Ap (circled line -∘-).

In order to solve this problem of leakage (and false positives), in another aspect of the present invention, the 3′ end of the stem loop probe may include a substitution, such as a G->C substitution, which creates a CC mismatch between 3′ end of probe and 5′ end of target. This substitution prevents the 3′ end of the stem loop probe from forming a quadruplex (or other dissociative structure). While this is described above as a CC mismatch due to a G->C substitution, those of ordinary skill in the art will recognize that other methods may be used to achieve the result. For example, a G may be deleted. And so those of skill in the art will recognize that any sequence which remains complementary to the 5′ end of the stem loop probe, but which will not form a quadruplex or other dissociative structure, will suffice for this aspect of the present invention.

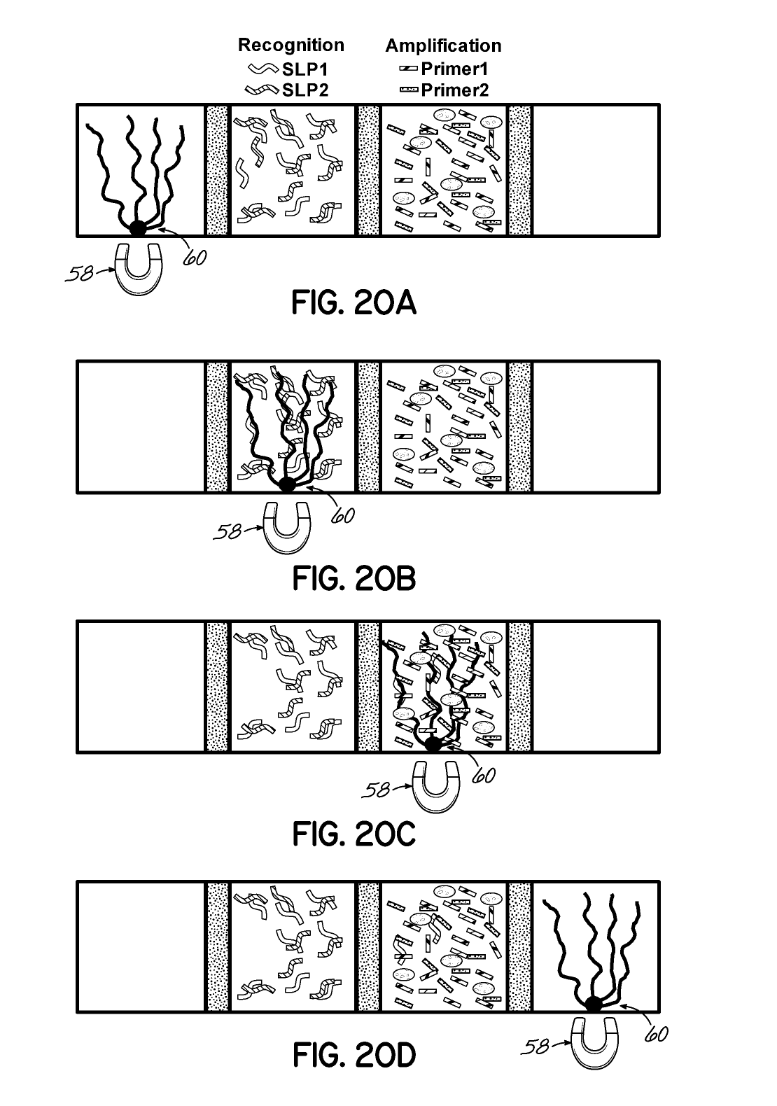

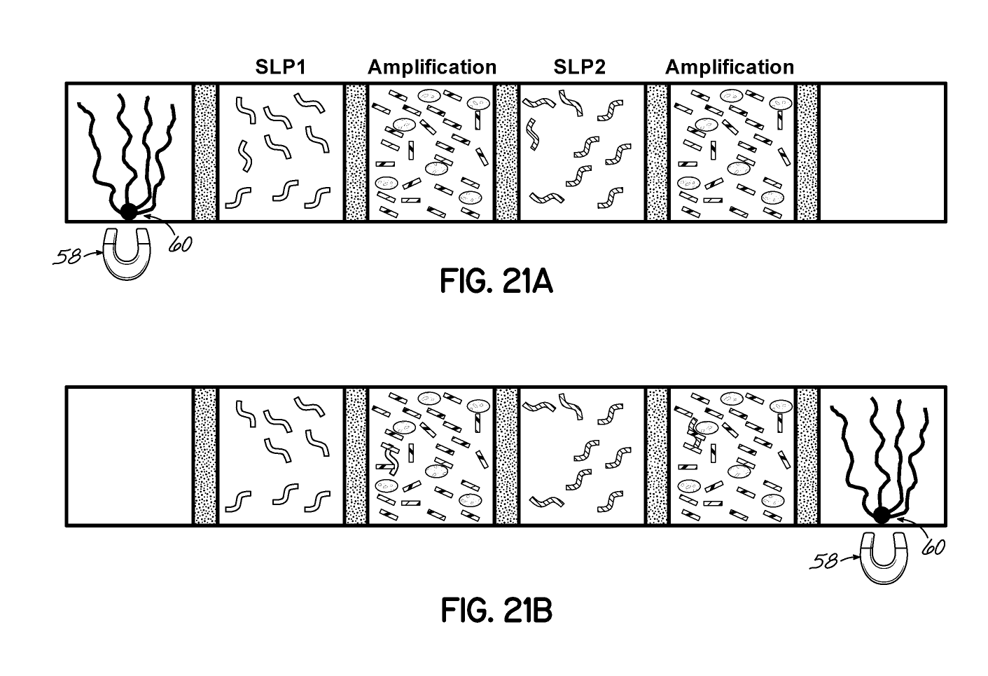

For example, FIG. 17, panel A, shows a sequence of an exemplary stem loop probe including a CG base pair (which previously had been a GC basepair from the version of the stem loop probe in FIG. 16), which does not destabilize the duplexed part of the stem-loop probe, but does cause a CC mismatch 38 between the target nucleic acid 24 and the 3′ end of stem loop probe that inhibits quadruplex (or other dissociative structure) formation. This prevents the 3′ end from forming a dissociative structure (such as a quadruplex). Also, the graphs of FIG. 17 panels B and C demonstrate that leakage (such as shown in FIG. 16) is reduced and eliminated by inhibiting the formation of the dissociative structure. In this exemplary embodiment, the stem loop probe is 60 nt long, has a 15 bp stem, and a 30 nt loop. The target sequence is 33 nt, but the combination of target with stem loop probe is such that there is one CC mismatch (at 5′ end of target/3′ end of probe). (Additionally then, there is a GG mismatch 40 at 5′ end of the probe/3′ end of the primer. However, the primer, incorporating 2Ap (shown by boldfaced “A”) is still able to form dissociative structure and release.) Note that in this exemplary embodiment, the initial slope of the line shown for “no target” (black line) in the graphs of panels B and C is about 11-fold less that the slope of the reaction including target nucleic acid (squared line). Further, as can be seen, the slope of the “no target” line (black line) is much less (closer to zero) than that shown in the graphs of FIG. 16. This demonstrates the result that leakage (such as shown in FIG. 16) is reduced and eliminated by inhibiting quadruplex formation (or other dissociative structure) at the 3′ end of the stem loop probe, such as by the G->C substitution described above. A schematic of this process (as embodied in the example of FIG. 17) is shown in FIG. 18.

As described above, the nucleic acid construct may include a label so that amplification may be detected (e.g., to thereby determine the presence of target nucleic acid). More specifically, in one embodiment, the first sequence segment of the nucleic acid construct and/or the primer may have a label incorporated therein. Such a label may be chosen from labels that are known to those of ordinary skill in the art. Such labels include, but are not limited to, fluorescent labels. And in a particular embodiment, such a label may include 2Ap.

The inclusion of a label in a dissociative sequence overcomes many of the previously described drawbacks of current systems. As described above, quantification methods are based on the fact that the amount of target nucleic acid produced and detected is directly proportional to the initial amount of sample DNA during the exponential growth phase. Since the fluorescence signal during the initial cycles is too weak to be distinguished from the background fluorescence (see the AB segment of FIG. 1) only a narrow window of the exponential growth phase is used for quantification (see the BC segment of FIG. 1). Thus, efficiency would be improved by reducing the background fluorescence (i.e., by the use of well-quenched probes before detection), and by a strong and immediate increase of fluorescence upon amplification, as well as by a longer exponential phase [as described in Edwards, K. J. et al. (2009) In Logan, J. et al. (eds.), Real-time PCR. Caister Academic Press, Norfolk, UK, pp. 85-93; Pfaffl, M. W. et al. (2009) In Logan, J. et al. (eds.), Real-time PCR. Caister Academic Press, Norfolk, UK, pp. 65-83].

As described above currently, four main probes are used to monitor real-time PCR [Lee, M. A., et al. (2009) In Logan, J. et al. (eds.), Real-time PCR. Caister Academic Press, Norfolk, UK, pp. 23-45]: (1) SYBR Green [Becker, A. et al. (1996) A quantitative method of determining initial amounts of DNA by polymerase chain reaction cycle titration using digital imaging and a novel DNA stain. Anal Biochem, 237, 204-207], (2) molecular beacons [Tyagi, S. et al. (1998) Multicolor molecular beacons for allele discrimination. Nature biotechnology, 16, 49-53; Tyagi, S. et al. (1996) Molecular beacons: probes that fluoresce upon hybridization. Nature biotechnology, 14, 303-308], (3) TaqMan® probes [Holland, P. M. et al. (1991) Detection of specific polymerase chain reaction product by utilizing the 5′----3′ exonuclease activity of Thermus aquaticus DNA polymerase. Proc Natl Acad Sci USA, 88, 7276-7280], and (4) Scorpion™ probes [Whitcombe, D. et al. (1999) Detection of PCR products using self-probing amplicons and fluorescence. Nat Biotechnol, 17, 804-807]. And, as described previously, there are drawbacks to each of these. SYBR Green is a dye that intercalates into double-stranded DNA nonspecifically resulting in fluorescence. Although SYBR Green is inexpensive, sensitive and easy to use, it also binds to any double-stranded DNA including nonspecific products or primer dimers.

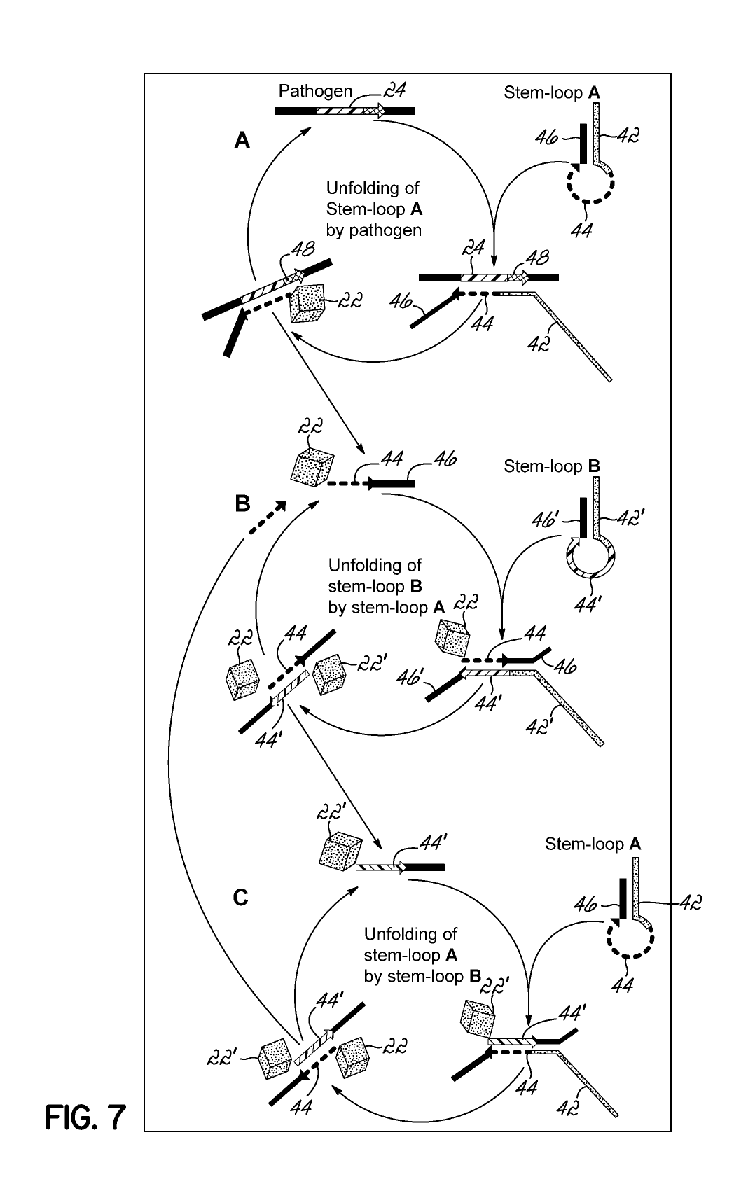

Referring to FIG. 6, panel A, molecular beacons are single-stranded oligonucleotide probes that form a hairpin-shaped stem-loop structure. The loop contains a probe sequence (dashed line segment, FIG. 6, panel A) that is complementary to a target sequence in the PCR product. The stem is formed by the annealing of complementary sequences that are located on either side of the probe sequence. A fluorophore (dotted circle) and quencher (lined circle) are covalently linked to the ends of the hairpin. Upon hybridization to a target sequence the fluorophore is separated from the quencher and fluorescence increases. Hybridization usually occurs after unfolding of the hairpin and product duplexes in the denaturation step of the next PCR cycle.

As described above, there are several disadvantages with molecular beacons. First, they require two bulky and costly tags (fluorophore and quencher). Second, the assay requires a separate probe for each template (i.e., mRNA), which dramatically increases the design effort and expense. Third, the mechanism uses separate binding sites for primer and probe sequences. This introduces another component (probe oligonucleotide) to an already complex reaction, and adds additional design limitations due to the need to avoid interactions between the probe and primers. Fourth, hybridization of the probe requires heating steps to unfold the product duplex and hairpin. Consequently, molecular beacons can't be used under isothermal conditions. Fifth, design of the probes requires considerable effort and knowledge of nucleic acid thermodynamics. And sixth, probe hybridization involves a bimolecular probe-primer system. This makes the reaction entropically unfavorable, slows down hybridization and complicates product detection at exponential growth. The hybridization is much faster and efficient with monomolecular probe-primer system [as described in Whitcombe, D. et al. (1999) Detection of PCR products using self-probing amplicons and fluorescence. Nat Biotechnol, 17, 804-807].

Referring now to FIG. 6, panel B, TaqMan® probes are single-stranded unstructured oligonucleotides designed to be complementary to a PCR product. They have a fluorophore attached to the 5′ end and a quencher coupled to the 3′ end. When the probes are free in solution, or hybridized to a target the proximity of the fluorophore and quencher molecules quenches the fluorescence. During PCR, when the polymerase replicates a template on which a TaqMan® probe is bound, the 5′-nuclease activity of the polymerase cleaves the probe. Upon cleavage, the fluorophore is released and fluorescence increases. The shortcomings listed above for molecular beacons hold true for TaqMan®. An additional disadvantage of TaqMan® probes is that they require the 5′-nuclease activity of the DNA polymerase used for PCR.

Referring now FIG. 6, panel C, Scorpion™ probes use a single oligonucleotide that consists of a hybridization probe (stem-loop structure similar to molecular beacons) and a primer (10) linked together via a non-amplifiable monomer (12). The hairpin loop contains a specific sequence that is complementary to the extension product of the primer (dashed line). After extension of the primer during the extension step of a PCR cycle, the specific probe sequence is able to hybridize to its complement within the extended portion when the complementary strands are separated during the denaturation step of the subsequent PCR cycle, and fluorescence will thus be increased (in the same manner as molecular beacons). Many of the shortcomings listed for molecular beacons hold true for Scorpion™ probes. First, they require two bulky and costly tags (fluorophore and quencher). Second, the assay requires a separate probe for each template (i.e., mRNA), which dramatically increases the design effort and expense. Third, the mechanism uses separate binding sites for primer and probe sequences. This introduces another component (probe oligonucleotide) to an already complex reaction, and adds additional design limitations due to the need to avoid interactions between the probe and primers. Fourth, hybridization of the probe requires heating steps to unfold the product duplex and hairpin. Consequently, Scorpion™ probes cannot be used under isothermal conditions. And fifth, design of the probes requires considerable effort and knowledge of nucleic acid thermodynamics.

Thus, in certain embodiments, the first sequence segment and/or the primer may include a sequence that is generally based on a sequence in the form of d(G₃₊N₁₋₇G₃₊N₁₋₇G₃₊N₁₋₇G₃) and include a label. In another embodiment, the first sequence segment and/or the primer may include a sequence that is generally based on the (GGGT)₄ sequence and includes a label such as 2Ap. And so, at least a portion of the first sequence segment and/or the primer may have a sequence based on 2Ap-G3T (GGG2ApGGGTGGGTGGG) [SEQ. ID. NO. 1]. However, it will be recognized by those of ordinary skill in the art that this sequence is not necessarily the entire sequence of the first sequence segment and/or the primer, merely that the first sequence segment and/or the primer may include the sequence based on 2Ap-G3T as a portion of the overall sequence of the first sequence segment and/or the primer.

In particular, in the illustration of FIG. 2, a primer that is a truncated version of (GGGT)₄ (a 13b primer in the illustrated embodiment) and incorporates 2Ap is used. In another particular embodiment, this primer has the sequence GGG(2Ap)GGGTGGGTGGG (2Ap-G3T—a.k.a. G3T-ss15) [SEQ. ID. NO. 1]. When this primer is not in the quadruplex conformation, fluorescence of 2Ap is quenched. In alternate embodiments, the primer may include different, albeit similar, sequences. For example, in one alternate embodiment, the primer may have the sequence GGG(2Ap)GGGTGGGTGG (G3T-ss14) [SEQ. ID. NO. 7]. And in another alternate embodiment, the primer may have the sequence GGG(2Ap)GGGTGGGTG (G3T-ss13—as in the illustrated embodiment) [SEQ. ID. NO. 3]. As can be seen in the top panel of FIG. 2, before elongation, the primers (here shown as a 13b primer e.g., GGG(2Ap)GGGTGGGTG [SEQ. ID. NO. 3]) form duplexes with the target sequence since they are missing a few guanine residues that would result in quadruplex formation. Elongation then begins, with the DNA polymerase adding dNTPs to the end of the primers (as shown in the second panel of FIG. 2). This elongation then eventually adds the length and/or guanine residues necessary to allow a quadruplex structure to be formed. Once this occurs (see the third panel of FIG. 2), the 5′-end of each product DNA is trapped in a quadruplex and its complementary sequence (the target DNA) is fully accessible to another incoming primer. And with the formation of the quadruplex, 2Ap is no longer quenched. In still further embodiments, the primers may have the sequence GG(2Ap)TGGTGTGGTTGG [SEQ. ID. NO. 8] or may have the sequence GGTTGG(2Ap)GTGGTTGG [SEQ. ID. NO. 9].

Further, since the conformation taken on by the first sequence segment and/or the primer sequence (such as a quadruplex) is more stable than its corresponding duplex, unfolding of the duplex or release of target for the incoming primers can occur without the need of substantial temperature change or any temperature change. In other words, in standard PCR, following the extension step, the DNA is in a duplex form. The next cycle then begins by raising the temperature to a point that the double-stranded DNA again denatures (i.e., separates into single strands). This is necessary in order to provide the separated sense and antisense single-stranded DNA strands for primer binding (to each of the strands), followed by elongation during the next extension step (once the temperature of the reaction is reduced). However, by using first sequence segments and/or primers based on a dissociative-structure-forming sequence, such as the (GGGT)₄ sequence, the primers plus extending nucleotides that are added during the extension step, and the first sequence segments, naturally conform into a structure such as a quadruplex. As this occurs, the primer (e.g., forming the quadruplex structure) naturally separates from the target DNA sequence complementary to the primer, thereby leaving the target region complementary to the primer exposed in single-stranded form for binding of the next primer (as can be seen in FIG. 4A, this occurs in both strands). This occurs without requiring raising of the temperature to denature the strands from one another. Thus, amplification can proceed under isothermal conditions. And so, the isothermal DNA amplification provided by the present invention does not require expensive instrumentation for thermocycling and may allow DNA amplification in the field and at point-of-care. And, the product yield in this isothermal system may be characterized using real-time fluorescence measurements of 2Ap incorporated within DSPA primers.

Another aspect of the present invention provides protein-free dissociative-structure-based amplification.

As described in the Background, PCR, which depends on the enzymatic activity of DNA polymerases, is not ideally suited for point-of-care use. Polymerization-based amplification yields a macroscopically observable polymer, visible to the unaided eye [Hansen, R. R., Johnson, L. M. and Bowman, C. N. (2009) Visual, base-specific detection of nucleic acid hybridization using polymerization-based amplification. Analytical biochemistry, 386, 285-287]. However, it still depends on DNA polymerization for coupling biotinylated dNTPs to DNA hybrids, and requires 0.5 nM or higher target concentrations. Surface plasmon resonance coupled with interferometry [Kim, D. K., Kerman, K., Saito, M., Sathuluri, R. R., Endo, T., Yamamura, S., Kwon, Y. S. and Tamiya, E. (2007) Label-free DNA biosensor based on localized surface plasmon resonance coupled with interferometry. Analytical chemistry, 79, 1855-1864] detects 10 μM target but requires instrumentation unsuitable for POC diagnostics. Gold nanoparticles can be visualized with the unaided eye at high pM to nM target concentrations [Thaxton, C. S., Georganopoulou, D. G. and Mirkin, C. A. (2006) Gold nanoparticle probes for the detection of nucleic acid targets. Clinica chimica acta; international journal of clinical chemistry, 363, 120-126]. However, to increase sensitivity further, they must be coupled with PCR or other specialized detection platforms. A sandwich-type binding assay is able to detect 60 fmol target DNA, however it depends on biotinylated capture oligonucleotides, repeated washing steps and additional liposome components [Zimmerman, L. B., Lee, K. D. and Meyerhoff, M. E. (2010) Visual detection of single-stranded target DNA using pyrroloquinoline-quinone-loaded liposomes as a tracer. Analytical biochemistry, 401, 182-187]. Hybridization chain reaction [Yin, P., Choi, H. M., Calvert, C. R. and Pierce, N. A. (2008) Programming biomolecular self-assembly pathways. Nature, 451, 318-322] and entropy-driven signal amplification [Zhang, D. Y., Turberfield, A. J., Yurke, B. and Winfree, E. (2007) Engineering entropy-driven reactions and networks catalyzed by DNA. Science (New York, N.Y, 318, 1121-1125] do not require the use of specific detection platforms, and are based on autocatalytic reactions between DNA oligonucleotides in solution. However, both methods use complicated reactions and detection mechanisms. In addition, these methods suffer from low sensitivity due to significant levels of spontaneous autocatalysis even in the absence of target molecules.

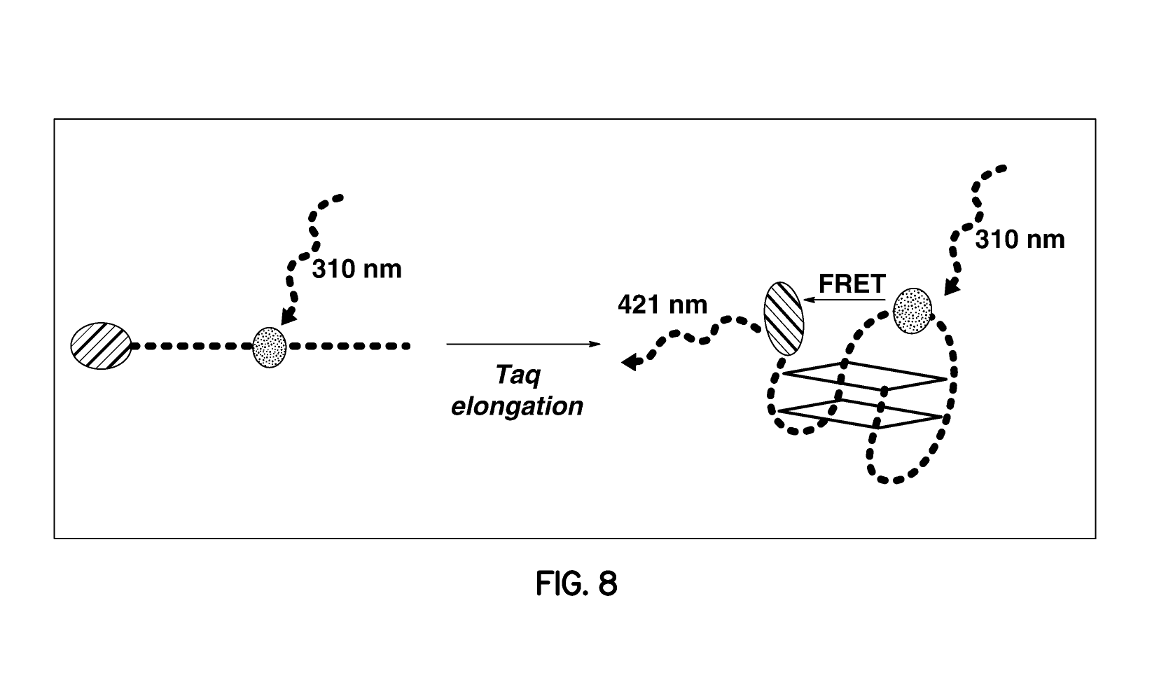

In general, and referring now to FIG. 7, another aspect of the present invention overcomes these drawbacks. This aspect provides a mixture of nucleic acid constructs including at least one first nucleic acid construct and at least one second nucleic acid construct. The at least one first nucleic acid construct and at least one second nucleic acid construct are designed such that they work in concert to provide an amplification reaction that can identify a target nucleic acid sequence (e.g., a DNA sequence) without the use of enzymes as in standard PCR. The reaction in this aspect of the present invention may also proceed isothermally.

To that end, the first nucleic acid construct (designated “stem-loop A” in FIG. 7) includes a first sequence segment 42 (dotted segment) and a second sequence segment 44 (dashed line), wherein at least a portion of the first sequence segment includes a sequence adapted to conform into a structure 22 that dissociates from a complementary strand of a DNA duplex, and wherein at least a portion of the second sequence segment includes a sequence that is complementary to a target nucleic acid sequence 24. And, the second nucleic acid construct (designated “stem-loop B” in FIG. 7) includes a first sequence segment 42′ (dotted segment) and a second sequence segment 44′, wherein at least a portion of the first sequence segment includes a sequence adapted to conform into a structure 22′ that dissociates from a complementary strand of a DNA duplex, and wherein at least a portion of the second sequence segment includes a sequence that is substantially similar to the target nucleic acid such that the second sequence segment 44′ of the second nucleic acid construct (“stem-loop B”) can bind with the second sequence segment 44 of the first nucleic acid construct (“stem-loop A”).

Like the nucleic acid construct for isothermal amplification and identification described above, each of the first and second nucleic acid constructs in this aspect of the present invention may be provided in the form of stem-loop constructs. In each of the stem-loop constructs, the portion of the sequence which includes a dissociative-structure-forming sequence provides a segment of the stem (being duplexed with a complementary sequence 46, 46′—black). A primary portion of the loop of the first nucleic acid construct includes a sequence that is complementary to the target nucleic acid, and a primary portion of the loop of the second nucleic acid construct includes a sequence that is substantially the same as the target nucleic acid sequence.

When the first and second nucleic acid constructs of this aspect of the present invention are combined with target nucleic acid, the target nucleic acid hybridizes with the loop segment of the first nucleic acid construct. This unfolds the stem-loop of the first nucleic acid construct, thereby unwinding the stem. Once unwound, the sequence of the now free dissociative-structure-forming sequence (i.e., the first sequence segment) forms its dissociative structure (hash-marked box—e.g., a quadruplex). As a result, the DNA duplex between the loop segment and the target nucleic acid is destabilized and the complex quickly dissociates. The released target binds to another first nucleic acid stem-loop construct and repeats the same cycle.

Meanwhile, the denatured first nucleic acid construct, now having a dissociative structure 22 at its 5′ end (dotted box), binds to the stem-loop of the second nucleic acid construct [with hybridization between the second sequence segment 44 of the first nucleic acid construct and the loop segment (second sequence segment 44′) of the second nucleic acid construct]. This induces a similar unwinding/dissociation process in the second nucleic acid construct. Once unwound, the first sequence section of the second nucleic acid construct forms its dissociative structure 22′ (dotted box—e.g., a quadruplex). As a result, the DNA duplex between the first and second nucleic acid constructs is destabilized, and the two separate. The released second nucleic acid construct now binds to and unfolds stem-loop of another first nucleic acid construct. At this point, the reaction becomes autocatalytic, i.e., the product of each cycle serves as the catalyst for the subsequent cycles. Thus, amplification occurs in the absence of any standard DNA polymerases, and can proceed isothermally.

A key feature of the system is that the large potential energy of quadruplex formation is captured in DNA duplexes with significantly lower free energies, which is achieved by pre-forming the duplexes in the presence of Cs⁺ and adding quadruplex-forming K⁺ afterward. Thus, after adding K⁺, ions quadruplex formation becomes thermodynamically favorable but kinetically trapped. In other words, K⁺ ions bring the stem-loops to a metastable condition similar to a chain of dominos in the upright position. Adding the target nucleic acid results in the exponential domino effect.

Initially, the target nucleic acid 24 hybridizes with the first nucleic acid construct (stem-loop A) and unwinds the stem (as shown in FIG. 7, panel A). Target nucleic acid 24 hybridizes with the second sequence segment 44 and a few guanines from the first sequence segment 42 and therefore the target nucleic acid 24 should contain a few cytidines at the 3′-end 48 (cross-hatched arrow). This can be accomplished by adding cytidines, where necessary, to the sequence of target DNA. While this may be useful in a laboratory setting, it is not as useful in point-of-care analysis. And so, alternatively, a target segment may be chosen that already has the necessary cytidines (repeating cytidines are common in nucleic acid sequences, as is known to those of ordinary skill in the art). Thus, the newly formed hybrid includes terminal guanines of the dissociative-structure-forming sequence, which is not enough to prevent formation of the dissociative structure (e.g., quadruplex) at the reaction temperature. As a result, the DNA duplex is destabilized and the complex quickly dissociates. The dissociative structure 22 (e.g., quadruplex) formation is accompanied by contraction of the loop segment by a few terminal guanines, which inhibits the reverse reaction between newly dissociated strands. Released target binds to another stem-loop A (see FIG. 7, panel A) and repeats the same cycle, while denatured A binds to the stem-loop B and induces a similar unwinding/dissociation process (see FIG. 7, panel B), which is followed by unfolding of stem-loop A by denatured B (see FIG. 7, panel C). At this point, the reaction becomes autocatalytic, i.e., the product of each cycle serves as the catalyst for the subsequent cycles.

In order to design a successful non-enzymatic signal amplification system, the length of the nucleic acid constructs and experimental conditions should be selected carefully and reactions should be conducted at the appropriate temperature such that: (i) the stem-loop constructs are folded; (ii) the target should be able to bind and unfold the stem-loop; (iii) dissociative structure formation at the end of the stem-loop should be favorable; and (iv) the bimolecular complex between target and hairpin should dissociate itself.

Further, as described above, a drawback of current RT-PCR-specific quantification systems is that they use FRET-based applications (Förster Resonance Energy Transfer), which require costly synthesis and considerable effort to design a sensitive probe. As is known to those of ordinary skill in the art, FRET is a mechanism describing energy transfer between two chromophores. A donor chromophore, initially in its electronic excited state, may transfer energy to an acceptor chromophore (in proximity, typically less than 10 nm) through nonradiative dipole-dipole coupling. Unfortunately, the processes currently used require multiple probes for multiple targets (i.e., one probe for each target), which greatly increases materials, time, and expense.

Another disadvantage of the current RT-PCR detection mechanisms is that two separate functions, recognition and detection, are combined within a probe (see the discussion of currently used process in the Background). The bifunctional nature of the probes requires that the fluorophore-quencher pair be attached to each DNA probe sequence, which makes quantification impractical when several targets are tested. As shown in FIGS. 3 and 4, DSPA separates these two functions, which allows using the same reporter molecule for different targets.

More specifically, DSPA described in FIG. 4 consist of the following steps: (i) template recognition by a stem-loop probe, which is accompanied by release of the PBS; (ii) priming; and (iii) primer elongation, which is accompanied by light emission. By comparison, traditional RT-PCR consists of: (i) template recognition by primers; (ii) amplification; and (iii) recognition of amplicons by a probe, which is accompanied by fluorescence reporting. Thus, in traditional RT-PCR recognition happens twice, while in DSPA it only occurs once. Therefore, to perform 96-well quantification using molecular beacons, for instance, it is necessary that the same fluorophore-quencher pair be attached to 96 different probes, while in DSPA one can use a single primer (e.g., GCGC-G3T-ss17) [SEQ. ID. NO. 10] for all 96 targets. Thus, DSPA uses intrinsic fluorescence of primers and quantifies different templates with the same probe.

Multiplexing, or detecting more than one target in the same tube, requires several primers with (i) similar thermal stabilities, but with high selectivity to their matching binding sites, and (ii) distinct fluorescence properties for each probe. Since DSPA primers are limited to specific guanine-rich sequences and there are only a limited number of intrinsically fluorescent nucleotide analogs, the ability of DSPA to be applied to multiplexing is not obvious.

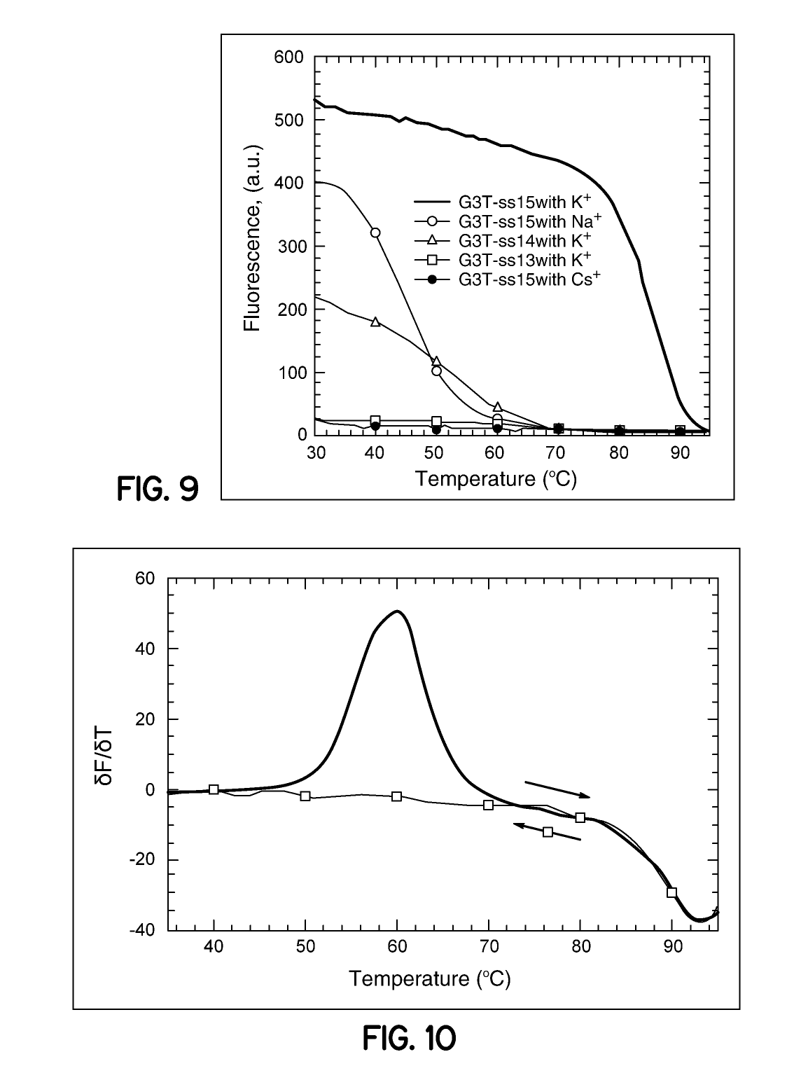

Thus, another aspect of the present invention provides FRET-based DSPA detection, which increases the multiplex capability of DSPA. A fluorescent nucleotide donor will be placed internally and a fluorescent acceptor will be attached at 5′-end of a DSPA primer (FIG. 8). The fluorescent acceptor may be positioned proximal to the 5′ end of the primer. The fluorescent nucleotide donor may be 2Ap. And the fluorescent acceptor may be Alexa405. The fluorescence emission peak of 2Ap overlaps the excitation peak of attached Alexa405. Such a double labeled DNA is commercially available from TriLink Biotechnologies. No fluorescence signal will be observed before quadruplex formation since 2Ap is quenched by adjacent nucleotides. Upon quadruplex formation 2Ap emits light at 370 nm and energy from 2Ap is transferred to Alexa405 resulting in an emission signal at 420 nm. The attachment will increase cost of the synthesis, but since one particular DSPA primer can be used to detect different nucleic acid targets, the overall cost will still be significantly lower than current RT-PCR approach (including multiple probes for multiple targets). Additional 2Ap-based FRET probes may include using Alexa350 (Ex343, Em442) as 5′-end attachment, while pteridine will be coupled with Alexa430 (Ex434, Em541) or Alexa488 (Ex495, Em519). Other fluorescent nucleotides may include pteridine analogs: 3-methyl isoxanthopterin (3MI) (Ex348, Em431), 6-methylisoxanthopterin (6MI) (Ex340, Em430) and (4-amino-6-methyl-8-(2¢-deoxy-â-D-ribofuranosyl)-7(8H)-pteridone (6AMP) (Ex330, Em435).

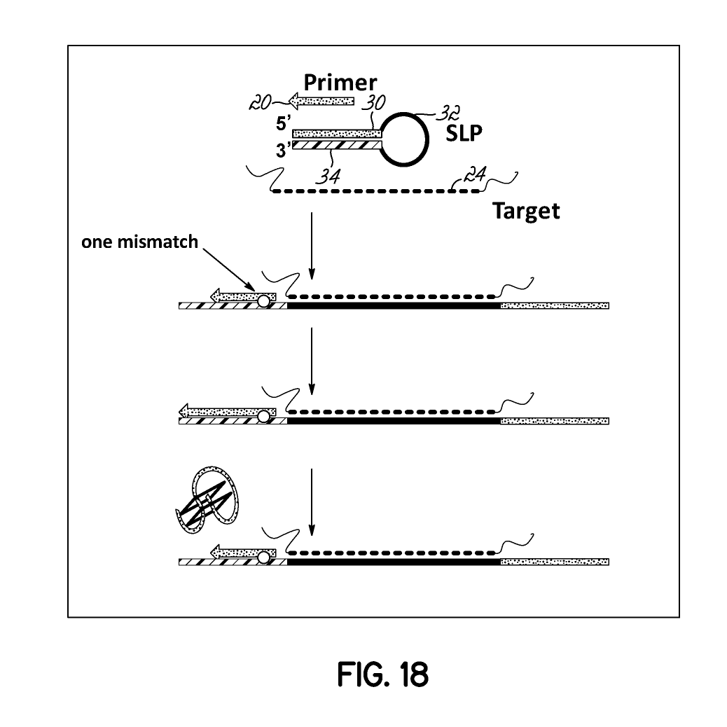

In another aspect of the invention, and referring now to FIG. 15, a nucleic acid construct may be provided that including multiple sequence segments, each having a detectable label, that allows for amplification of the signal generated.

More specifically, the nucleic acid construct may include (1) a first sequence strand, and (2) plurality of nucleotide segments, wherein the plurality of nucleotide segments each include a sequence that is complementary to at least a portion of the sequence of the first sequence strand. As a result, the plurality of nucleotide segments can act as a segmented version of a complementary strand, and, at least initially, retain the first sequence strand in a pseudo-duplex form (as shown in the top panel of FIG. 15).

The first sequence strand of nucleotides includes from the 5′ to the 3′ end: (1) a first segment having a sequence of nucleotides complementary to a target nucleic acid, and (2) a plurality of segments, each of said plurality of segments having a detectable label. Each of the plurality of segments is adapted to conform into a conformation having a free energy with more favorable thermodynamics than a corresponding B-DNA duplex. For example, each of the plurality of segments may be adapted to conform into a quadruplex. Such formation of non-B-DNA duplex structures and their use in accordance with the principles of the present invention is discussed at length above.

As described above, the plurality of segments (numbered 1, 2, and 3 in the first panel of FIG. 15) initially retains the first sequence strand in a pseudo-duplex form. This is accomplished because each of the plurality of nucleotide segments includes a sequence that is complementary to either (1) a sequence spanning the first segment and one of the plurality of segments of the first sequence strand, or (2) at least two of the plurality of segments of the first sequence strand. As can be seen from FIG. 15 (and particularly the top panel thereof), the segment numbered “1” includes a portion complementary to a portion of the first sequence strand that is complementary to target DNA, and a portion complementary to a portion of the first labeled sequence. The segment numbered “2” includes a portion complementary to a portion of the first labeled sequence, and a portion complementary to a portion of the second labeled sequence. And, the segment numbered “3” includes a portion complementary to a portion of the second labeled sequence, and a portion complementary to a portion of the third labeled sequence.

Thus, in operation (and still referring to FIG. 15), the construct in this illustrated embodiment may include three segments 48, 50, 52 having a labeled sequence capable of conforming into a non-B-DNA duplex structure. It will be recognized by those of skill in the art that any number of such segments is possible. In a particular embodiment, the segments 48, 50, 52 may include a sequence such as GGGNGGGNGGGNGGG [SEQ. ID. NO. 11] (where “N” represents fluorescence nucleotides such as 2Ap or 6MI). Such a sequence is merely exemplary as other sequences may be used. Further it is not necessary that each of the segments include the same sequence.

The segments 48, 50, 52 may be connected to each other with a few nucleotides (Ts or Cs) 54 hybridized to three (or more) short segments (separate black segments 1, 2, 3) as shown in the illustrated embodiment. This prevents quadruplex (or other non-B-DNA duplex) formation before hybridization with target nucleic acid. When target nucleic acid 24 hybridizes with its complementary part 56, segment 1 is displaced. This is followed by first quadruplex 22 (or other non-B-DNA duplex) formation, which in turn destabilizes next bimolecular duplex (at segment 2) and so on (i.e., at segment 3, and any other further segments). As a result one can have many signals per construct. In alternate embodiments, one end of the construct (e.g., the left end as shown in FIG. 15) can be attached to a solid surface in a DNA chip, which would allow massive multiplexing.

DSPA results in higher specificity. To produce a false signal, non-specific priming alone is not enough. The non-specifically bound primer would have to bind at cytidine-tracts, which further decreases the possibility of a false signal. Since linear DSPA requires only dGTP and exponential DSPA can be performed in the presence of dGTP and dCTP, non-specific replication could be inhibited by using an incomplete set of dNTPs.

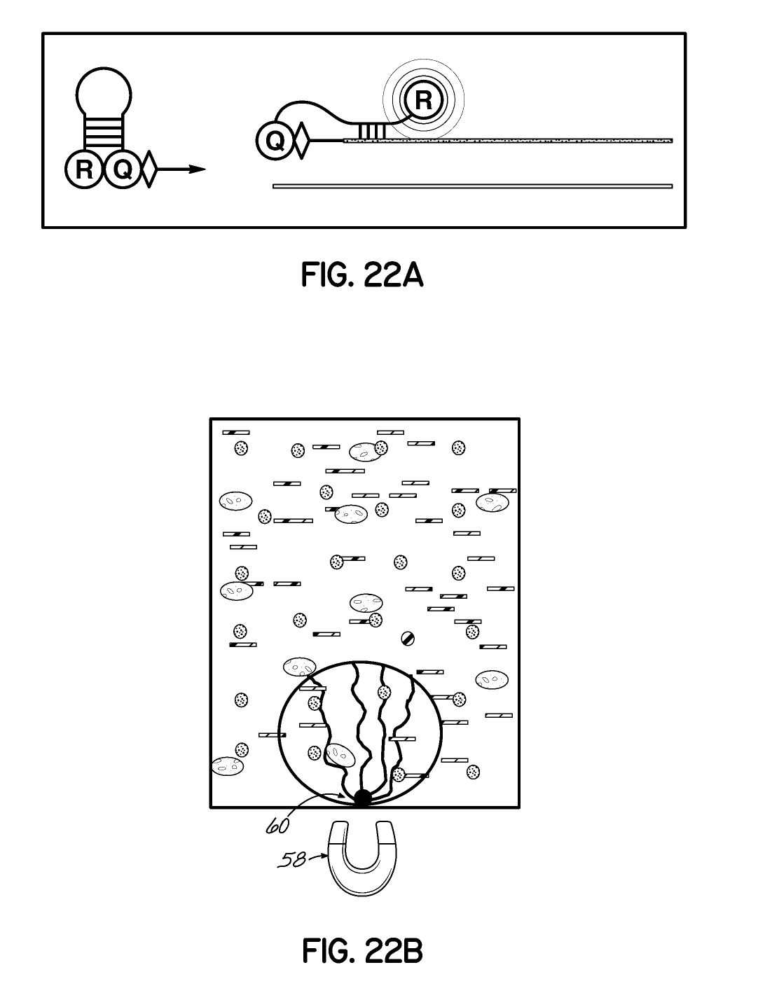

Referring now to FIGS. 19A-19C, one particular advantage of DSPA is shown: DSPA results in a simplified reaction mix as compared to traditional PCR. It also results in a simplified reaction mixture as compared to immune-PCR (an antigen detection system using PCR in which a specific DNA molecule is used as the marker—as described in, for example, Sano et al., Immuno-PCR: a very sensitive antigen detection by means of specific antibody-DNA conjugates, Science, 258 (5079), Oct. 2, 1992, pp. 120-122, incorporated by reference herein in its entirety). Referring particularly to FIG. 19A, one can see a typical PCR reaction as including forward primers, reverse primers, probes, polymerases, and dNTP's. Referring to FIG. 19B, one can see that the reaction mixture for DSPA including stem loop probes includes a single primer, the stem loop probe, polymerase, and dNTP's. And finally, referring to FIG. 19C, immuno-DSPA provides a reaction mixture only including primers, polymerases, and dNTP's. (One of ordinary skill in the art will note that FIGS. 19B and 19C refer to “QPA”—a.k.a. quadruplex priming amplification; however, as discussed above, the primers do not necessarily need to form into quadruplexes, as will be appreciated by those of ordinary skill in the art, but only need to form into any structure that dissociates from a complementary sequence, i.e., DSPA.)

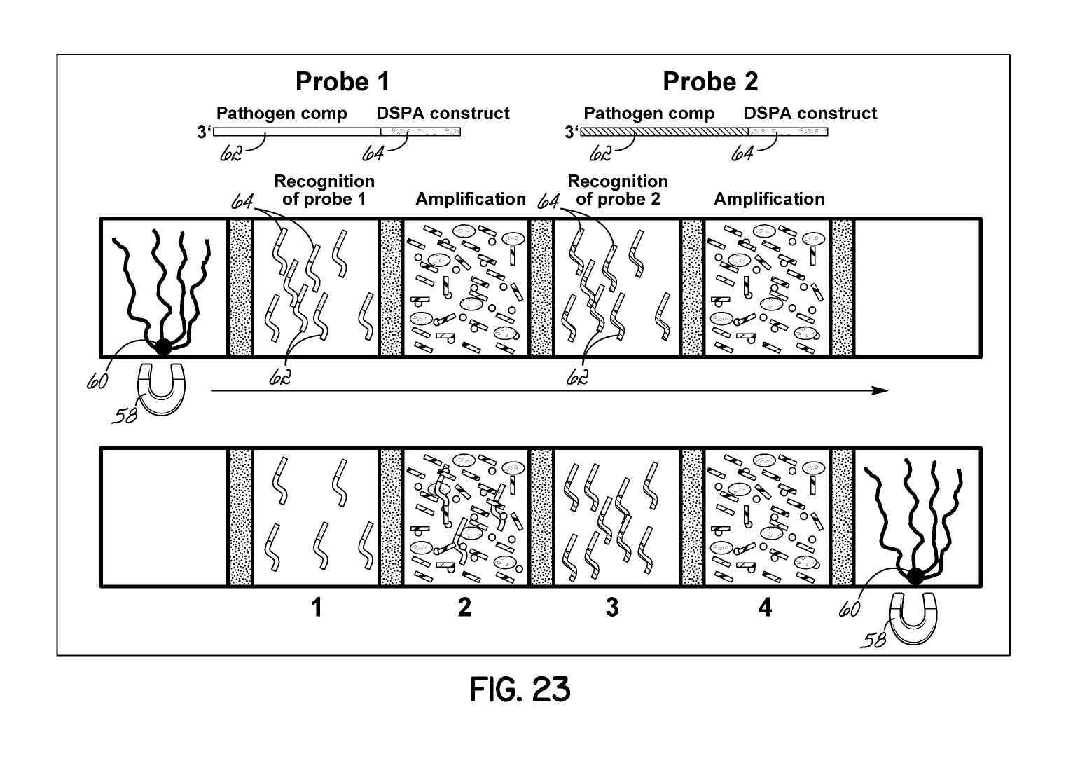

Another advantage of DSPA is shown in FIGS. 20A-D. As described previously, one of the major disadvantages of current RT-PCR detection mechanisms is that two separate functions, recognition and signal production, are combined within a probe. This requires the presence of primers and probes in the same solution, which complicates the reaction (as is shown in FIG. 19A, discussed above). In DSPA, however, these two functions are separated, which allows one to provide recognition and signal amplification in different solutions (see FIGS. 20A-D). As a result, the reactions are less complicated.

Further, in another embodiment of the present invention, a magnetic force or magnetic field 58 may be used to move any target nucleic acid through the solution (or sequentially through different reaction solutions—or steps of a reaction), by having nucleic acid adsorbed onto the surface of metal beads 60. One such type of bead is GeneCatcher™ Magnetic Beads (commercially available from Invitrogen, Carlsbad, Calif.). This allows for a further simplified yet effective reaction that lends itself to use at point-of-care. As has been described above, one benefit of DSPA is that it allows for nucleic acid-based detection at point-of-care or in settings where little resources are available. Current nucleic acid-based detection systems, such as quantitative PCR, are attractive technologies because of their sensitivity and specificity. However, the effectiveness of a typical PCR reaction is dependent upon the quality and quantity of the nucleic acid template, and the absence of interferents (e.g., carbohydrates, proteins, and lipids—which have all been shown to inhibit PCR and product false negatives). To minimize these false negatives and thereby maximize the efficiency of nucleic acid-based diagnostics, nucleic acids are often extracted and concentrated into an interferent-free buffer prior to testing. The methods used to do this are highly effective, but are time-consuming and often require the use of toxic organic chemicals. Other solid phase extraction kits are commercially available to purify DNA or RNA from patient samples, however, many of these kits rely on selective nucleic acid binding to silicone-coated surfaces in the presence of materials such as ethanol and guanidinium thiocyanate. Such kits are not cost-effective for low resource use and often require the use of specialized laboratory equipment and trained technicians, which decrease the effectiveness of their use as point-of-care technologies.

Thus, one embodiment of the present invention may include a reaction vessel including one or more defined sections, with a particular reaction mixture or part of a reaction mixture (e.g., including one or more components of a reaction mixture—primers, etc.) in different sections of the vessel. For example, referring to FIGS. 20A-D, a reaction vessel (such as a cassette) may include a chamber including a solution having stem loop primers (i.e., the second panel of FIGS. 20A-D) and a section including a solution having amplification primers (see the third panel of FIGS. 20A-D). With any target nucleic acid associated with metallic beads, (such as by being adsorbed onto the surface thereof—see the left-most panel of FIG. 20A), a magnetic field may then be moved along the cassette in order to move the metal bead and thus the nucleic acid target sequentially through the various solutions (see the magnet, representing magnetic field moving from panel to panel in FIGS. 20A-D). The general use of such a magnetic field to move metal beads with adsorbed nucleic acid through such cassettes is described in Bordelon et al., Development of a Low Resource RNA Extraction Cassette based on Surface Tension Valves, Applied Materials and Interfaces, 2011, 3, 2161-2168, which is incorporated by reference herein in its entirety.

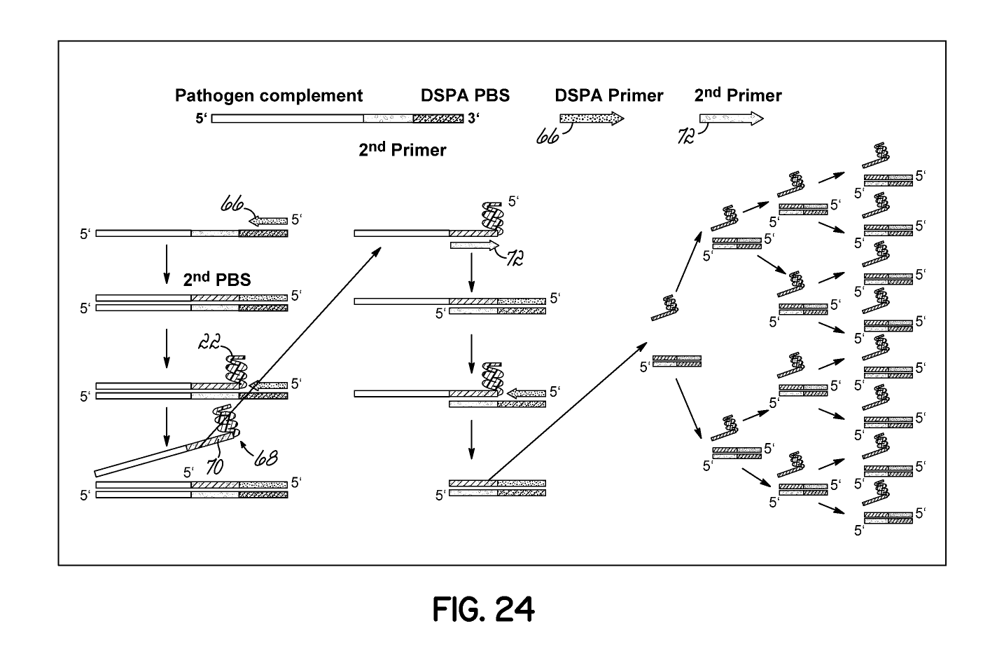

Referring to FIGS. 21A and 21B, the universal nature of the primer probe in DSPA and its use in multi-well diagnostics is shown. As described above, the bifunctional nature of RT-PCR probes requires that a fluorophore-quencher pair be attached to each DNA probe sequence, which makes quantification impractical when several targets are tested. Since DSPA separates these two functions, the same reporter molecule can be used in multiwell diagnostics. And again, as shown in FIGS. 21A and 21B, a magnetic field may be used to move any target nucleic acid attached to metal beads through cassettes that include separated segments having various reaction mixtures (e.g., a first stem loop primer probe segment, an amplification segment, a second stem loop probe segment, and a second amplification segment).

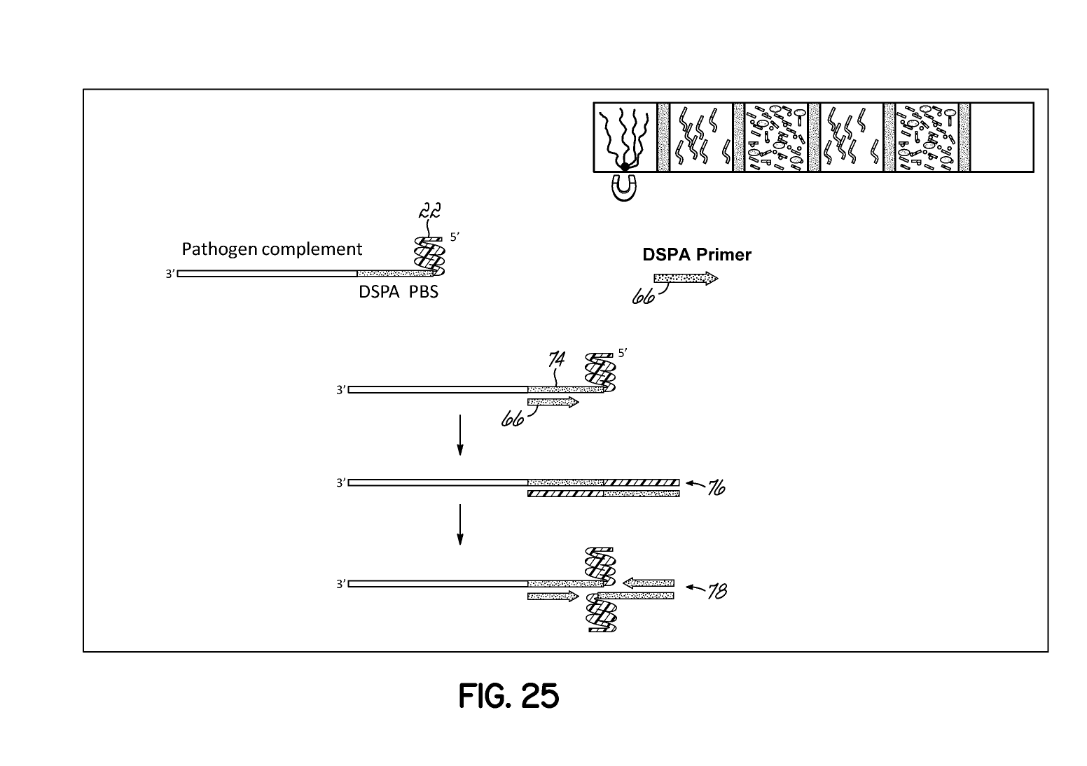

FIGS. 22A and 22B show the monomolecular nature of detection. As described above, Scorpion probes are one commonly used probe today. Scorpions use a single oligonucleotide that consists of a hybridization probe and a primer linked together via a non-amplifiable monomer. A hairpin loop contains a specific sequence that is complementary to the extension product of the primer. After replication then, the probe is covalently attached to the amplicon, which makes signal generation a monomolecular process. While this allows faster and earlier detection, Scorpions are complicated molecules having three attached modifications.

In contrast, signal generation in DSPA is not only a monomolecular reaction, but priming and probing is performed by the same oligonucleotide. Thus, DSPA allows simple detection of the very first amplicons.

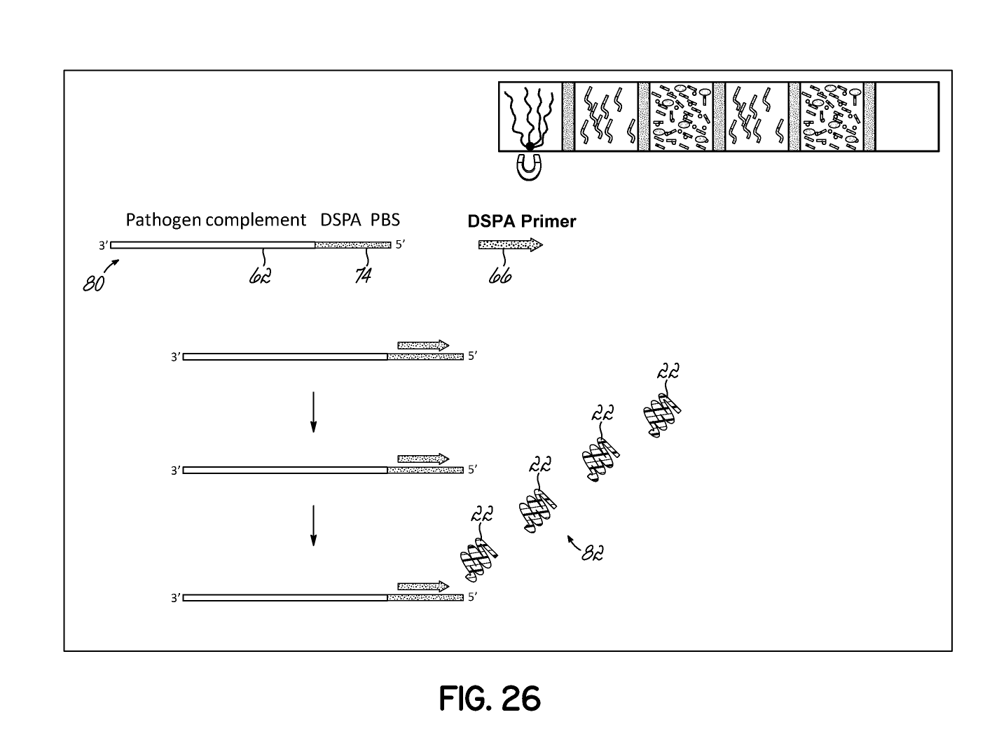

Further embodiments of the use of DPSA principles in multi-well diagnostics are as follows: Referring to FIG. 23, an embodiment for amplification and detection using two primers (a first primer being a DSPA primer and a second primer being a non-DSPA primer) and the use of a solid support (e.g., magnetic beads) in a multi-chambered housing is shown. Isothermal and exponential amplification can be conducted using this first primer (DSPA primer) in combination with the second primer.

And, in the illustrated embodiment of FIG. 23, the assay can be used to assess the presence of multiple targets. To that end, probes (“Probe 1” and “Probe 2”) specific for two different disease targets are shown at the top of the figure. Each of those probes includes (1) a segment (labeled “pathogen comp” in the figure) that is complementary to a sequence from the target nucleic acid, and (2) a segment including a sequence that can form a dissociative structure—or is capable of forming a dissociative structure upon elongation (labeled “DSPA construct” in the figure). The panels showing the multi-chambered housing in the figure show the sample chambers before (top) and after (bottom) movement of the sample through the chambers. (Washing steps are not shown in FIG. 23—such washing techniques are well known to those of ordinary skill in the art.)

Like the embodiment shown above with respect to FIGS. 20A-D, in the illustrated embodiment of FIG. 23 a magnetic force or magnetic field 58 may be used to move any nucleic acid (perhaps including a target nucleic acid sequence) through the solution (or sequentially through different reaction solutions—or steps of a reaction), by having nucleic acid adsorbed onto the surface of metal beads 60. One such type of bead is GeneCatcher™ Magnetic Beads (commercially available from Invitrogen, Carlsbad, Calif.).

Thus, the embodiment shown in FIG. 23 may include a reaction vessel including one or more defined sections, with a particular reaction mixture or part of a reaction mixture (e.g., including one or more components of a reaction mixture—primers, etc.) in different sections of the vessel. For example, referring to FIG. 23, a reaction vessel (such as a cassette) may include a chamber including a solution having the first probe (i.e., the second panel of FIG. 23—labeled “Recognition of Probe 1”) and a section including a solution having amplification primers (see the third panel of FIG. 23—labeled “Amplification”). The reaction vessel (such as a cassette) as in the figure may also include a chamber including a solution having the second probe (i.e., the fourth panel of FIG. 23—labeled “Recognition of Probe 2”) and a section including a solution having amplification primers (see the fifth panel of FIG. 23—labeled “Amplification”). With any target nucleic acid associated with metallic beads, (such as by being adsorbed onto the surface thereof—see the left-most panel of FIG. 23), a magnetic field may then be moved along the cassette in order to move the metal bead and thus the nucleic acid target sequentially through the various solutions (see the magnet, representing magnetic field moving from panel to panel in FIG. 23). The general use of such a magnetic field to move metal beads with adsorbed nucleic acid through such cassettes is described in Bordelon et al., Development of a Low Resource RNA Extraction Cassette based on Surface Tension Valves, Applied Materials and Interfaces, 2011, 3, 2161-2168, which is incorporated by reference herein in its entirety.

As shown in FIG. 23, upon hybridization of probe sequences to the pathogen nucleic acid (second or fourth chamber), magnetic beads will be moved to a chamber having amplification buffer (third chamber or fifth chamber), which contains the first primer (DSPA primer), second primer, DNA polymerase, and dNTPs. Thus, in this approach, a sample (i.e., patient DNA) is immobilized to magnetic beads, processed and moved to and through DSPA recognition and amplification buffers. The first and second probe molecules (shown at the top of FIG. 23) contain pathogen complements 62 (blank segment in Probe 1 and lined segment in Probe 2) and a universal DSPA construct 64 (bubbled segment). If the sample contains pathogen 1, Probe 1 will hybridize to it (via Probe 1's pathogen complementary component) and will be transported to the adjacent solution containing the universal amplification buffer (e.g., the second chamber in FIG. 23). Thus, if Probe 1 binds and is carried into the amplification buffer, amplification will occur via the DSPA principles described previously, above.

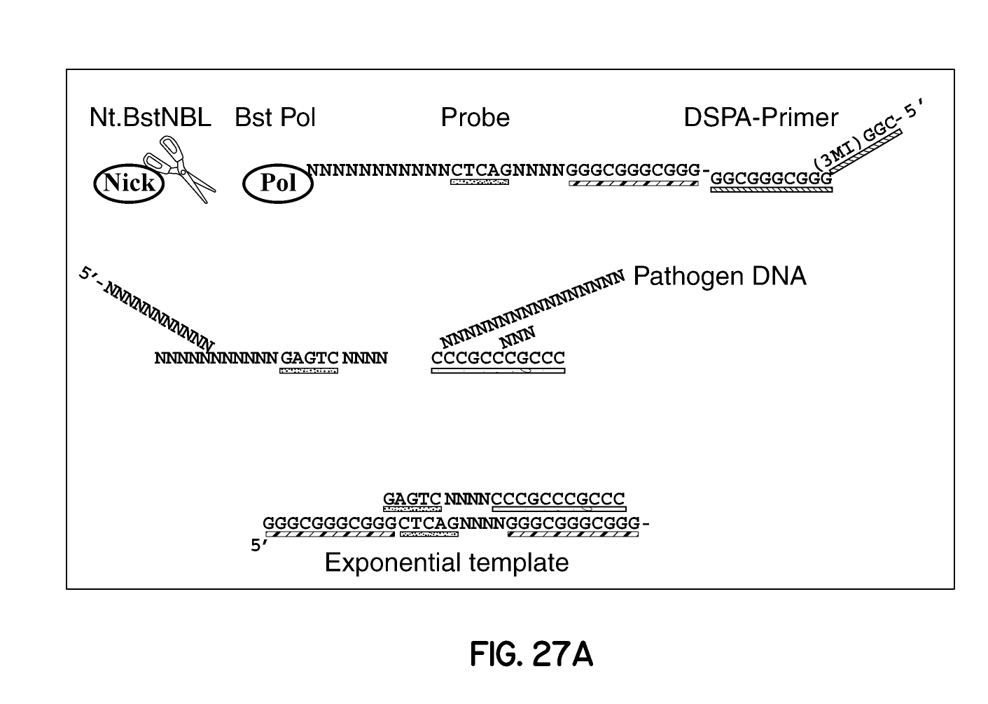

A schematic, which more specifically shows the details of the exponential DSPA that occurs in the amplification buffer, is shown in FIG. 24. Initially the DSPA primer 66 (dotted arrow) binds to the probe and replicates it. After spontaneous quadruplex 22 formation, the next DSPA priming/elongation occurs, which is accompanied by displacement of the first amplicon (shown at 68). The displaced strand contains a freely available primer-binding site 70 (bubbled and lined segment) for the second primer 72. The second primer binds and, after the primer binding, polymerase replicates the amplicon including the quadruplex at the 5′-end. After another step of amplification, a short duplex containing primer-binding sites for both primers is created and the reaction becomes exponential (right, FIG. 24).

Thus, once any such binding and amplification has occurred regarding Probe 1, the magnetic field is used to continue the progression of the metal beads/nucleic acid into the fourth chamber. If the sample then contains pathogen 2, Probe 2 will hybridize to it (via Probe 2's pathogen complementary component) and will be transported to the adjacent solution containing the universal amplification buffer (e.g., the fifth chamber in FIG. 23). Thus, if Probe 2 binds and is carried into the amplification buffer, amplification will occur via the DSPA principles described previously, above. Also, as described above, the amplification buffer can include a detectable signal (e.g., 2Ap incorporated into the dissociative sequence of the DSPA construct). And thus, one can determine which (if any) of the target nucleic acid segments are present in a sample by observing which chamber of a multi-well cassette produces the detectable signal.

As can be seen in the illustrated embodiment of FIG. 23, the sample DNA (black lines) hybridizes to Probe 1 molecules and not to Probe 2. Exponential DSPA thus occurs only in the second chamber. Thus, this approach allows for multiple diagnoses using a single signal (e.g., a fluorescence signal) and can be easily adapted to nucleic acid extraction cassettes.

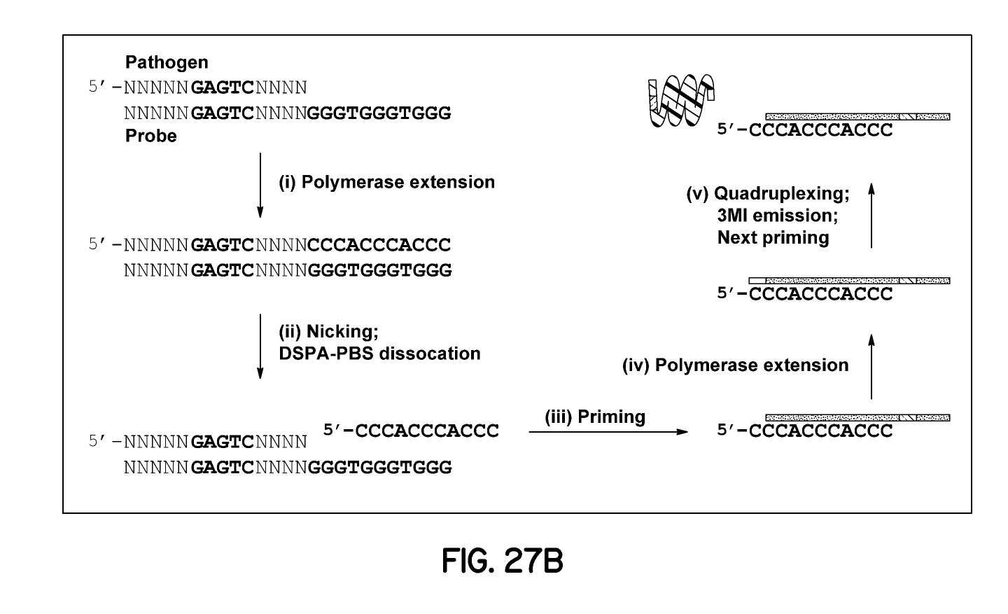

FIG. 25 demonstrates another embodiment of DSPA, which uses only one primer (as opposed to the two-primer approach used in the previous embodiment shown in FIGS. 23 and 24). This embodiment may also use a solid support—such as magnetic beads. This assay has less components than the previously described assay (as it has only one primer) and therefore it has a potential to be simpler. In the amplification process, the DSPA primer 66 (dotted arrow) binds to its PBS 74 (dotted segment). In this embodiment, the DSPA PBS includes a dissociative sequence/structure 22 at one end thereof. Upon replication, the dissociative structure (e.g., quadruplex) is unfolded (see 76 in FIG. 25). Next, quadruplexes are folded spontaneously at both sides (see 78 in FIG. 25), which releases the PBS and starts exponential DSPA with only one primer. Since there is only one primer versus two primers, the system is simplified, and this helps to inhibit background activity (e.g., inhibits any false signal before adding the construct with pathogen complement).

It is true that the DSPA PBS and dissociative structure segments 22, 74 shown in FIG. 25 may be complementary. Thus, it is possible that they will create stem-loops. In this case, PBS will be unaccessible for primers, which can inhibit DSPA. However, since the quadruplex is very stable, we hypothesize that one can find experimental conditions where quadruplex formation will be favored over stem-loop formation. For example, concentration of KCl can be increased (for instance, to 50 mM). This would favor quadruplex formation. However, the reaction also requires unfolding and replication of the quadruplexes by DNA polymerase. So, too much KCl would increase stability of the quadruplex and inhibit the unfolding process. Thus, one would find experimental conditions where quadruplex formation will be favored over stem-loop formation and the same time DNA polymerase will be able to unfold and replicate the quadruplex. This would be within the purview of one of ordinary skill in the art.

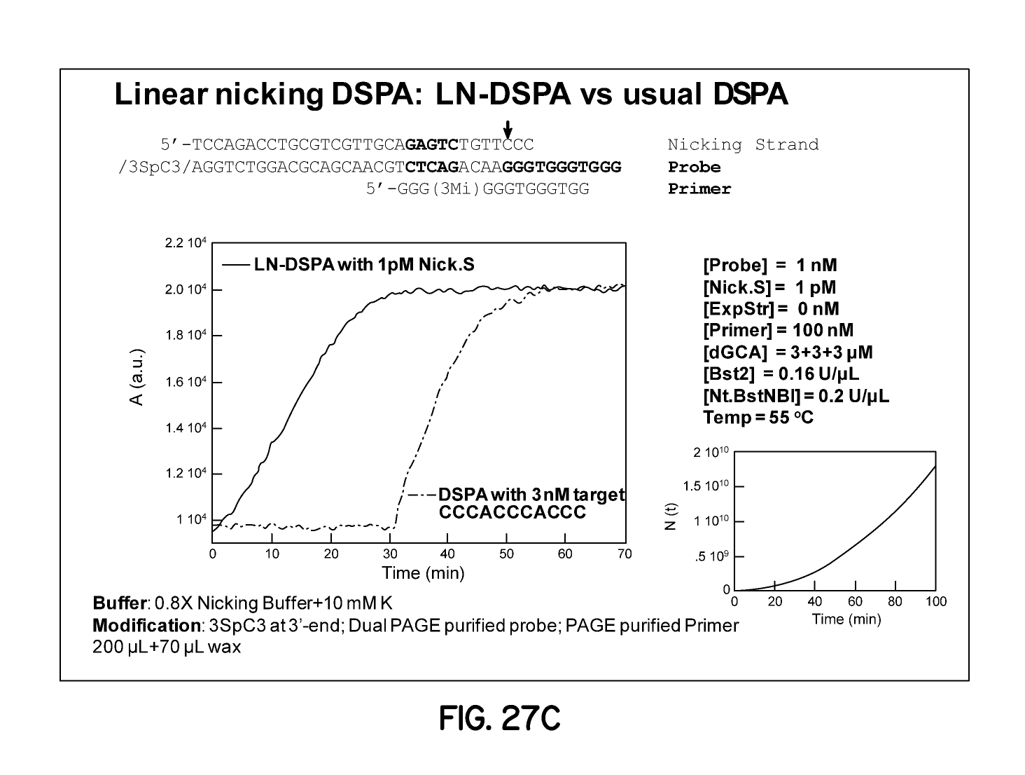

FIG. 26 demonstrates linear DSPA in another embodiment that uses a solid support, such as magnetic beads. The assay of this embodiment has been tested, as shown in Adams et al, Quadruplex priming amplification for the detection of mRNA from surrogate patient samples, The Royal Society of Chemistry, DOI: 10.1039/c3an02261g (2014), incorporated by reference herein in its entirety.

The embodiment shown in FIG. 26 is also shown as being used in a multi-chambered housing (and so the processes described above regarding the use of a magnetic field to sequentially move nucleic acid (e.g., adsorbed onto the surface of metal beads) through various chambers for binding and replication, apply to this embodiment as well. Thus, in this approach, a sample (i.e., patient DNA) is immobilized to magnetic beads, processed and moved to and through DSPA recognition and amplification buffers. A probe molecule 80 contains a pathogen complement 62 and a DSPA primer binding site 74. If the sample contains pathogen, the probe 80 will hybridize to it (via the probe's pathogen complementary component) and will be transported to the adjacent solution containing the amplification buffer (e.g., the second chamber in FIG. 26). Thus, if the probe binds and is carried into the amplification buffer, amplification will occur via DSPA principles.

To that end, in the illustrated embodiment of FIG. 26, a DSPA primer 66 is present in the amplification buffer, and is adapted to bind to the DSPA PBS 74. However, in the illustrated embodiment, this DSPA primer binds and replicates in the 3′ to 5′ direction. As such, as the primer elongates, it spontaneously conforms into its dissociative structure 22 (e.g., a quadruplex) and separates from the probe, thereby opening up the DSPA primer binding site 74 for binding of another DSPA primer 66. As a result, multiple dissociative structures will be formed (as seen at 82 in FIG. 26). And, the amplification buffer can include a detectable signal (e.g., 2Ap incorporated into the dissociative sequence of the DSPA construct). And thus, one can determine which (if any) of the target nucleic acid segments are present in a sample by observing which chamber of a multi-well cassette produces the detectable signal.

Those of ordinary skill in the art will recognize that the illustrated process of FIG. 26 will free from background activity typical for exponential assays. This version woks between 40° C. and 80° C., but requires sensitive optical methods. Such sensitive optical methods would be needed because this version is linear. Thus, bringing the signal to the sensitivity level of non-sensitive optics could take very long time (hours). However, since the signal formation is a monomolecular process, the very first signal can be detected early by sensitive optics. Note, other detection mechanism (beacons, Taqman™) are biomolecular and even with the sensitive optics would require amplification of the target molecules to have biomolecular interaction between target and probes.

Another embodiment uses principles of DSPA in combination with linear nicking amplification. As is known to those of ordinary skill in the art, nicking amplification is a method for in vitro DNA, which is isothermal, and replicates DNA at a constant temperature using a polymerase and nicking enzyme to exponentially amplify the DNA (generally at a temperature range of 55° C. to 59° C.). Thus, nicking amplification may be used as an alternative to PCR for amplification of nucleic acid. Thus, as the principles of DSPA can be used to improve amplification techniques (over that of standard PCR), those principles can also improve over the amplification techniques of nicking amplification.

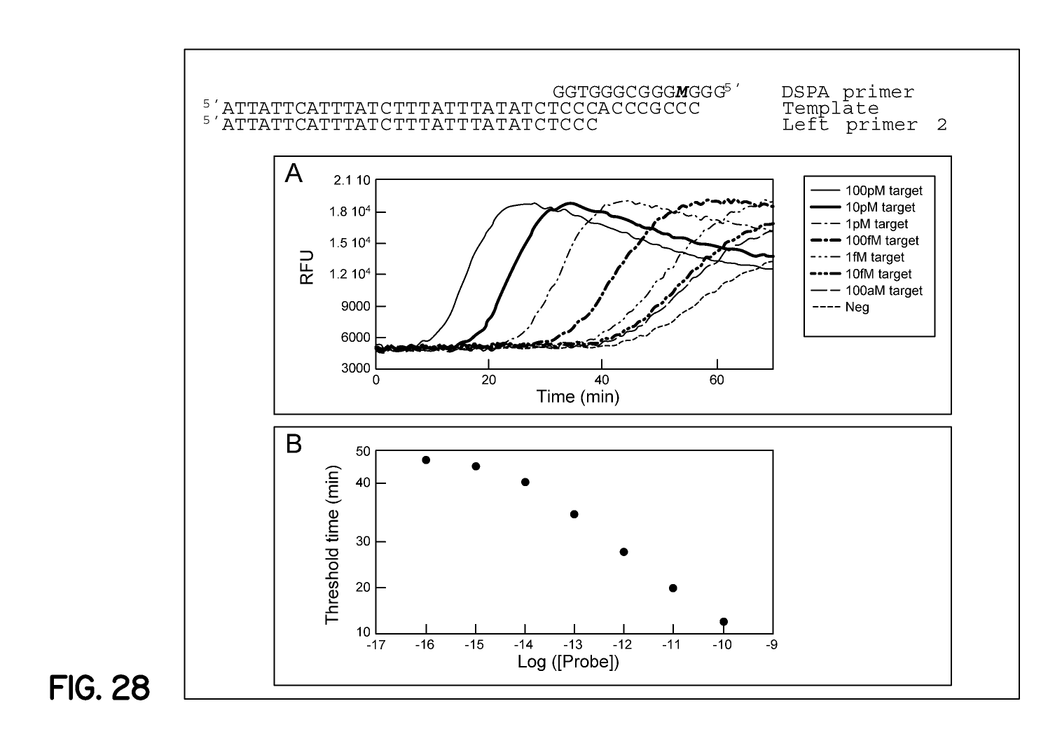

To that end, FIG. 27A demonstrates linear nicking DSPA (LN-DSPA), which is combination of two linear processes: linear DSPA and linear nicking amplification (LNA). LNA is based on the work of Van Ness et al., Isothermal reactions for the amplification of oligonucleotides, PNAS, Vol. 100, No. 8, (2003), pp. 4504-4509, incorporated by reference herein in its entirety, and uses the Nt.BstNBI nicking enzyme to recognize pathogen DNA after probe hybridization and initiate a linear process of DSPA-PBS (Primer Binding Site) formation. Thus, LNA allows for a linear process of DSPA-PBS formation, which is accompanied by signal amplification by DSPA. One advantage provided by the assay is that it does not include exponential amplification system and as a result avoids strong background activity.

Referring now to FIG. 27B, the formation of a DSPA-PBS by linear nicking amplification is more particularly shown. On the left side of the figure LNA is shown, and on the right side of the figure linear DSPA is shown. As can be seen in the upper left-hand portion of the figure, the probe has two specific segments: (1) the probe includes a CTCAG-5′ segment, which is used to create a binding site for the nicking enzyme [and because of this, the pathogen should have a complementary segment 5′-GAGTC (also shown in the figure]; and (2) the probe includes a GGGTGGGTGGG [SEQ. ID. NO. 12] segment at the 3′ end. The GGGTGGGTGGG [SEQ. ID. NO. 12] segment will be used to create a DSPA PBS segment having a CCCACCCACCC [SEQ. ID. NO. 13] sequence.

So, after probe binds to the pathogen and the first nicking event occurs, the construct shown in the upper left of FIG. 27B, will have been created. Next, as shown in step “(i)” of FIG. 27B, polymerase extension occurs, creating a complementary segment to the GGGTGGGTGGG [SEQ. ID. NO. 12] segment of the probe. Following this extension, another nicking event occurs [as shown in step “(ii)” of the figure], which causes the 5′-CCCACCCACCC [SEQ. ID. NO. 13] segment to be released/dissociated from the construct shown on the bottom left of the figure. Once this segment dissociates, it is then available to bind to DSPA primers. Such primers may be present in the solution already and initiate linear DSPA (as shown in steps “(iii),” “(iv),” and “(v)” of the right panel of FIG. 27B). Thus, as dissociative structures (e.g., quadruplexes) are repetitively formed during this process, a signal can be detected (such as 3MI, as shown in FIG. 27B), which indicated the presence of target DNA in the sample.

FIG. 27C compares LN-DSPA and linear DSPA. The reaction conditions used in the experiment are shown on FIG. 27C. As can be seen from the graph, in the presence of 1 μM nicking strand (equivalent to pathogen) LN-DSPA reveals a slope (black line), which is similar to the one obtained with linear DSPA (dashed line) in the presence of 3,000 higher nicking strand concentration (3 nM). This demonstrates almost 3,000 higher sensitivity in the case of LN-DSPA. Thus, by combining linear DSPA with the linear nicking assay (LNA), sensitivity increased dramatically.

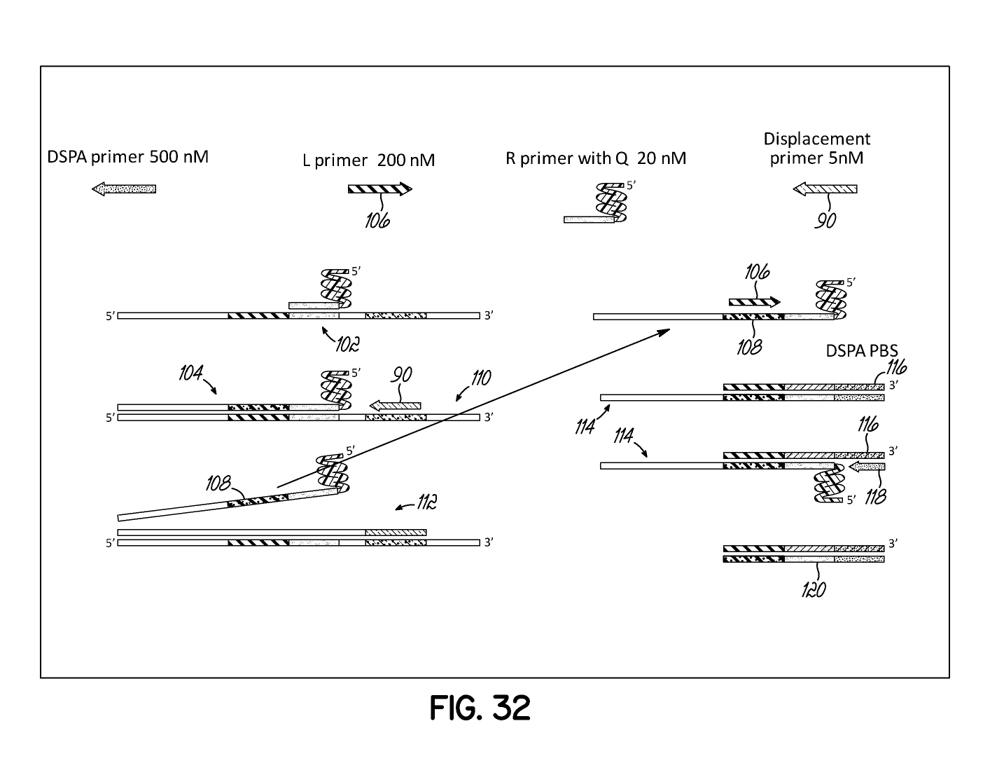

Apart from multi-well diagnostics described above, the DSPA principles described herein can be used/performed in a single housing (e.g., tube, cassette, etc.). One illustrative embodiment of this aspect of the present invention is shown in FIG. 31. The single-tube approach described here (and shown in FIG. 31) does not require any transporting mechanism. Initially, a right primer 84 with a quadruplex (or other DSPA) attachment 86 (lined segment) binds to a target nucleic acid sequence (e.g., pathogen), and initiates its replication (as shown at 88 in FIG. 31). Next, a displacement primer 90 binds near the right primer and initiates displacement of the first amplicon (as shown at 92 in FIG. 31). The displaced strand contains a primer binding site for left primer 94. This primer binds and replicates the amplicon (as shown at 96). Following this amplification, a dissociative structure forms, and frees a DSPA-PBS for binding of a DSPA primer (see 98). After another step of amplification, a short duplex 100 containing primer binding sites for both primers (non-DSPA primer and DSPA primer) is created and the reaction becomes exponential similar to the reaction described above with respect to FIGS. 23 and 24.

As can be seen, FIG. 31 shows isothermal DSPA using four primers. In this embodiment, the DSPA primer is used at a high nanomolar concentration, whereas the three other primers are used only during the initial cycle of amplification and therefore can be used at low nanomolar concentration. In addition, the assay may also work without the displacement primer 90 since it is only needed to displace the very first amplicon, which forms an unstable bimolecular duplex due to very low concentration. In such an alternate embodiment, one heating step can be introduced instead of the displacement primer.

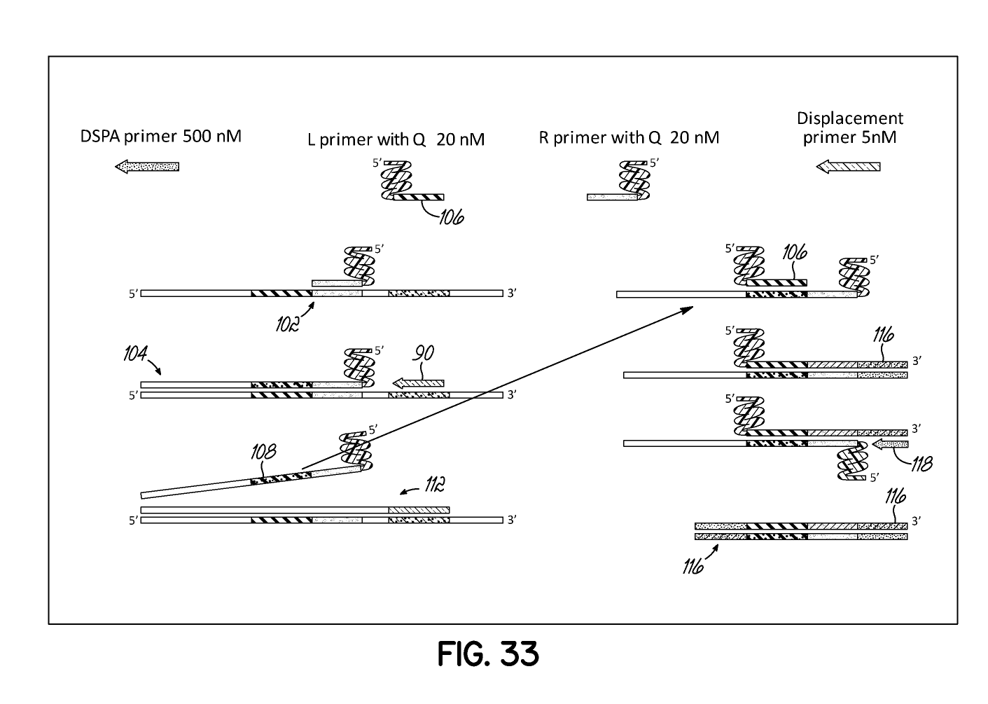

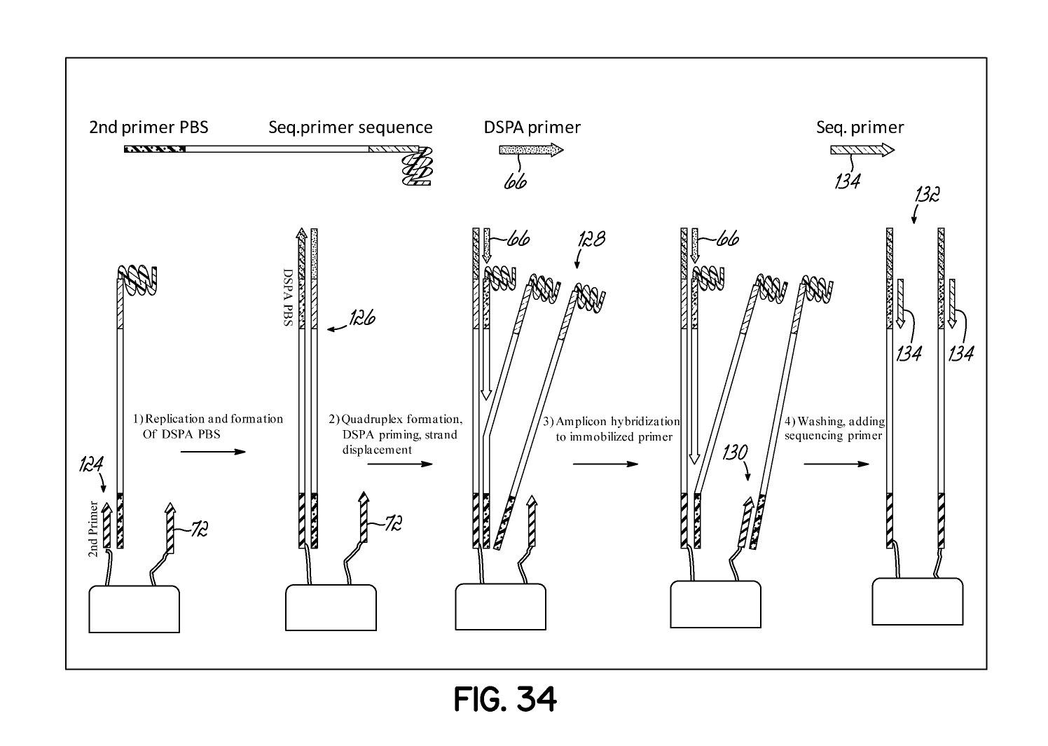

FIGS. 32 and 33 demonstrate yet other DSPA embodiments that do not require a solid support (e.g., magnetic bead) and can be used in a single chamber. FIG. 32 demonstrates a DSPA embodiment similar to that shown in FIG. 24 and FIG. 31. It requires 4 primers, [or alternatively, 3 primers with one temperature step (heating)—as described above with FIG. 31]. Two of the primers are used at higher concentrations and other two primers are used at lower concentrations (see FIG. 32). This is because the “R primer with Q” and “displacement primer” are used only at the initial stage of the amplification. After making the last construct, shown on FIG. 32, only first primers (DSPA and L) with the high concentrations are needed. In this embodiment, the right primer with the Q-attachment is not fluorescent, since it contains A instead of 2Ap. As used herein, a “Q-attachment” may be a formed quadruplex (or other dissociative structure), or a primer with a GGGTGGGTGGGTGGG [SEQ. ID. NO. 14] attachment (or other dissociative sequence).

The DSPA primer, which primes after production of DSPA-PBS, has 2Ap and can (i) give signal, and (ii) create PBS for exponential DSPA (Taq replicates 2Ap. As can be seen in FIG. 32, initially the right primer (R primer with quadruplex) binds to the pathogen 102 and the polymerase replicates the pathogen (see 104). The replicated strand in this embodiment already has a PBS for the L primer 106. However, it is not available as it is already bound to the pathogen strand. To make the PBS 108 available, a displacement primer 90 binds (see 110) and polymerase replicates the pathogen again. This is accompanied by displacement of the previously replicated strand (see 112). At this point PBS for the L primer 106 is available for priming (see right side of FIG. 32). Next, L primer 106 primes and upon replication quadruplex is unfolded (see 114) and a PBS 116 for DSPA primer 118 is created. At this point system becomes similar to the two-primer DSPA described above (with respect to FIG. 23), and amplification proceeds in a similar manner as described previously. The primary difference between the present embodiment and that previously described embodiment is that the present embodiment (as shown in FIG. 32) has a segment 120 between DSPA primer and L primer initially used for PBS for R primer.

FIG. 33 shows yet another embodiment that is based on the principles of the single-primer embodiment shown in FIG. 25—it uses only one primer at a higher concentration (see FIG. 33). This embodiment is similar to the embodiment shown above with respect to FIG. 32, with the only difference being that that both L primer and R primers have quadruplex attachments, which allow amplification of signal using only one primer (the DSPA primer) at the last stage of the process.

Initially, right primer (R primer with quadruplex) binds to the pathogen 102 and the polymerase replicates the pathogen. The replicated strand already has PBS 108 for the L primer 106, however it is not available. To make the PBS available, displacement primer 90 binds and polymerase replicates the pathogen again (see 104). This is accompanied by displacement of the previously replicated strand (see 112). At this point PBS 108 for the L primer 106 is available for priming (as shown on the right side of FIG. 33). Next, L primer with the quadruplex attachment binds and, upon replication, the quadruplex is unfolded and a PBS 116 for a DSPA primer 118 is created (see 122). The last construct shown on the lower right side of FIG. 33 thus has DSPA PBS 116 at both ends and therefore does not require a second primer as shown in the embodiment of FIG. 32. Thus, difference between the embodiment of FIG. 32 and the embodiment of FIG. 33 is that the latter uses only one (DSPA) primer in signal amplification, while former requires an additional primer.

Principles of DSPA are also useful in preparation of DNA libraries and clone generation for next generation sequencing methods. As described above in the Background section, with techniques for library preparation, the random attachment of two different adapters (A and B) to DNA fragments, employed in all NGS, is a very inefficient way of library preparation. First, due to producing homo-adapter fragments (A-A and B-B instead of A-B)—as described above—a significant amount of DNA fragments is wasted, which necessitates a large amount of genomic DNA and multiple sequencing reactions. Second, the library enrichment process (e.g., cleaning from homo-adapter fragments to avoid further complications and expenses) significantly elongates workflow. Third, enriched DNA fragments can be sequenced only in one direction (complementary strands are discarded).

However, DSPA, is able to conduct isothermal generation of DNA clones using a single primer, and thus has a potential to revolutionize the sequencing process by (i) skipping the enrichment step; (ii) using very little genomic material (ideally, whole genome can be sequenced in both directions using a single copy); and (iii) making pair-end sequencing reaction integral part of any sequencing. This will be described in greater detail below.

Further, as described above in the Background section, with regard to clone generation in NGS, emPCR and bPCR are difficult reactions requiring thermo-cycling and solution-cycling, respectively. In addition, due to monomolecular nature of interactions at solid support, product self-annealing dominates over priming, which severely decreases PCR efficiency. And so, recently an isothermal method, called “Wildfire,” was developed (5500×1 Wildfire, Life Technologies), which takes advantage of the monomolecular nature of priming process of immobilized primers. The wildfire clone generation is simpler than emPCR or bPCR. However, it requires an initial temperature step for library hybridization to solid phase primers and relies on unspecific unfolding of DNA ends. In addition, strand-displacement priming from the free end of the DNA is undesired, since this can result in diffusion of amplicons, which could initiate new clone formation somewhere else. Thus, wildfire approach relies on two different priming processes. In first, immobilized primers are able to displace previous (already extended) primers isothermally and initiate amplification. In second priming events, primers should not have this primer-displacement ability and prime only after PBS is released.

However, since DSPA self-dissociative primers can be selectively attached to the DNA ends, it can significantly improve Wildfire clone generation. In addition, as mentioned above, DSPA can further revolutionize clone amplification by using mono-adapters during library preparation.

Illustrative embodiments of such methods will now be described:

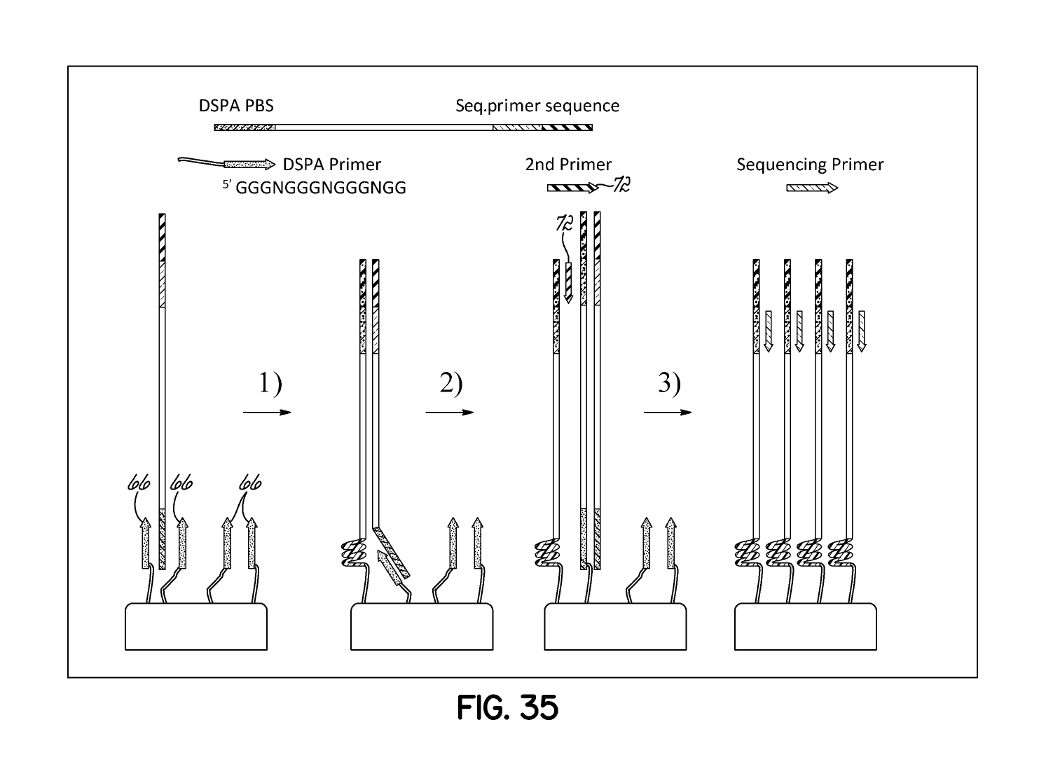

A first such embodiment includes the use of a DSPA primer and a non-DSPA primer in conjunction with a solid support in sequencing. Referring now to FIG. 34, this assay provides for isothermal amplification of DNA for sequencing and allows that a DSPA primer 66 and a second primer 72 (being a non-DSPA primer) are attached to the DNA during adaptor ligation. The assay of this embodiment is similar to the “two-primer DSPA using magnetic bead” approach as shown in FIGS. 23 and 24. The primary difference in the present embodiment is that second primer is immobilized and the DSPA primer is free in solution (see FIG. 34). As a result, a template can dissociate from the surface similarly to emulsion PCR.

Referring to FIG. 34, initially DNA hybridizes to the second primer, which is attached to the solid support (beads or flow cells)—see 124 in FIG. 34. Polymerase replicates the DNA including the dissociative sequence (e.g., quadruplex) at the 5′-end—see 126. After spontaneous quadruplex formation, the next cycles of DSPA priming/elongation occur, which are accompanied by displacement of the previous amplicons—see 128. The displaced strands, which contain primer-binding sites for immobilized primers, are free in solution and are ready to bind to other immobilized primers—see 130. Upon displacement, amplicons are released to solution; however, since this occurs in close vicinity to the immobilized primers, it is hypothesized that they can bind to the immobilized second primers without diffusion from the solid support. Thus, in addition to emulsion-based clonal amplification, the assay described here can be used for cluster generation on flow cells. After a washing step, constructs are ready for sequencing—as shown at 132, using sequencing primer 134.

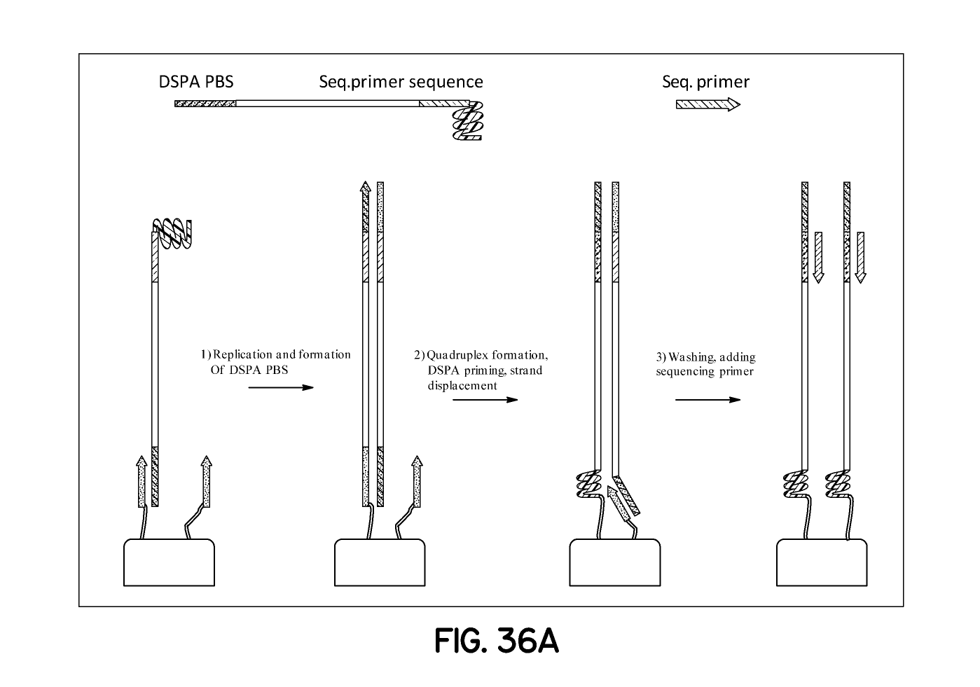

In the above embodiment of FIG. 34, the second primer is attached to the support while the DSPA primer is free in solution. In an alternate embodiment, and referring now to FIG. 35, the DSPA primer 66 is immobilized while the second primer 72 is free in solution. Here, the amplification is similar to Wildfire clone generation used in 5500×1 W (Life Technologies). A DNA library is prepared by attaching two adapters to DNA fragments (one containing DSPA primer and other containing second primer and sequencing primer). Thus, DSPA primers (66, FIG. 35) are immobilized and second primer (72) is free in solution. Upon hybridization to immobilized DSPA primers the DNA fragment is replicated, and upon extension, a dissociative sequence (e.g., quadruplex) forms and allows the next round of priming/replication, which is accompanied by displacement of first amplicon (see step 1). Next, the second primer 72 hybridizes to its PBS at the free end of the amplicon and replicates it (see step 2). At this point process becomes exponential. The DSPA-based assay has advantages over simple Wildfire clone generation in that: (i) the amplification does not require first temperature step; (ii) the amplification is driven by the energy of dissociative structure (e.g., quadruplex) formation; and (iii) the second primer cannot prime spontaneously and initiate replication, which prevents the amplicons from diffusion and starting new clones somewhere else.

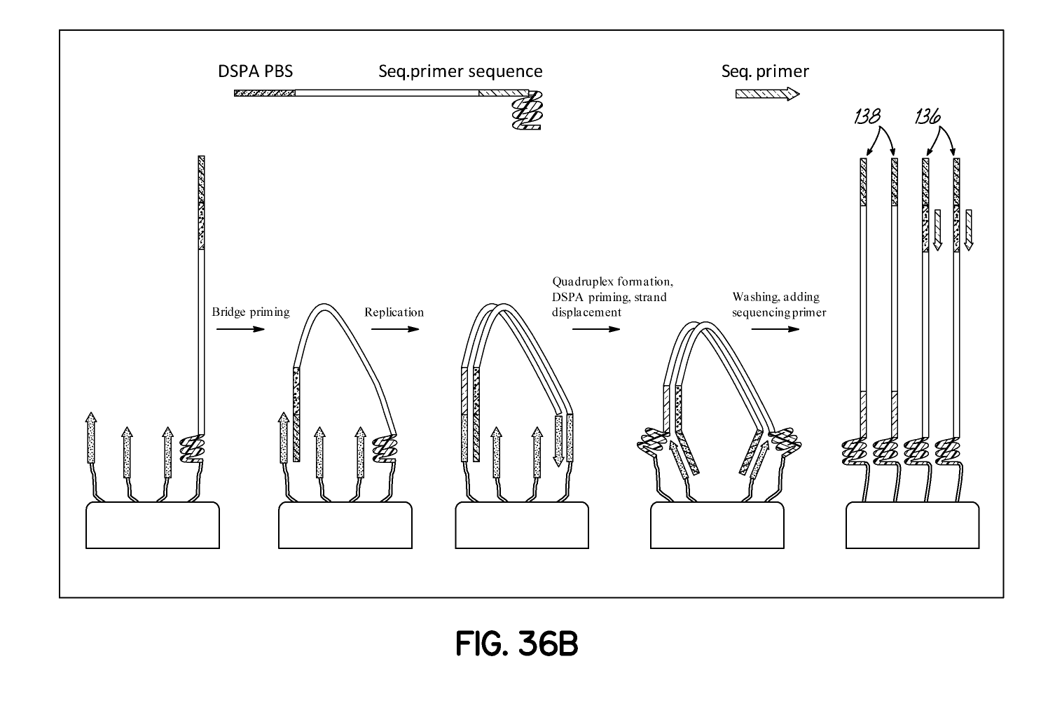

Another embodiment involves a single primer assay (i.e., an assay using only a DSPA primer in conjunction with a solid support)—as opposed to the two-primer embodiments described above. More specifically, this embodiment is an assay that uses only DSPA primer attached to the solid support and is suitable for cluster generation on flow cells since amplicons are permanently attached to the solid support (FIGS. 36A and 36B). The DNA has DSPA primer binding site at the 3′-end and quadruplex, or its shorter version, at the 5′-end and does not require any additional primer. The amplification can run in two different pathways. First, DNA binds to the primer, and after replication and spontaneous quadruplex formation DNA strand is ready to bind to another primer (FIG. 36A). This process, which is called “parallel” amplification, is a linear process. The second pathway can run through the bridge formation (FIG. 36B), which is exponential process. As a result, around half of the amplicons 136 will be ready for sequencing, white another half 138 can be used for paired end sequencing assuming that an appropriate primer will be attached during adaptor ligation.

Yet another embodiment involves the use of DSPA principles for mono-adapter DNA clone generation. In this assay, a DNA library is made with a mono-adapter. The shortest DSPA mono-adapter is a 15-bp GGGNGGGNGGGNGGG [SEQ. ID. NO. 11] (G3N) duplex. “N,” as used here can independently represent any nucleotide or alternate base, (thus, each “N” can be the same nucleotide as the other “N”s, or different. If needed, the sequence can be elongated at both ends. Additionally, the adapter ligation should be performed in the absence of K+ ions (to avoid adapter dissociation), which will be added before unfolding the DNA library (if needed, ligation can be performed in the presence of K+ ions, but adapters should be performed in the absence of K+ ions).

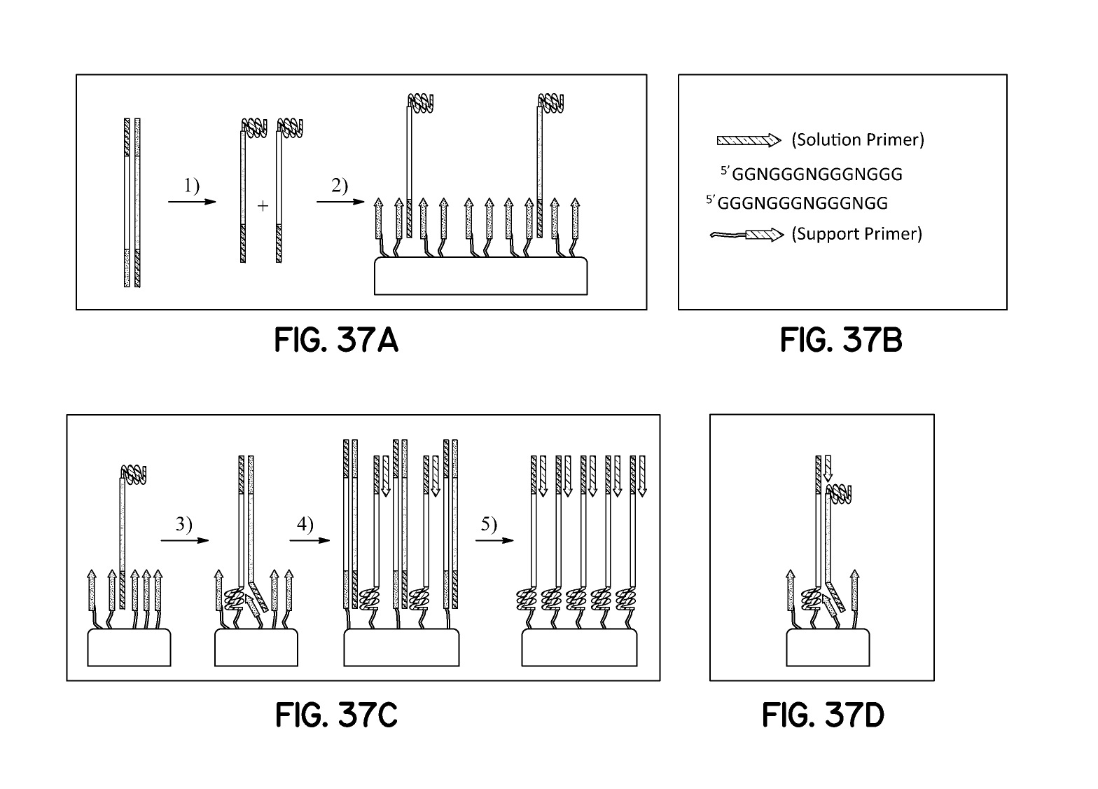

Unfolded DNA with quadruplex at this 5′-end is not able to form a stem-loop structure (see step 1, FIG. 37A) and both strands hybridize to immobilized primers (see step 2, FIG. 37A). Next, an amplification solution, containing all components including solution or free primer (FIG. 37B), will be pumped into the flowcell. Immobilized primer and solution primer are almost identical having same parent sequence, G3N, but with one principal difference: the immobilized primer is missing one G at the 3′-end and solution primer is missing one G at 5′-end. Thus, the immobilized primer forms a quadruplex (or other dissociative structure) upon adding missing G by polymerase, self-dissociates and allows next priming isothermally. However, the free primer is not able to form a quadruplex (or other dissociative structure) upon polymerase extension and acts as a normal primer.

After replication, dissociative structure (e.g., quadruplex) formation, and the next cycle of priming (see step 3, FIG. 37C), strand-displacement replication releases initial amplicon, which is primed from the free end by solution primer (see step 4, FIG. 37C). At this point amplification becomes exponential. At the end (see step 5, FIG. 37C), mobile amplicons are washed and sequencing solutions introduced. In this assay, for amplification and sequencing the same primers are used. If needed, one can elongate sequencing primer by elongating the adapter.

After first round of replication, the DNA library strand can start the priming process from the free end since it contains a full-length quadruplex (FIG. 37D). This can result in diffusion of the library strand and starting the second clone somewhere else, which is not necessarily negative fact since this random diffusion can be happen only to initial library strand (all amplicons are missing terminal guanine at 5′-end which inhibits quadruplex formation). If needed, there is two ways to avoid new clone formation: (i) after first replication step remove the original strand by a washing step; (ii) to find experimental conditions unfavorable for quadruplex formation at free end, because at solid supports DSPA priming has an advantage being monomolecular.

The various aspects of the present invention will be described in greater detail with respect to the following nonlimiting Examples.

### Example 1

This Example describes development of primers for use in an isothermal amplification process. As described above, the primers used in various embodiments of such a process may be of a sequence that does not initially form a dissociative structure (such as a quadruplex), but that will do so upon extension of the sequence during amplification. One exemplary embodiment of such a primer sequence is G3T-ss13.

Role of Cations and Terminal Guanines in Quadruplex Formation.

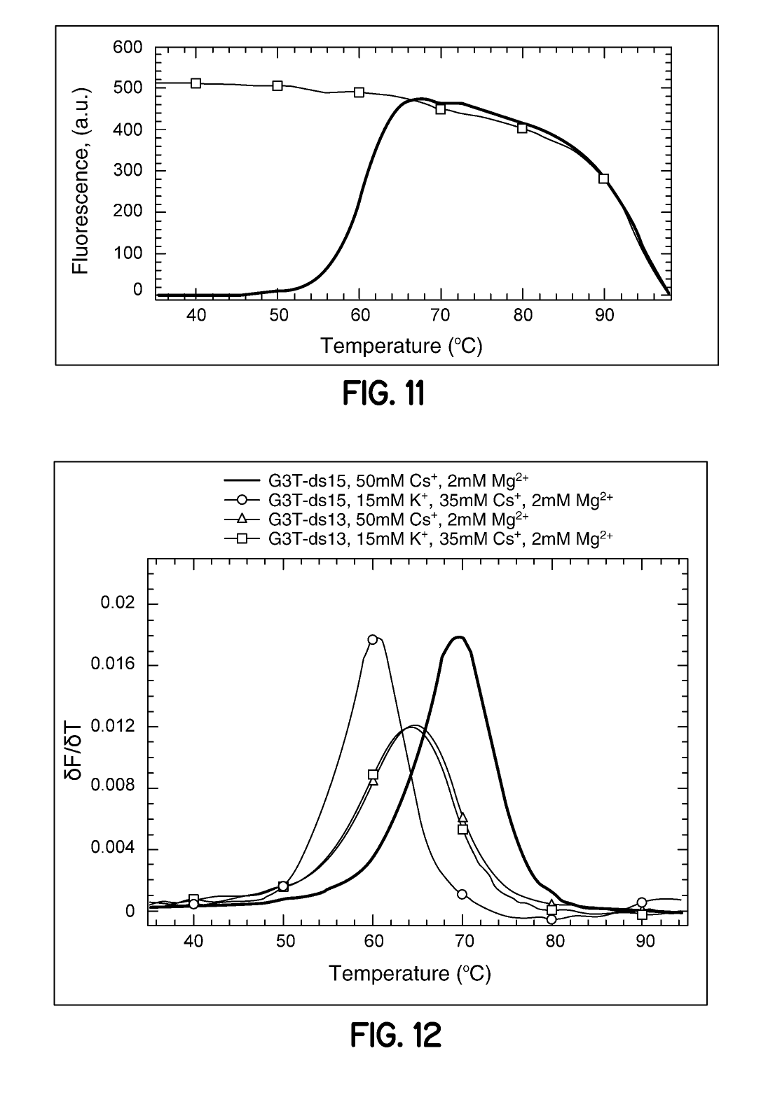

FIG. 9 demonstrates fluorescence unfolding experiments of G3T-ss15, G3T-ss14, and G35-ss13. Unfolding of G3T-ss15 was performed in the presence of 50 mM monovalent cations, Na⁺ (-∘-), K⁺ (black line) and Cs⁺ (--). In the case of Na⁺ ions the melting curve reveals the sigmoidal behavior characteristic of monophasic transition with Tₘ ˜45° C. The transition corresponds to unfolding of the quadruplex, which is accompanied by quenching of 2Ap fluorescence by adjacent guanines in the unfolded quadruplex. As expected [as shown by Jing, N., Rando, R. F., Pommier, Y. and Hogan, M. E. (1997) Ion selective folding of loop domains in a potent anti-HIV oligonucleotide, Biochemistry, 36, 12498-12505], the potassium salt of G3T-ss15 is very stable with a Tₘ of ˜88° C. (black). Thus, both Na⁺ and K⁺ ions are able to fold quadruplexes, however the latter is almost 45° C. more stable. In the presence of Cs⁺ ions G3T-ss15 does not reveal any measurable fluorescence over the entire temperature range, which suggests that Cs⁺ does not support quadruplex formation [Kankia, B. I. and Marky, L. A. (2001) Folding of the thrombin aptamer into a G-quadruplex with Sr(2+): stability, heat, and hydration, Journal of the American Chemical Society, 123, 10799-10804]. The results are in agreement with observations that K⁺ ions with ionic radii of 1.33 Å are the optimum size for a cation to enter the inner core of G-quartets, while Cs⁺ ions with ionic radii of 1.69 Å are too big [Jing, N., Rando, R. F., Pommier, Y. and Hogan, M. E. (1997) Ion selective folding of loop domains in a potent anti-HIV oligonucleotide, Biochemistry, 36, 12498-12505].

The role of terminal guanines in quadruplex formation in the presence of K⁺ ions was studied similarly. Deletion of a single guanine at the 3′-end, G3T-ss14, significantly destabilized the quadruplex (FIG. 9, -Δ-). However, it is still able to create some structure at lower temperatures. Deletion of another guanine, G3T-ss13, almost completely inhibits quadruplex formation (-□-). Thus, the experiments shown in FIG. 9 suggest that (i) in the presence of Cs⁺ ions mixing of full-length G3T-ss15 to its complementary sequence should result in a DNA duplex; and (ii) in the presence of K⁺ ions the truncated variant, G3T-ss13, should also be able to form a duplex.

Role of Cations and Terminal Guanines in Duplex Formation.

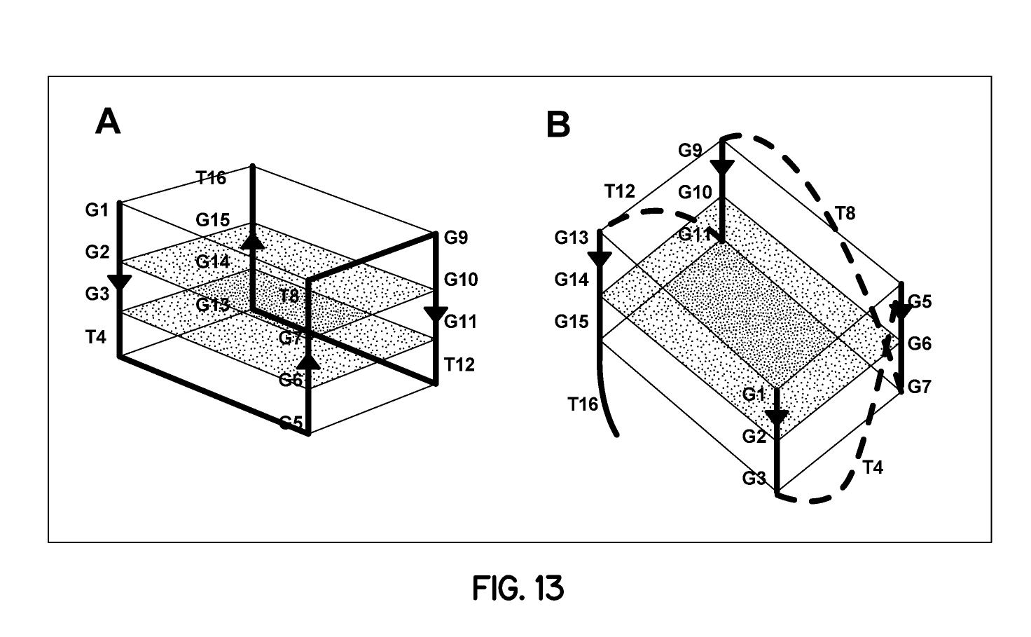

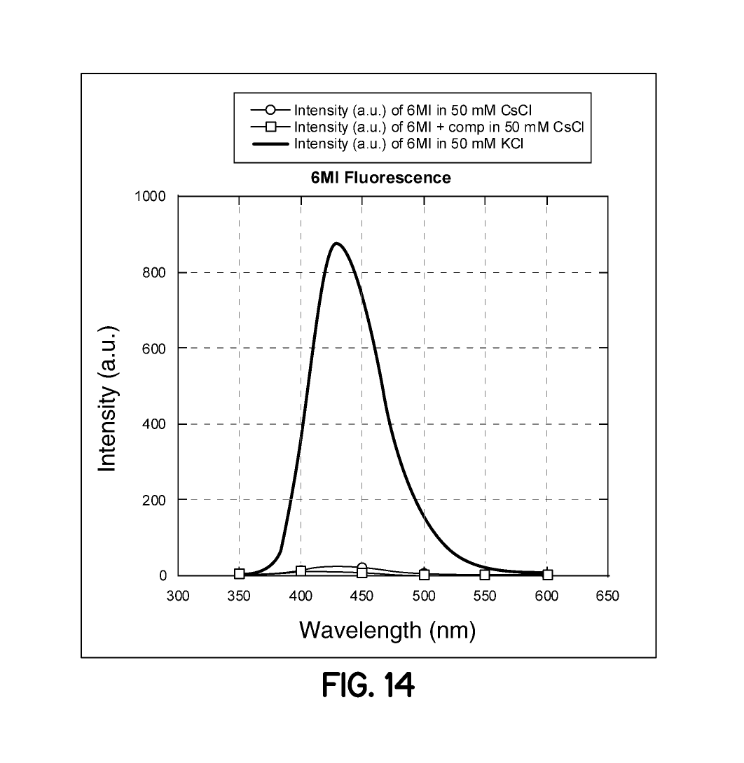

To mimic DNA conformational changes that take place upon the amplification reaction, the fluorescence melting of the G3T-ds15 duplex was studied in amplification buffer (15 mM KCl, 35 mM CsCl, 2 mM MgCl₂, 10 mM Tris-HCl, pH 8.7) (FIG. 10). To ensure that G3T-ds15 initially anneals to its complementary strand (as double-helix), the sequences were annealed in the presence of CsCl followed by later KCl addition. (K⁺ is a quadruplex forming cation, while Cs⁺ does not support quadruplexes [as described in Kankia, B. I. et al. (2001) Folding of the thrombin aptamer into a G-quadruplex with Sr(2+): stability, heat, and hydration, J Am Chem Soc, 123, 10799-10804, incorporated by reference herein in its entirety.]). The duplex is formed by annealing a shorter version of 2Ap-G3T (unable to form a quadruplex), such as G3T-ss13, to the target sequence with subsequent addition of the missing bases by Taq polymerization. The heating curve (black curve, FIGS. 10 and 11) reveals two separate transitions with midpoints at 60° C. and ˜95° C. The transition at 60° C. corresponds to duplex unfolding, which is accompanied by an increase in fluorescence due to quadruplex formation of released G3T-ss15. The second transition at ˜95° C. corresponds to the melting of the quadruplex accompanied by fluorescence quenching of 2Ap due to stacking interactions of adjacent guanines in unstructured 2Ap-G3T. The second transition is completely reversible during the cooling process (-□- in FIGS. 10 and 11). However, no duplex refolding was observed, which clearly indicates that the quadruplex stays folded at lower temperatures in the presence of the complementary strand. In separate isothermal experiments at 40° C., the complementary strand was added to a preformed G3T-ss15 quadruplex, which didn't affect the fluorescence spectrum of the quadruplex (data not shown). Thus, both melting and isothermal mixing experiments show that the quadruplex is very stable and the complementary strand is unable to invade the structure.

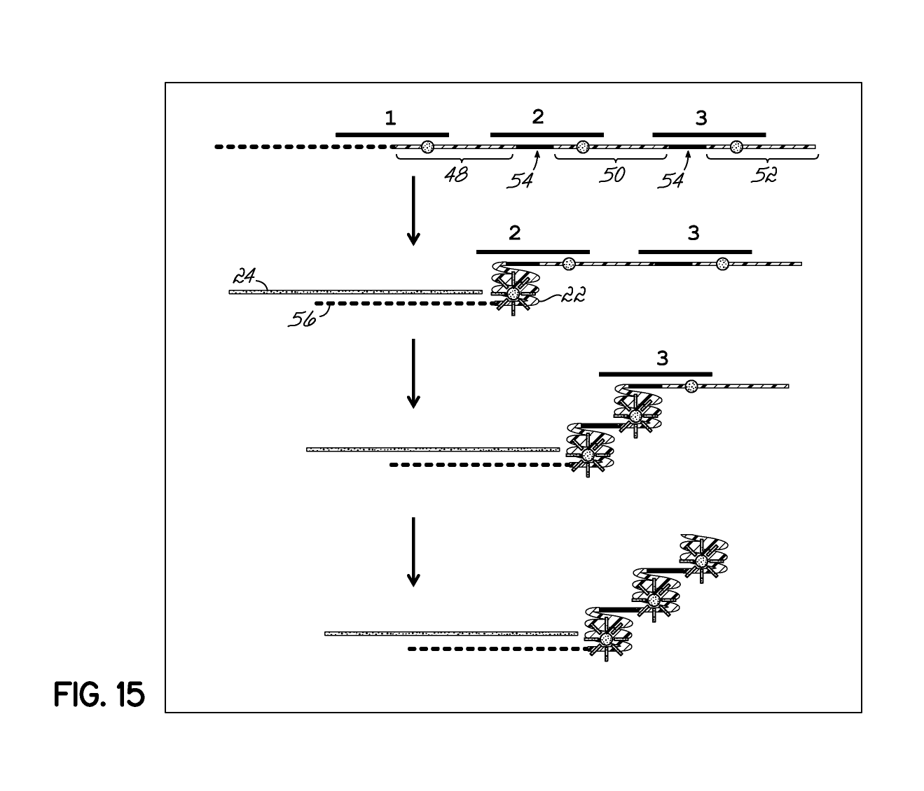

It is noted that the duplex melting temperature (˜60° C.) measured in the presence of the quadruplex forming cation KCl (FIGS. 10 and 11) is significantly lower than the Tₘ=70° C. of the same duplex measured under experimental conditions unfavorable for quadruplex formation (50 mM CsCl and 2 mM MgCl₂), or predicted from nearest-neighbor analysis of equilibrium unfolding [Zuker, M. (2003) Mfold web server for nucleic acid folding and hybridization prediction, Nucleic acids research, 31, 3406-3415]. To compare thermal stabilities of the G3T-ds15 duplex in the presence and absence of K+, UV absorption was employed (FIG. 12). In the presence of K+ ions, G3T-ds15 unfolds at 60° C. (-∘-), which is in excellent agreement with results of the fluorescence measurements shown in FIG. 10. In the absence of K+ ions, the duplex is significantly more stable and unfolds at 70° C. as predicted from nearest-neighbor analysis of equilibrium unfolding [Zuker, M. (2003) Mfold web server for nucleic acid folding and hybridization prediction, Nucleic acids research, 31, 3406-3415]. Note an additional small peak at 93° C. in the presence of K⁺, which corresponds to quadruplex unfolding and again agrees with fluorescence measurements shown in FIG. 10. Additional melting experiments of the G3T-ds15 duplex in the presence of K⁺ performed at slower heating rates (0.5° C./min and 0.1° C./min) further shifted the transition to lower temperatures (data not shown). Thus, in the presence of K⁺, unfolding of the duplex is a non-equilibrium process due to quadruplex formation of the released strands, which significantly destabilizes the duplex. FIG. 12 also demonstrates unfolding of G3T-ds13 in the presence and absence of K⁺ ions. Since G3T-ss13 is not able to form a quadruplex (see FIG. 9), G3T-ds13 duplex melting profiles are identical in the presence and absence of K⁺ ions with Tₘ=65° C. As expected, in the presence of Cs⁺ ions the longer duplex, G3T-ds15, is more stable than the shorter duplex, G3T-ds13. However, in the presence of K⁺ the opposite is true: the shorter duplex is ˜5° C. more stable than the longer one. This result illustrates the potential for isothermal amplification; at appropriate temperatures, the primer is more stable before elongation, which facilitates primer dissociation and the next priming round without the need for thermal denaturation.

As a result, a dissociative conformation is assumed and signal evolves after adding two guanines to a primer, such as G3T-ss13. Due to the fact that high amounts of dNTP (˜0.5 mM) may inhibit Taq, the reaction shown in FIG. 4, panel B, which only requires a two-guanine extension in the described embodiment, may allow very high concentrations of signal molecules to form, such that the signal may be detected by the unaided eye.

Further, as shown in FIG. 12, in the presence of K⁺ ions, the DNA duplex, G3T-ds13 is ˜5° C. more stable than full length sequence, G3T-ds15. Therefore, one might predict that the primer in FIG. 4 will displace the first sequence segment of the stem-loop and initiate polymerization in the absence of target nucleic acid. However, this is unlikely since the stem-loop is a monomolecular structure, which makes it entropically more favorable than the bimolecular complex formed by the primer and the stem-loop. For instance, a monomolecular 17-bp duplex is 20° C. more stable than the corresponding 15-bp bimolecular duplex. In addition, a minimum amount of K⁺ (for instance, 2 mM) may be used to further increase stem-loop stability, and increase total ionic strength in reaction buffers to avoid stem-loop unfolding by accidental temperature increase.

### Example 2

### Non-Enzymatic Amplification

In this prophetic example, the DNA stem-loop 5′GGGAGGGCGGGTGGG(T)₁₄GGCCCGCCCTC (underline=quadruplex forming sequence, bold=loop, italic=stem) [SEQ. ID. NO. 15] will be studied in the absence and the presence of target sequence, 5′CC(A)₁₄CCCA [SEQ. ID. NO. 16]. The estimated Tₘs and free energies are: 51° C. and −8 kcal/mol for the stem-loop and 72° C. and −15 kcal/mol for the bimolecular complex [Zuker, M. (2003) Mfold web server for nucleic acid folding and hybridization prediction. Nucleic acids research, 31, 3406-3415]. Thus, it is expected that the 20 bp target-loop complex should unfold the stem-loop structure. The 4-bp terminal segment of the dissociative-structure-forming sequence (underlined), TGGG, involved in complex formation is too short to inhibit quadruplex formation. Thus, upon stem unfolding, the quadruplex (or other dissociative structure) should form. A combination of UV and fluorescence melting experiments of stem-loop plus target will test these predictions. In the case of successful unfolding of a stem-loop and quadruplex formation, UV-unfolding should reveal only one peak at ˜50° C. Alternatively, two peaks may be observed: one for bimolecular complex melting at ˜50° C. and a second for the monomolecular stem-loop at ˜70° C. Any alternative two-peak observation may be the result of refolding of the stem-loop structure after melting of the complex, which means that the quadruplex was not folded.

Further Experimentation.

Stem-loops A and B may need to be altered to minimize the overlap between the quadruplex forming sequence and the target. In addition, loop and stem sequences also may need to be altered to shift Tₘs of the complex and the stem-loop. Next, the kinetics of target binding to and dissociation from the stem-loop will be investigated using the 2Ap fluorescence. Finally, complementary stem-loop will be designed for exponential increase of signal.

In such further experimentation, suitable DNA constructs will be designed by UV and fluorescence unfolding in the absence and presence of target molecules. Signal amplification will be monitored by fluorescence measurements of the most sensitive probe designed. Signal amplification will be monitored by the unaided eye using an appropriate excitation source.

### Example 3

### FRET-Based Probes

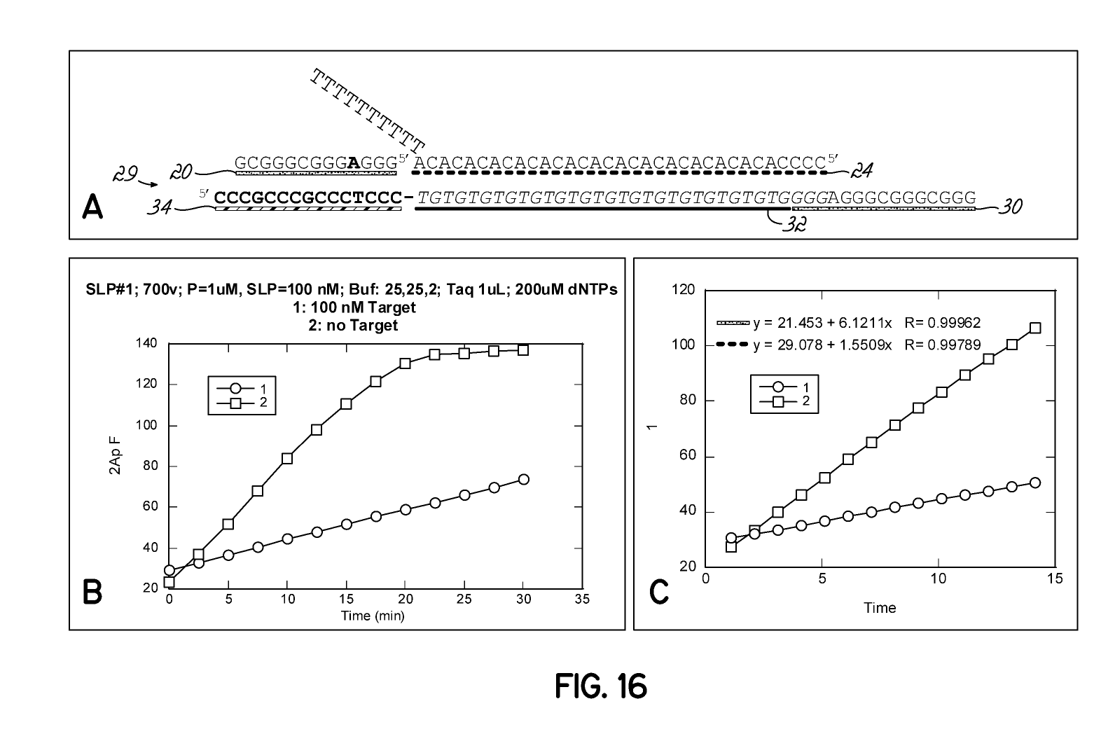

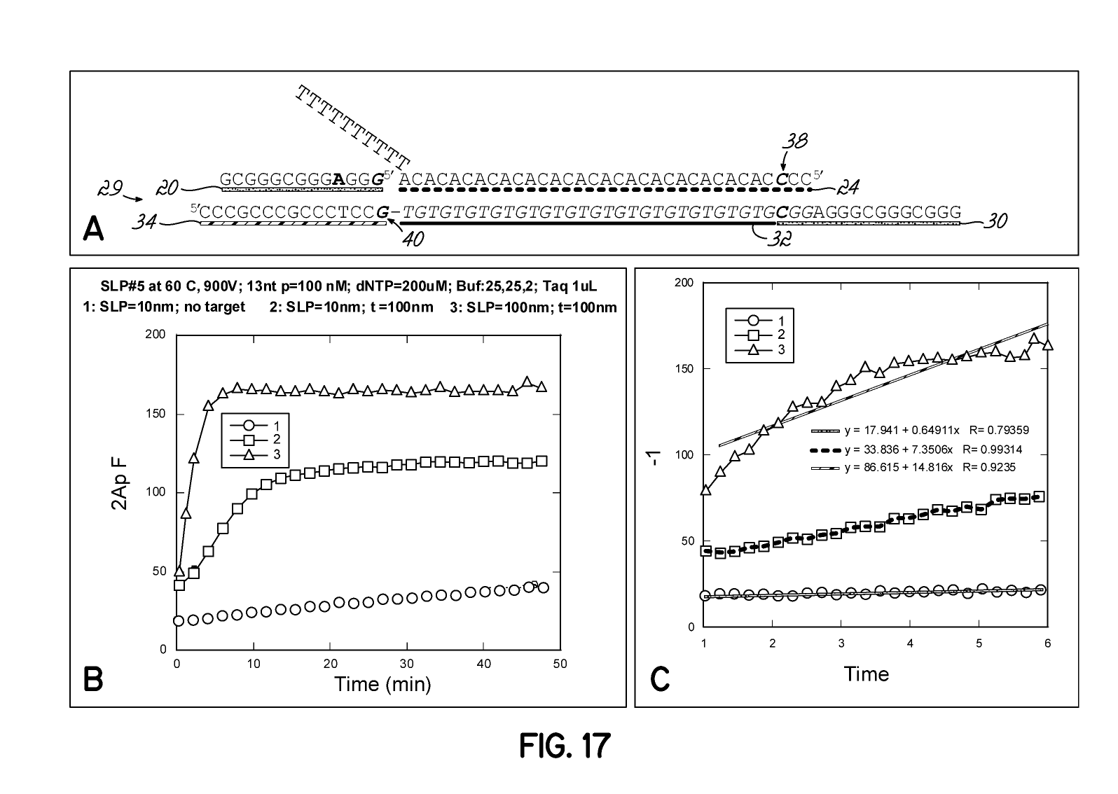

The hypothetical model of the parallel structure of a quadruplex shown in FIG. 13, panel B is based on thermodynamic and spectroscopic studies. Three G-quartets were assumed because of higher thermal stability of the G3T-ss15 quadruplex when compared with the quadruplexes with two G-quartets [Kankia, B. I. and Marky, L. A. (2001) Folding of the thrombin aptamer into a G-quadruplex with Sr(2+): stability, heat, and hydration. Journal of the American Chemical Society, 123, 10799-10804; Hardin, C. C., Perry, A. G. and White, K. (2000) Thermodynamic and kinetic characterization of the dissociation and assembly of quadruplex nucleic acids. Biopolymers, 56, 147-194]. However, two G-quartets in the G3T-ss15 sequence with three diagonal GT loops cannot be excluded. To test this possibility, substitution at positions 3, 7 and 11 will be made and studied for their effect on quadruplex formation. Depending on the outcome, incorporation of 2Ap in positions 3, 7 and 11 will also be tested.

The sensitivity of the probes will be estimated by fluorescence measurements before (quenched state) and after (emitted state) adding K⁺ ions and before (quenched) and after (emitted) adding missing guanines. Multiplexing capability will be tested by actual amplification of various segments of a plasmid DNA using four different primers with different fluorescence properties. Suitable primers for multiplexing will be designed using UV-melting experiments.

### Example 4

Due to its sensitivity, polymerase chain reaction (PCR) is a method of choice for diagnostics. However, as described above, PCR relies on thermal cycling, which is not compatible with the goals of point-of-care (POC) diagnostics. Principles of the present invention demonstrate that dissociative structures, such as a monomolecular DNA quadruplex, GGGTGGGTGGGTGGG [SEQ. ID. NO. 14] (G3T), can turn PCR into an isothermal method by using specific primers, which upon polymerase elongation, self-dissociate from the PBS. The G3T sequence, as used in this Example, is capable of forming a quadruplex structure with unusually high thermal stability. In contrast, the starting primer, which is a truncated version of G3T, is missing guanine residues critical for quadruplex formation. As a result the truncated sequence primes without complication. When the polymerase adds the missing guanines, the extended primer spontaneously folds into a DNA quadruplex and the PBS is ready for the next priming event.

More specifically, the present Example demonstrated separated steps of exponential DSPA. Based on the information disclosed in this Example, we have developed an isothermal, exponential and cost-effective assay for DNA signal amplification. The assay allows an unprecedented 1010-fold amplification of DNA signal in less than 40 min.

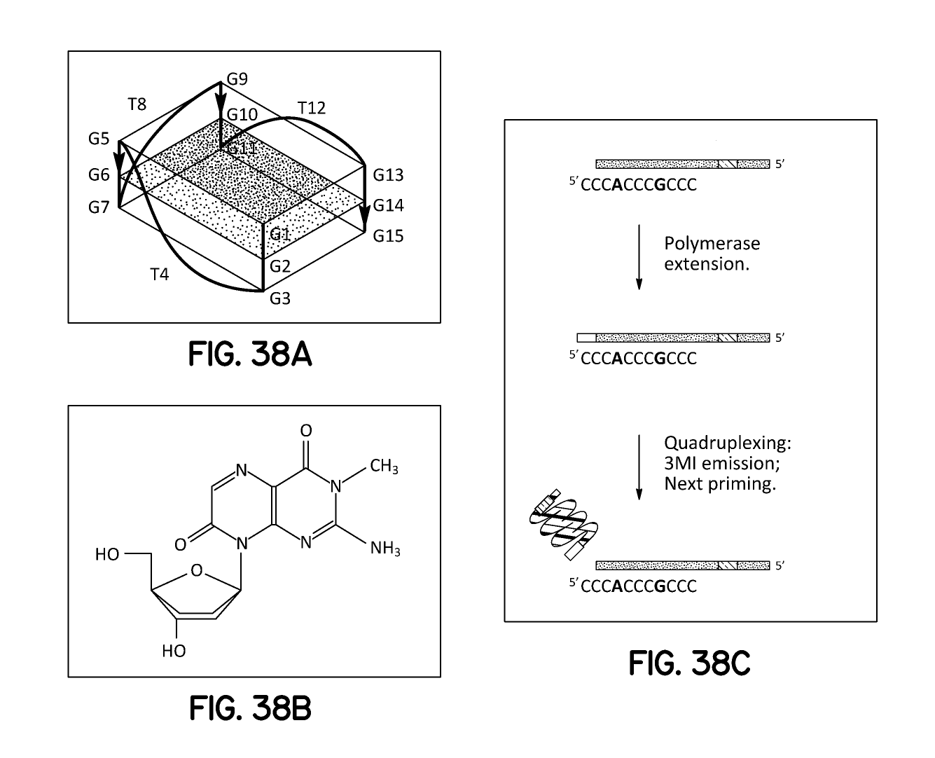

As discussed above, the free energy of DNA quadruplexes (and/or other dissociative structures) can be used to drive unfavorable (endergonic) reactions, which led to the development of dissociative structure priming amplification (DSPA) [See also Kankia, B. I. (2011) Self-dissociative primers for nucleic acid amplification and detection based on DNA quadruplexes with intrinsic fluorescence. Analytical biochemistry, 409, 59-65, incorporated by reference herein in its entirety]. The key point of quadruplex-driven reactions is that the GGGTGGGTGGGTGGG [SEQ. ID. NO. 14] (G3T) sequence is capable of forming a monomolecular quadruplex structure (see FIG. 38) with unusually high thermal stability. In contrast, the starting primer, which is a truncated version of G3T, is missing a guanine residue critical for quadruplex formation. As a result the truncated sequence anneals to the PBS without complication. When the polymerase adds the missing guanine, the extended primer spontaneously folds into a DNA quadruplex and the PBS is ready for the next primer binding/extension event (FIG. 38). In addition, primers containing fluorescent nucleotides in the loop positions demonstrated a strong increase in fluorescence upon quadruplex formation, which permits simple and effective quantification without extra probe molecules [see Kankia, B. I. (2011) Self-dissociative primers for nucleic acid amplification and detection based on DNA quadruplexes with intrinsic fluorescence. Analytical biochemistry, 409, 59-65; Gogichaishvili, S., Johnson, J., Gvarjaladze, D., Lomidze, L. and Kankia, B. (2013) Isothermal amplification of DNA using quadruplex primers with fluorescent pteridine base analogue 3-methyl isoxanthopterin. Biopolymers; Taylor, A., Joseph, A., Okyere, R., Gogichaishvili, S., Musier-Forsyth, K. and Kankia, B. (2013) Isothermal quadruplex priming amplification for DNA-based diagnostics. Biophysical chemistry, 171, 1-8, incorporated by reference herein in their entireties].

Thus, DSPA takes advantages of two unique properties of the G3T quadruplex—thermodynamic (unusually high stability) and optical (emission of fluorescence bases). While the former allows plateau-free and truly isothermal amplification [Kankia, B. I. (2011) Self-dissociative primers for nucleic acid amplification and detection based on DNA quadruplexes with intrinsic fluorescence. Analytical biochemistry, 409, 59-65; Gogichaishvili, S., Johnson, J., Gvarjaladze, D., Lomidze, L. and Kankia, B. (2013) Isothermal amplification of DNA using quadruplex primers with fluorescent pteridine base analogue 3-methyl isoxanthopterin. Biopolymers; Taylor, A., Joseph, A., Okyere, R., Gogichaishvili, S., Musier-Forsyth, K. and Kankia, B. (2013) Isothermal quadruplex priming amplification for DNA-based diagnostics. Biophysical chemistry, 171, 1-8 incorporated by reference herein in their entireties], the latter creates several additional advantages for DNA signal amplification. First, DSPA simplifies the reaction mixture by eliminating separate probe molecules (i.e., TaqMan, Molecular Beacons). Second, signal production in DSPA is a monomolecular process, which results in immediate signal from the very first quadruplex. In contrast, the use of existing bimolecular detection mechanisms makes hybridization between amplicons and probes entropically unfavorable, which restricts detection at initial cycles of amplification and slows down detection at later cycles. Third, the intrinsic detection mechanism of DSPA allows for further simplification of the assay by complete separation of the recognition step from the signal amplification. In contrast, all current detection mechanisms require the presence of probe molecules in the amplification reaction, since fluorescence signal is created upon hybridization of probes to amplicons. The latter not only complicates the reaction, it also requires costly double-attachment for each target, which significantly increases expenses. The modularity in DSPA allows for the designing of a universal signal amplification system, which can amplify signal from any biomarker. This allows for low-cost multiple diagnostics of DNA samples attached to magnetic beads using as single fluorescence signal and can be easily adapted to nucleic acid extraction cassettes [Bordelon, H., Adams, N. M., Klemm, A. S., Russ, P. K., Williams, J. V., Talbot, H. K., Wright, D. W. and Haselton, F. R. (2011) Development of a low-resource RNA extraction cassette based on surface tension valves. ACS Appl Mater Interfaces, 3, 2161-2168, incorporated by reference herein in its entirety] (FIG. 23).

In this Example, we demonstrate two-primer DSPA for DNA signal amplification (FIG. 24). In this design, and as described in detail above, the DSPA primer binds to the probe (i) and initiates its replication (ii). After spontaneous quadruplex formation, the next DSPA priming occurs (iii), which is accompanied by displacement of the first amplicon (iv). The displaced strand contains a freely available primer-binding site for the second primer. The primer binds (v) and replicates the amplicon, including the quadruplex at the 5′-end (vi). After another step of amplification, a short duplex containing primer-binding sites for both primers is created and the reaction becomes exponential (right, FIG. 24). Based on this scheme, the present inventors have developed an isothermal and cost-effective assay for DNA signal amplification. Unlike previous assays, the current assay uses the free energy of a DNA tertiary structure as a driving force for the amplification and possesses an intrinsic quantification mechanism. The assay allows an unprecedented 1010-fold amplification in less than 40 min and is compatible with the requirements of point-of-care molecular diagnostics.

Materials and Methods

Enzymes and DNA Substrates

All DNA polymerases and dNTPs were purchased from New England BioLabs. All unmodified and 2AP-containing oligonucleoitdes were obtained from Integrated DNA Technologies. 3MI-containing primers were purchased from Fidelity Systems. The concentration of DNA oligonucleotides was determined by measuring UV absorption at 260 nm as described earlier [Kankia, B. I. and Marky, L. A. (1999) DNA, RNA, and DNA/RNA oligomer duplexes: a comparative study of their stability, heat, hydration and Mg(2+) binding properties. J Phys Chem. B, 103, 8759-8767, incorporated by reference herein in its entirety]. All experiments were performed in the buffer conditions suitable for DNA polymerases: 50 mM monovalent cations (K+ and Cs+), 2 mM MgCl2, 10 mM Tris-HCl, pH 8.7.

Fluorescence Measurements

Fluorescence measurements of 2AP (Ex 310 nm, Em 370 nm) and 6MI (Ex 340 nm, Em 430 nm) were performed using a Varian spectrophotometer (Cary Eclipse), a microplate reader FluoDia T70 (Photon Technology International) and ESEQuant Tube Scanner (Qiagen). Linear DSPA, quadruplex thermal unfolding and quadruplex unfolding by polymerases were carried out directly in the quartz cuvettes using the spectrophotometer. Exponential DSPA were performed in microplates and 0.2 mL PCR tubes using a plate reader and tube scanner, respectively.

Linear DSPA Protocol

Linear DSPA reactions were carried out in a reaction mixture containing 1 μM primer, 1 nM target, 800 μM dNTP, buffer (2 mM MgCl₂, 25 mM CsCl, 25 mM KCl, 10 mM Tris-HCl, pH 8.7) and 0.05 U/μl Taq. The reactions were carried out directly in the quartz cuvettes. The solution was vortexed for 2-3 seconds and immediately inserted into a cell holder of the fluorometer equilibrated at reaction temperature followed by real-time monitoring of 2AP fluorescence. DSPA rates were determined from the initial slopes of the kinetic curves conducted at different temperatures. After each experiment, the cuvettes were washed with DNAZap solutions (Invitrogen) for complete elimination of DNA products.

Unfolding of G3T Quadruplex by DNA Polymerases

For the polymerase unfolding experiments, 99 μl reaction mixtures containing 1 μM template with quadruplex, 1.1 μM primer, and 800 μM dNTP were incubated at desired temperatures for 2-5 min directly in the cuvettes inserted in the cell holders of the fluorometer. After the preincubation time, cuvettes were removed from the cell holders, the 1 μl DNA polymerase solution of 5 U/μl concentration was added, mixed by pipetman, inserted back into the fluorometer and real-time monitoring of 2AP fluorescence was initiated immediately. In the control experiments performed in the absence of the polymerases, 1 μl buffer was added to the reaction mixture.

Exponential DSPA Protocol

Exponential DSPA amplifications were carried out in a 100 μl reaction mixture containing buffer, template, left primer, DSPA primer and dNTP. The reaction mixtures were incubated at reaction temperatures for 1 min. After preincubation time, DNA polymerase was added and real-time monitoring of 3MI fluorescence was initiated immediately. In the case of the plate reader, mixing was performed by pipetman directly in the microplates, while in the case of the tube scanner, mixing was performed by vortexing the solutions for 2-3 seconds in 0.2 mL PCR tubes. The concentrations of the reaction components are given in Figure captions. The desired amount of template was prepared by 10-fold serial dilutions with the final concentrations ranging between 100 μM and 100 aM in eppendorf tubes. The solutions were used immediately to avoid sticking of the templates to the tubes.

Results and Discussion

Design of the Probe and Primers for Exponential DSPA

In order to design an efficient DSPA diagnostic tool, each step of the reaction (shown in FIG. 24) should be optimized. While most of the steps require suitable experimental conditions (i.e. temperature or buffer for a given polymerase), two steps (iii and vi) need optimization. These steps are responsible for the isothermal and plateau-free nature of DSPA and are discussed below (see sections “DSPA priming” and “Quadruplex invading replication”). Since fluorescent nucleotides are part of the primers, they must be replicated to create a full-length PBS in newly generated amplicons. However, DNA polymerases demonstrate different levels of tolerance to the fluorescent nucleotides [Gogichaishvili, S., Johnson, J., Gvarjaladze, D., Lomidze, L. and Kankia, B. (2013) Isothermal amplification of DNA using quadruplex primers with fluorescent pteridine base analogue 3-methyl isoxanthopterin. Biopolymers; Taylor, A., Joseph, A., Okyere, R., Gogichaishvili, S., Musier-Forsyth, K. and Kankia, B. (2013) Isothermal quadruplex priming amplification for DNA-based diagnostics. Biophysical chemistry, 171, 1-8, incorporated by reference herein in their entireties] which requires further consideration in probe design and is discussed in the following section.

Selection of the Fluorescent Nucleotide

Fluorescent nucleotide analogs for amplicon quantification are highly desired since they can be readily incorporated into oligonucleotides during solid-phase synthesis. There are several suitable base analogs (i.e., 2-aminopurine (2AP) Ex310, Em370; 6-methylisoxanthopterin (6MI) Ex340, Em430; and 3-methylisoxanthopterin (3MI) Ex348, Em431) [Law, S. M., Eritja, R., Goodman, M. F. and Breslauer, K. J. (1996) Spectroscopic and calorimetric characterizations of DNA duplexes containing 2-aminopurine. Biochemistry, 35, 12329-12337; McLaughlin, L. W., Leong, T., Benseler, F. and Piel, N. (1988) A new approach to the synthesis of a protected 2-aminopurine derivative and its incorporation into oligodeoxynucleotides containing the Eco RI and Bam HI recognition sites. Nucleic acids research, 16, 5631-5644; Hawkins, M. E. (2008) Fluorescent pteridine probes for nucleic acid analysis. Methods in enzymology, 450, 201-231; Hawkins, M. E. (2007) Synthesis, purification and sample experiment for fluorescent pteridine-containing DNA: tools for studying DNA interactive systems. Nat Protoc, 2, 1013-1021; Hawkins, M. E., Pfleiderer, W., Balis, F. M., Porter, D. and Knutson, J. R. (1997) Fluorescence properties of pteridine nucleoside analogs as monomers and incorporated into oligonucleotides. Analytical biochemistry, 244, 86-95, incorporated by reference herein in their entireties]. Although these fluorescent bases have the potential to be sensitive probes, their fluorescence is significantly quenched upon incorporation into oligonucleotides, which makes them impractical in RT-PCR [Kourentzi, K. D., Fox, G. E. and Willson, R. C. (2003) Hybridization-responsive fluorescent DNA probes containing the adenine analog 2-aminopurine. Analytical biochemistry, 322, 124-126; Marti, A. A., Jockusch, S., Li, Z., Ju, J. and Turro, N. J. (2006) Molecular beacons with intrinsically fluorescent nucleotides. Nucleic acids research, 34, e50, incorporated by reference herein in their entireties]. In contrast, we have shown that fluorescent base analogs incorporated at loop positions of G3T demonstrate a remarkable increase in fluorescence due to becoming fully accessible to solvent upon quadruplex formation (lit). 2AP is a fluorescent analog of adenine that forms Watson-Crick base-pairs with thymidine [Law, S. M., Eritja, R., Goodman, M. F. and Breslauer, K. J. (1996) Spectroscopic and calorimetric characterizations of DNA duplexes containing 2-aminopurine. Biochemistry, 35, 12329-12337; McLaughlin, L. W., Leong, T., Benseler, F. and Piel, N. (1988) A new approach to the synthesis of a protected 2-aminopurine derivative and its incorporation into oligodeoxynucleotides containing the Eco RI and Bam HI recognition sites. Nucleic acids research, 16, 5631-5644, incorporated by reference herein in their entireties] and is well tolerated by DNA polymerases [Fidalgo da Silva, E., Mandal, S. S. and Reha-Krantz, L. J. (2002) Using 2-aminopurine fluorescence to measure incorporation of incorrect nucleotides by wild type and mutant bacteriophage T4 DNA polymerases. The Journal of biological chemistry, 277, 40640-40649, incorporated by reference herein in its entirety]. 6MI and 3MI are structurally similar to guanine. While 6MI almost perfectly base pairs with cytosine, 3MI does not pair with natural nucleic-acid bases [Hawkins, M. E. (2008) Fluorescent pteridine probes for nucleic acid analysis. Methods in enzymology, 450, 201-231; Hawkins, M. E., Pfleiderer, W., Mazumder, A., Pommier, Y. G. and Balis, F. M. (1995) Incorporation of a fluorescent guanosine analog into oligonucleotides and its application to a real time assay for the HIV-1 integrase 3′-processing reaction. Nucleic acids research, 23, 2872-2880 incorporated by reference herein in their entireties]. Neither nucleotide is tolerated by DNA polymerases [Taylor, A., Joseph, A., Okyere, R., Gogichaishvili, S., Musier-Forsyth, K. and Kankia, B. (2013) Isothermal quadruplex priming amplification for DNA-based diagnostics. Biophysical chemistry, 171, 1-8; Datta, K., Johnson, N. P., Villani, G., Marcus, A. H. and von Hippel, P. H. (2012) Characterization of the 6-methyl isoxanthopterin (6-MI) base analog dimer, a spectroscopic probe for monitoring guanine base conformations at specific sites in nucleic acids. Nucleic acids research, 40, 1191-1202 incorporated by reference herein in their entireties]. 3MI (as shown in FIG. 38B) was used in the present Example due to its (i) longer excitation wavelength (348 nm); (ii) high quantum yield (0.88); and (iii) ability to serve as efficient terminator for polymerase activity, which allows the use of an 11-nt DSPA PBS (see FIG. 38) and increased efficiency of DSPA [Gogichaishvili, S., Johnson, J., Gvarjaladze, D., Lomidze, L. and Kankia, B. (2013) Isothermal amplification of DNA using quadruplex primers with fluorescent pteridine base analogue 3-methyl isoxanthopterin. Biopolymers, incorporated by reference herein in its entirety].

DSPA Priming

The temperature dependence of linear DSPA (FIG. 38C), which requires DNA polymerase to add only one guanine to the primer, was carefully studied earlier [Gogichaishvili, S., Johnson, J., Gvarjaladze, D., Lomidze, L. and Kankia, B. (2013) Isothermal amplification of DNA using quadruplex primers with fluorescent pteridine base analogue 3-methyl isoxanthopterin. Biopolymers, incorporated by reference herein in its entirety]. The results indicate that the rate of linear DSPA closely correlates with the melting behavior of the primer/PBS complexes and reveals the optimal rate at temperatures 10-15° C. higher than the Tm of primer/template complex [Gogichaishvili, S., Johnson, J., Gvarjaladze, D., Lomidze, L. and Kankia, B. (2013) Isothermal amplification of DNA using quadruplex primers with fluorescent pteridine base analogue 3-methyl isoxanthopterin. Biopolymers, incorporated by reference herein in its entirety]. In linear DSPA (FIG. 38C), the quadruplex does not have any attachments, which facilitates its dissociation from the target. However, in exponential DSPA, the incoming primer should be able to displace the newly formed quadruplex, which is part of a longer amplicon (step (iii), FIG. 24).

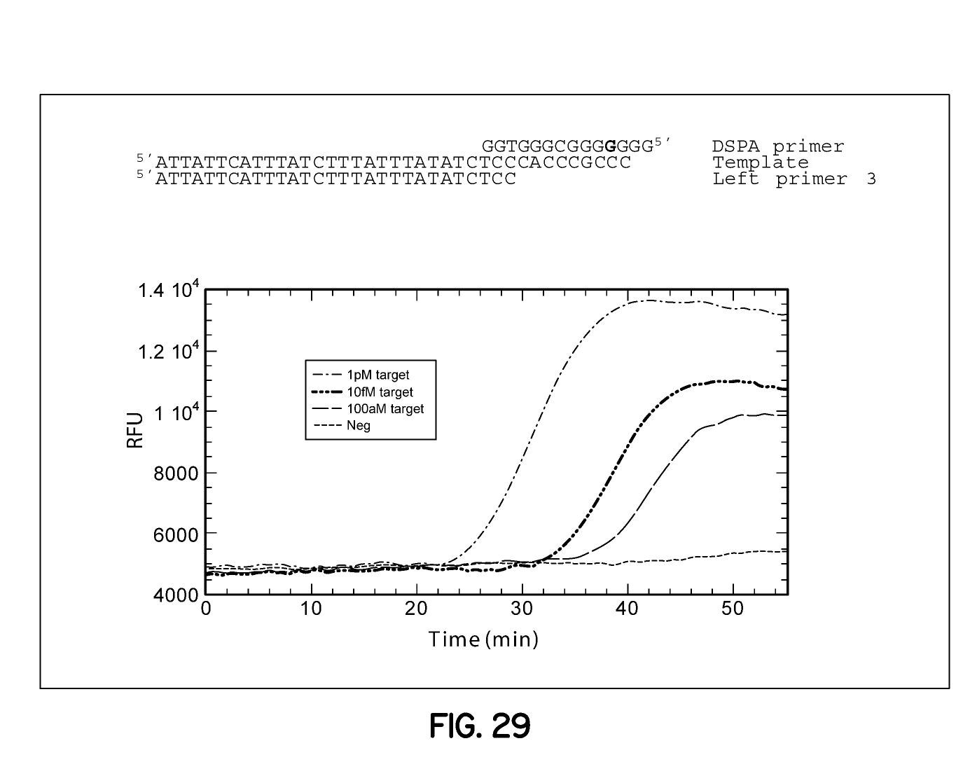

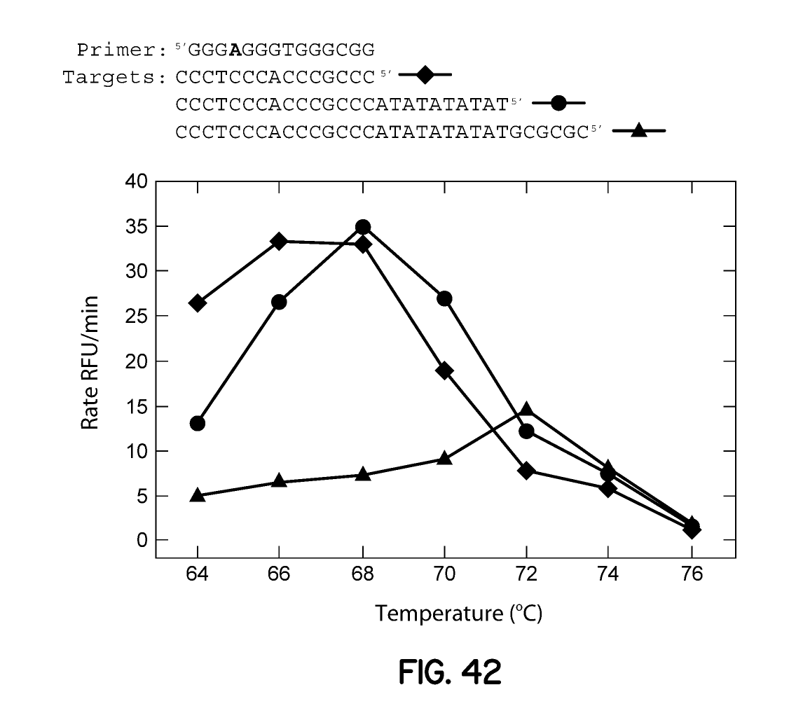

To test whether primer displacement takes place in longer amplicons, we studied the DSPA rate-dependence for a series of DNA sequences (FIG. 42). The shortest target revealed a typical DSPA behavior with the maximum rate observed at ˜67° C. [Taylor, A., Joseph, A., Okyere, R., Gogichaishvili, S., Musier-Forsyth, K. and Kankia, B. (2013) Isothermal quadruplex priming amplification for DNA-based diagnostics. Biophysical chemistry, 171, 1-8, incorporated by reference herein in its entirety]. Attachment of a 10-nt AT-containing tail to the target sequence did not affect DSPA significantly. The entire curve shifted slightly to higher temperatures showing a maximum rate at 68° C. (FIG. 42). This low stability, despite the presence of a 10-nt AT tail, is a very useful feature, which we used in designing two-primer exponential DSPA shown in FIG. 28. In contrast, the addition of 6 extra GC base pairs significantly decreased the overall DSPA rate and a peak is observed at 72° C. Thus, primer-displacement is significantly inhibited by the increased thermal stability of a 31-bp duplex with GC-rich ends (FIG. 42). Thus, AT-rich sequences attached to targets do not affect the DSPA rate significantly. In contrast, when longer and more stable amplicons are used, DSPA should be conducted at 4-5° C. higher than the most favorable temperature observed for DSPA with the short PBS.

Quadruplex Invasion Replication

Another challenge of isothermal and exponential DSPA is that two opposite processes, quadruplex folding upon DSPA primer elongation and quadruplex unfolding upon left primer elongation, should take place under the same experimental conditions. By increasing concentration of K+ ions, the present inventors determined that they can facilitate the former and impede the latter, or vice versa.

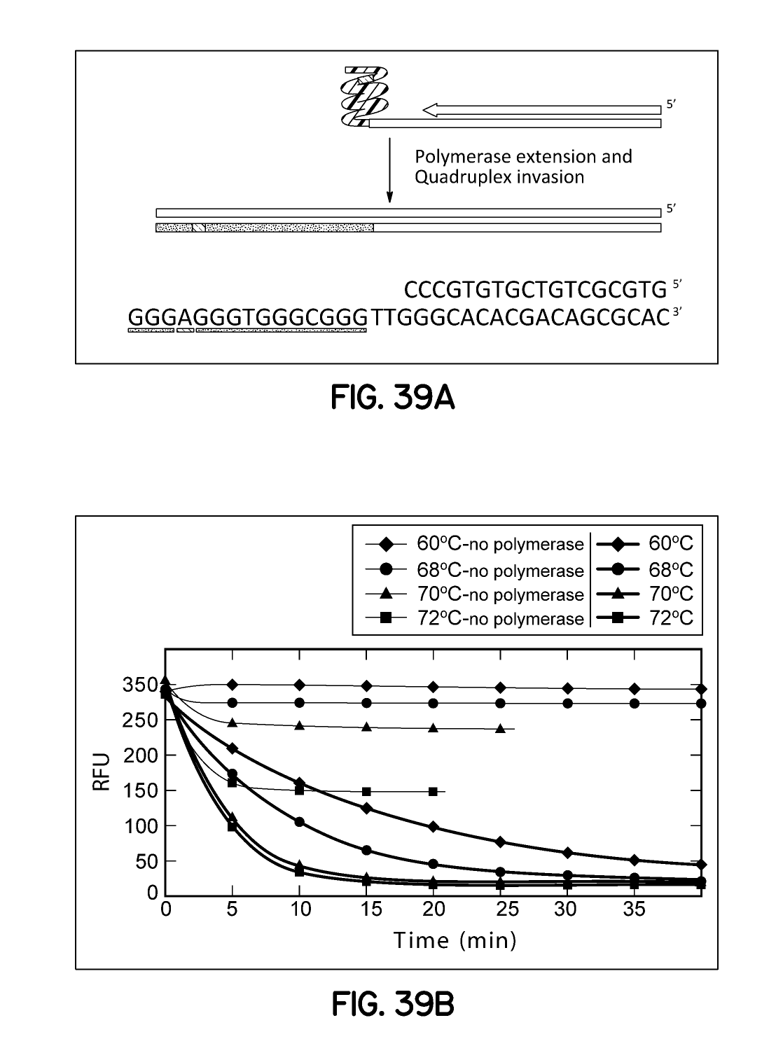

The role of K+ ions in linear DSPA was studied earlier, which revealed optimal activity at 25 mM concentration [Taylor, A., Joseph, A., Okyere, R., Gogichaishvili, S., Musier-Forsyth, K. and Kankia, B. (2013) Isothermal quadruplex priming amplification for DNA-based diagnostics. Biophysical chemistry, 171, 1-8, incorporated by reference herein in its entirety]. However, measurable activity was observed between 5 and 50 mM K+ concentration. The ability of DNA polymerases to unfold the quadruplex has been studied by quadruplex-based assay (FIG. 39).

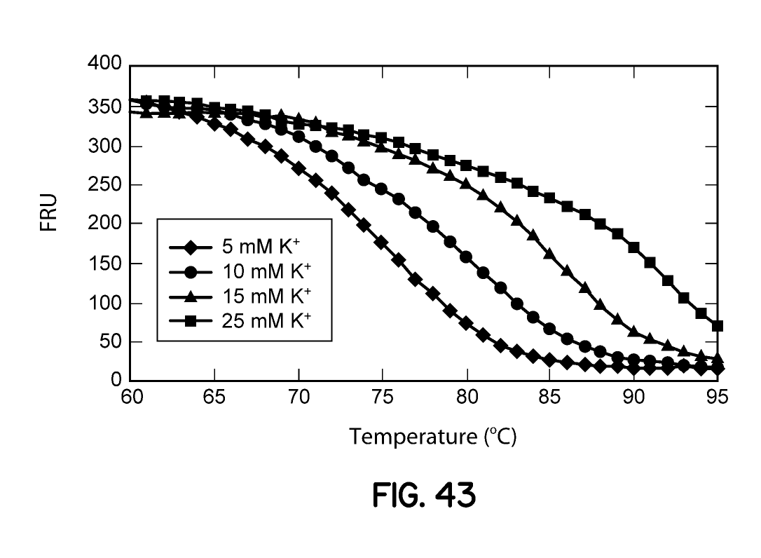

In this assay, a DNA quadruplex containing 2AP is attached to a 20-nt long sequence, which contains the PBS for an 18-nt primer (FIG. 39A). The template replication will only occur upon quadruplex unfolding, which should be accompanied by decrease in fluorescence due to the quenching of 2AP by neighboring guanines. To be sure that the quadruplexes are intact in these experiments, fluorescence thermal unfolding experiments were performed in the presence of varying amount of K+ ions (FIG. 43). As expected, quadruplex stability strongly depends on the concentration of K+ ions. However, they are securely folded below 65° C. even in the presence of 5 mM K+ ions (FIG. 43). Initially, quadruplex unfolding by Taq polymerase was studied in the presence of 5 mM K+ ions at 60, 68, 70 and 74° C. (FIG. 39B). The control experiments (thin lines), in which plain buffer was added into reaction mixtures instead of polymerase, estimate possible destabilization effects due to the temperature. At 60 and 68° C. we did not observe any significant destabilization (black and red thin lines). However, at 70 and 74° C., around 20% and 50% of the quadruplexes, respectively, are unfolded in the absence of the polymerases (blue and green thin lines, FIG. 39B). These data are in good agreement with thermal unfolding experiments of the quadruplex (FIG. 43).

Thus, at 60° C. and 68° C., fluorescence quenching (black and red thick lines) is induced solely by polymerase activity. The unfolding rates estimated from the initial slopes are ˜50 nM/min and ˜80 nM/min, respectively. At higher temperatures, 70° C. and 74° C., 80% and 50% of the total quenching effects are due to the polymerase activity (blue and green thick lines, FIG. 39B). Interestingly, the unfolding rates are the same at both temperatures, ˜200 nM/min, which indicates that above 70° C., quadruplex unfolding is not the rate-limiting step anymore.

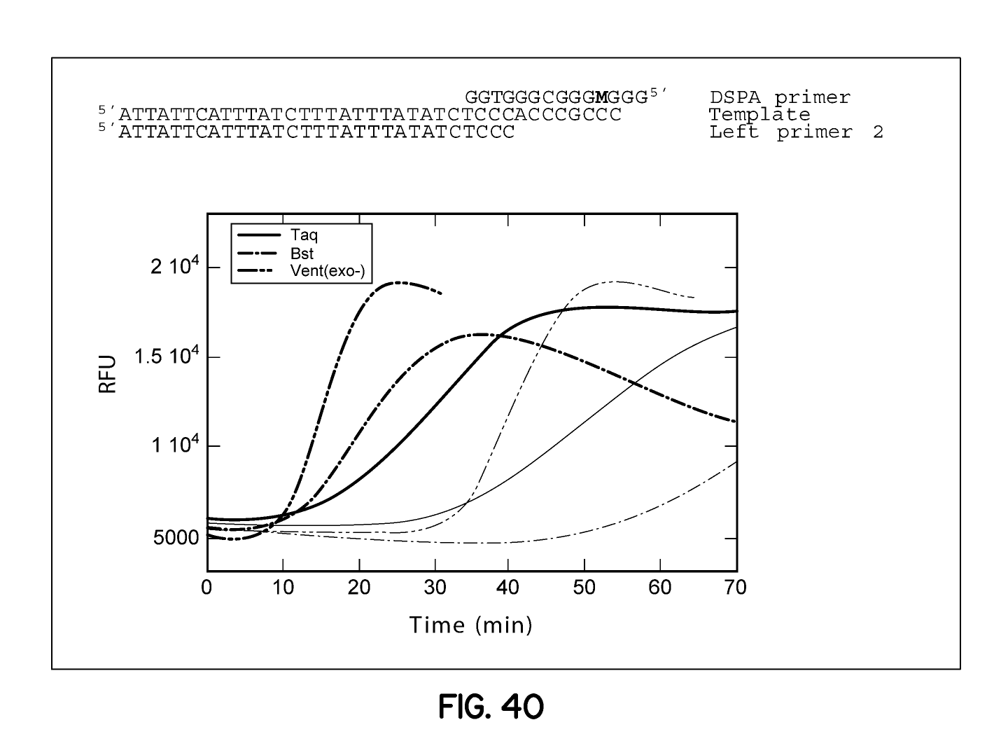

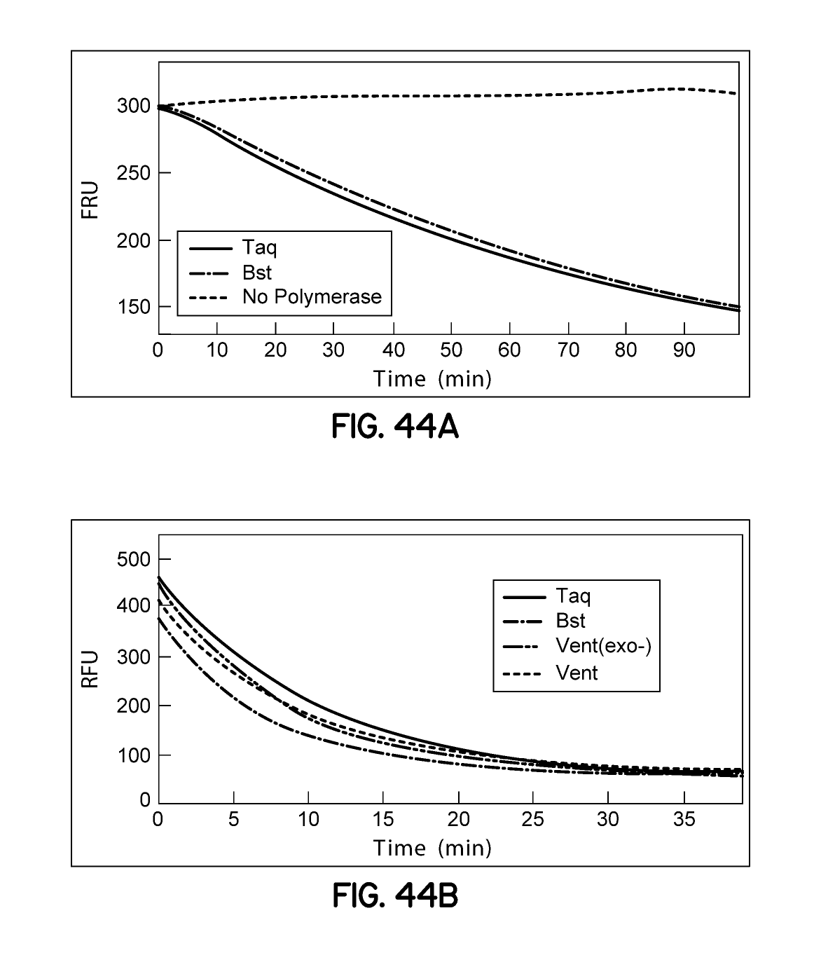

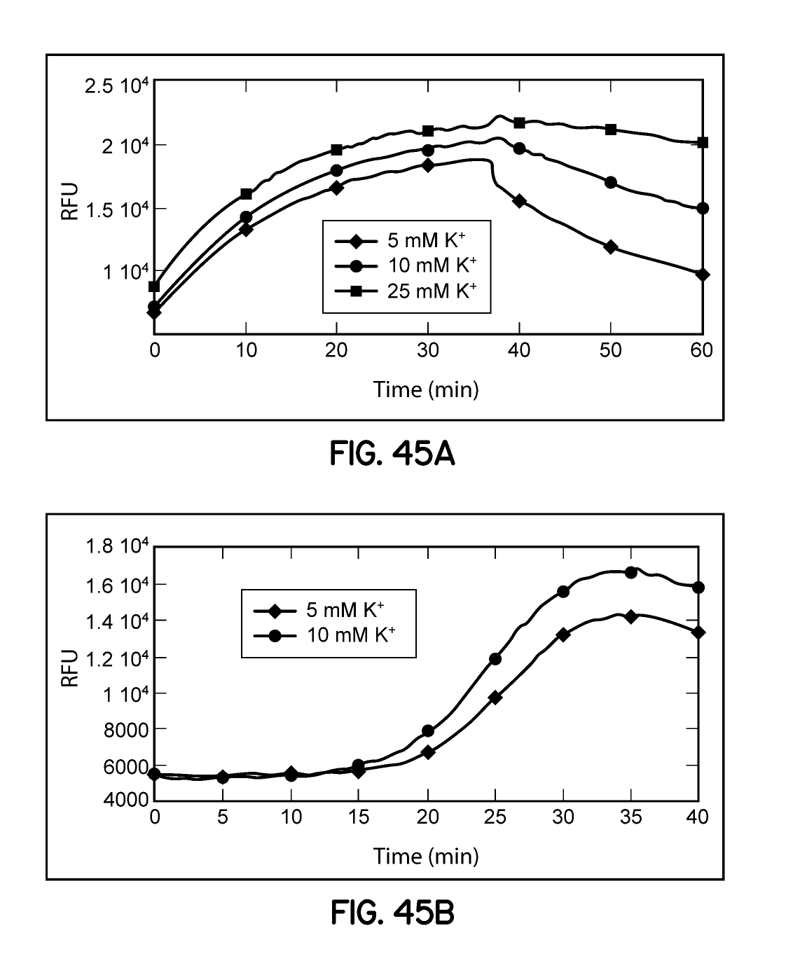

Additional data, collected for different DNA polymerases under various experimental conditions, show that all polymerases studied here reveal similar quadruplex unfolding activity including (FIG. 44). The polymerases are able to unfold the quadruplexes quite efficiently even at 25 mM K+ ions (FIG. 44A). However, the rate of the unfolding process depends on the temperature and concentration of K+ ions. For the final tuning of the experimental conditions, we studied amplification of the actual template (FIG. 40) as a function of K+ concentration, which revealed that the most efficient exponential DSPA in the presence of 10 mM K+(FIG. 45).

Exponential DSPA

The template (FIG. 40), which is constructed according to the studies above, represents a conjugate of AT-rich left primer and 11-nt DSPA PBS [Gogichaishvili, S., Johnson, J., Gvarjaladze, D., Lomidze, L. and Kankia, B. (2013) Isothermal amplification of DNA using quadruplex primers with fluorescent pteridine base analogue 3-methyl isoxanthopterin. Biopolymers, incorporated by reference herein in its entirety]. Several important features of the template should be noted. First, the left primer is AT-rich to keep thermal stability of the product DNA as low as possible (see Table 1, below). Second, for the same reason, DSPA is based on 11-nt PBS [Gogichaishvili, S., Johnson, J., Gvarjaladze, D., Lomidze, L. and Kankia, B. (2013) Isothermal amplification of DNA using quadruplex primers with fluorescent pteridine base analogue 3-methyl isoxanthopterin. Biopolymers, incorporated by reference herein in its entirety]. Third, the primers can overlap to (i) facilitate quadruplex invasion by the left primer, (ii) stabilize the left primer/template complex without undesired stabilization of the product DNA; and (iii) create a simple internal positive control of DSPA. The experiments were set at 66° C. where primers can be efficiently elongated [Gogichaishvili, S., Johnson, J., Gvarjaladze, D., Lomidze, L. and Kankia, B. (2013) Isothermal amplification of DNA using quadruplex primers with fluorescent pteridine base analogue 3-methyl isoxanthopterin. Biopolymers, incorporated by reference herein in its entirety], while the product DNA is significantly destabilized to allow spontaneous quadruplex formation (see Table 1, below).

TABLE 1
Melting temperatures (Tm) of DNA duplexes involved in
the exponential DSPA.

| Complex | Cs+ | K+ |
|---|---|---|
| Template/DSPA primer | 60.4 (53.3) | 60.5 (53.3) |
| Template/Left primer | 62.0 (59.0) | 58.5 (55.5) |
| Product | 71.0 (68.8) | 67.0 (64.8) |

DNA sequences are shown in FIG. 40. The Tm values are obtained from UV melting performed at 6 μM strand concentration in Cs+(50 mM CsCl, 2 mM MgCl₂) and K+(10 mM KCl, 40 mM CsCl, 2 mM MgCl₂) buffers. Values in parentheses correspond to estimations at 500 nM strand concentration.

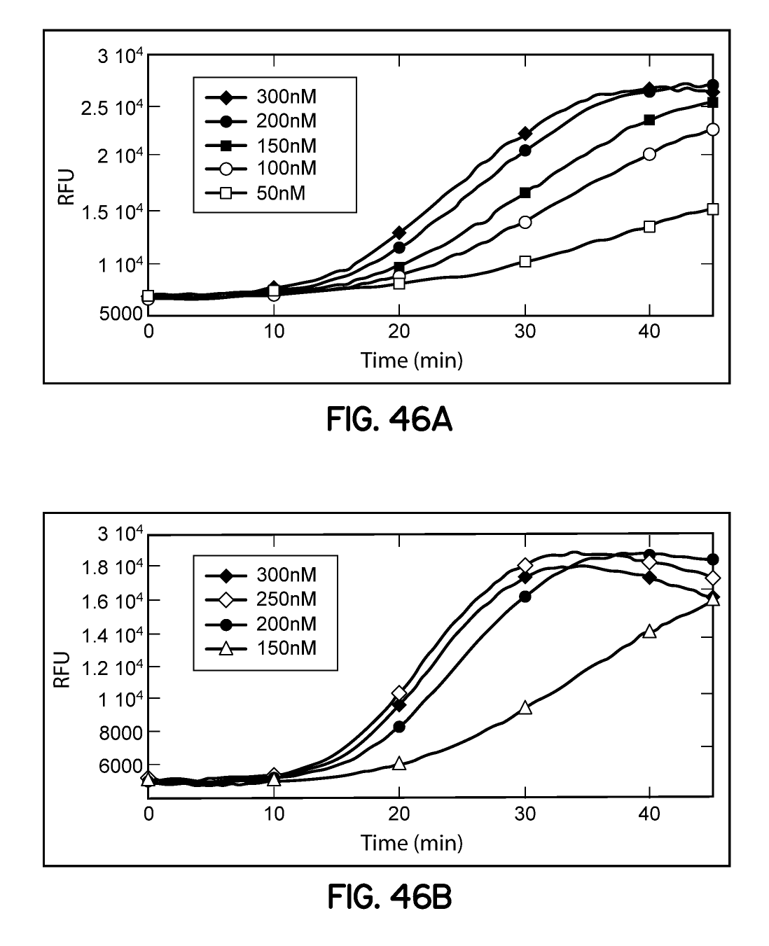

In the exponential DSPA, described in FIG. 24, the quantification is performed through the fluorescence of the quadruplex, which constantly folds (emits light) and unfolds (quenched) upon amplification. As discussed above, quadruplex unfolding, which is accompanied by fluorescence quenching, is required to have an exponential amplification. Thus, it is a consideration to select the proper ratio of the primers, to allow robust but measurable fluorescence signal. Therefore, we performed series of DSPA reactions at different primer concentrations (FIG. 46). In these experiments we kept a constant concentration of DSPA primer and varied the amount of the left primer. The study revealed that the best activity, under the given experimental conditions, was observed when the amount of the left primer was 15-20% lower than DSPA primer (see FIG. 46).

To find an appropriate polymerase for the exponential DSPA, we performed another series of experiments. In these experiments, two kinetic profiles were recorded for each polymerase: (i) amplification in the presence of 100 μM template and (ii) negative control, which contains all reaction components except for the template (FIG. 40). The fastest and most efficient amplification was observed for Vent (exo-). The fluorescence arises at ˜8 min and reaches the maximum level in less than 15 min. The negative control demonstrates strong background activity, which repeats the profile of the amplification curve but with a ˜25 min delay. In the case of Bst 2.0, positive amplification displays a fluorescence increase after 10 min (FIG. 40), however, (i) the slope is significantly smaller; (ii) the signal never reaches the maximum level; and (iii) quenching is revealed after 35 min. The negative control of Bst 2.0 reveals weak background activity only after 50 min. Taq polymerase demonstrates the slowest increase after 10 min, leveling off at ˜45 min, and its negative control reveals similar amplification pattern with ˜20 min delay. The experiments, presented in FIG. 40, demonstrate that all three polymerases can be used for DSPA. However, for the next step of the experiments we selected Vent (exo-) since it reveals the most rapid amplification.

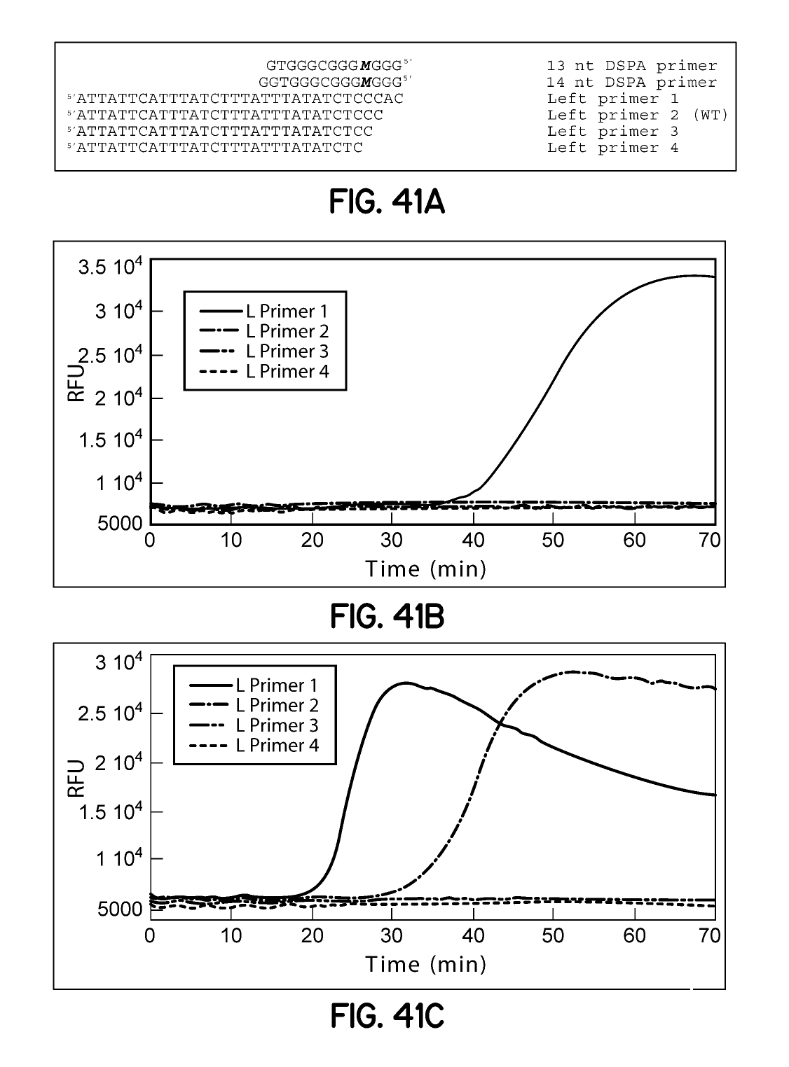

Before recording DSPA amplification as a function of the template concentration, we performed the last series of experiments to reveal the reason for the background amplification. In these experiments we used a set of primers based on the sequences shown in FIG. 40. Specifically, we studied four left primers and two DSPA primers with different length of overlaps between them (FIG. 41A). For instance, the 13-nt DSPA primer is able to overlap with the left primers by 0-, 1- and 3-nt. The corresponding experiments show that the primer pair with 3-nt overlap (left primer 1 and 13-nt DSPA primer) has background activity (FIG. 41B). All other primer pairs, including 1-nt overlap, display horizontal lines (FIG. 41B). In the case of the 14-nt DSPA primer, which forms 0-, 1-, 2- and 4-nt overlaps, we observe background amplifications for 2- and 4-nt overlaps (FIG. 41C). The primer pair with the 4-nt overlap starts rapid increasing at 20 min and reaches the maximum fluorescence level in 10 min. As expected, the primer pair with shorter overall, 2-nt, demonstrates delayed background activity at 30 min (FIG. 41C). This study clearly indicates that the background amplification in the exponential DSPA is due to overlapping between the primers. Thus, by employing primers with 2-nt overlap we can introduce a very simple positive control for DSPA without any extra components. Note that to exaggerate the possible background activity, primer concentrations were increased as indicated in the caption of FIG. 41.

In the final test, DSPA amplification was recorded as a function of probe concentration. We tested three different systems:

First System Tested

First, the constructs described in FIG. 40 with 2-nt overlap. FIG. 28A shows representative curves of the system conducted at different concentrations of the probe molecule, and FIG. 28B demonstrates a correlation between time and the logarithm of the probe concentration. The template (shown at the top of FIG. 28) represents a conjugate of AT-rich left primer and 11-nt DSPA PBS. Several features of the template may be noted. First, the left primer is AT-rich to keep thermal stability of the product DNA as low as possible. Second, for the same reason, DSPA is based on 11-nt PBS. Third, the primers can overlap to (i) facilitate quadruplex invasion by the left primer, (ii) stabilize the left primer/template complex without undesired stabilization of the product DNA; and (iii) create a simple internal positive control of DSPA. The experiments were set at 66° C. where primers can be efficiently elongated, while the product DNA is significantly destabilized to allow spontaneous quadruplex formation. FIG. 28, panel A, shows representative curves of the system conducted at different concentrations of the probe molecule, and FIG. 28, panel B, demonstrates a correlation between time and the logarithm of the probe concentration. The dependence is linear from 100 μM to 10 fM. However, at lower concentrations (1 fM and 100 aM) points deviate from the linear dependence. This system demonstrates background activity due to 2-nt overlap.

Second System Tested

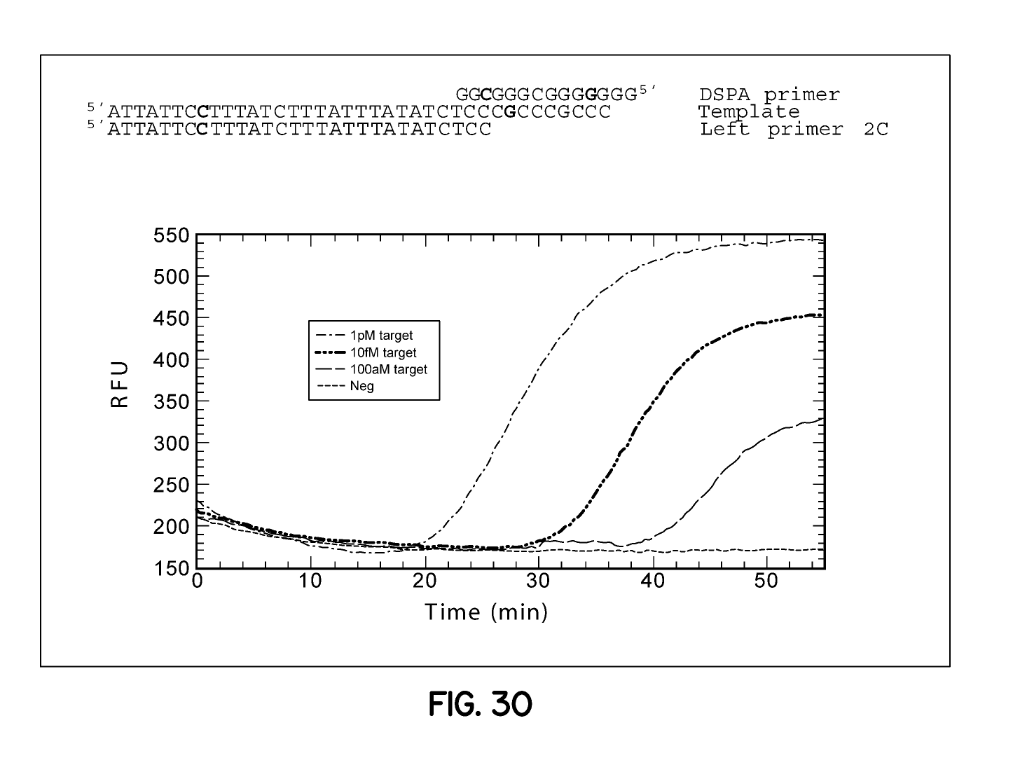

The second system is similar to the previous DSPA. Only difference is that the left primer is truncated resulting to 1-nt overlap between the primers (FIG. 29). The system does not display any background activity, while the 100 aM template is easily detectable.

Third System Tested

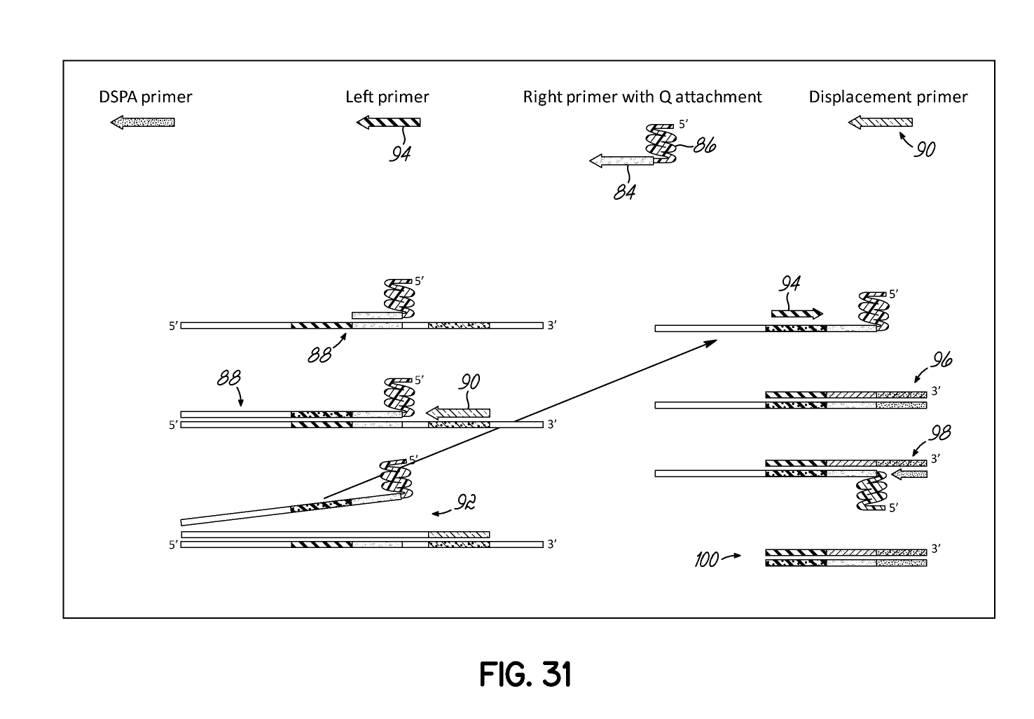

In the last system (FIG. 30) the primers overlap with 1-nt, but the stability of the primer/PBS complexes are increased by the T→C substitution in the DSPA primer and the A→C substitution in the left primer (shown in red). As a result, the optimal temperature for the amplification shifts to 69° C. Similarly to the previous system, this DSPA also is free from the background activity.

The embodiments of the present invention recited herein are intended to be merely exemplary and those skilled in the art will be able to make numerous variations and modifications to it without departing from the spirit of the present invention. Notwithstanding the above, certain variations and modifications, while producing less than optimal results, may still produce satisfactory results. All such variations and modifications are intended to be within the scope of the present invention as defined by the claims appended hereto.

What is claimed is:

1. A process for detecting the presence of a target nucleic acid by amplifying at least one target nucleic acid sequence contained in a nucleic acid or a mixture of nucleic acids, the process comprising:
bringing a nucleic acid or a mixture of nucleic acids into contact with a probe, said probe including:
a first segment having a sequence that is complementary to a target nucleic acid, and
a second segment having a sequence complementary to the sequence of a primer wherein the sequence of the primer is adapted to conform into a conformation having a free energy with more favorable thermodynamics than a corresponding B-DNA duplex; and
bringing said nucleic acid or mixture of nucleic acids into contact with said primer, wherein said primer will bind said second segment of said probe and will subsequently conform into said conformation having a free energy with more favorable thermodynamics than a corresponding B-DNA duplex during an extension step of a polymerase chain reaction under isothermal conditions.

2. The process of claim 1, wherein the conformation having a free energy with more favorable thermodynamics than a corresponding B-DNA duplex is a quadruplex.

3. The process of claim 1, the primer having a G content of an amount to conforms into a quadruplex structure during an extension step of a polymerase chain reaction.

4. The process of claim 3, wherein the G content is equal to or greater than 70%.

5. The process of claim 1, wherein at least a portion of the primer has a sequence based on (G₃₊N₁₋₇G₃₊N₁₋₇G₃₊N₁₋₇G₃₊).

6. The process of claim 1, wherein at least a portion of the primer has a sequence based on (GGGT)₄ [SEQ. ID. NO. 5].

7. The process of claim 1, the primer further including in its sequence at least one label chosen from 2Ap, 3MI, 6MI, and 6AMP.

8. The process of claim 1, wherein the probe further includes a third segment having a sequence adapted to conform into a conformation having a free energy with more favorable thermodynamics than a corresponding B-DNA duplex, wherein said primer binds said second segment of said probe and, during an extension step of a polymerase chain reaction, will unfold said third segment of said probe.

9. The process of claim 8, wherein said third segment of said probe and said extended primer will both subsequently conform into said conformation having a free energy with more favorable thermodynamics than a corresponding B-DNA duplex during an extension step of a polymerase chain reaction under isothermal conditions.

10. (canceled)

11. (canceled)

12. (canceled)

13. The process of claim 9, wherein at least a portion of the third segment of the probe and the primer each has a sequence based on (G₃₊N₁₋₇G₃₊N₁₋₇G₃₊N₁₋₇G₃₊).

14. The process of claim 9, wherein at least a portion of the third segment of the probe and the primer each has a sequence based on (GGGT)₄ [SEQ. ID. NO. 5].

15. The process of claim 9, the third segment of the probe and the primer each further include in its sequence at least one label chosen from 2Ap, 3MI, 6MI, and 6AMP.

16. A The process of claim 1, further comprising:
bringing the nucleic acid or a mixture of nucleic acids into contact with a second probe, said second probe including a first segment having a sequence that is complementary to a second target nucleic acid, and a second segment having a sequence complementary to the sequence of a second primer, wherein the sequence of the second primer is adapted to conform into a conformation having a free energy with more favorable thermodynamics than a corresponding B-DNA duplex; and
bringing said nucleic acid or mixture of nucleic acids into contact with said second primer, wherein said second primer will bind said second segment of said second probe and will subsequently conform into said conformation having a free energy with more favorable thermodynamics than a corresponding B-DNA duplex during an extension step of a polymerase chain reaction under isothermal conditions.

17. (canceled)

18. The process of claim 16, the second segments of the first and second probes having a G content of an amount to conforms into a quadruplex structure during an extension step of a polymerase chain reaction.

19. The process of claim 18, wherein the G content is equal to or greater than 70%.

20. The process of claim 16, wherein at least a portion of the second segments of the first and second probes has a sequence based on (G₃₊N₁₋₇G₃₊N₁₋₇G₃₊N₁₋₇G₃₊).

21. The process of claim 16, wherein at least a portion of the second segments of the first and second probes has a sequence based on (GGGT)₄ [SEQ. ID. NO. 5].

22. The process of claim 16, the second segments of the first and second probes further including in its sequence at least one label chosen from 2Ap, 3MI, 6MI, and 6AMP.

23. A process for preparing a library of a target nucleic acid for sequencing by amplifying the target nucleic acid sequence, the process comprising:
bringing a nucleic acid into contact with a first primer, said first primer immobilized to a surface, and said nucleic acid including first, second, third, and fourth segments:
the first segment having a sequence that is complementary to the first primer immobilized to the solid support;
the second segment having a sequence of the target nucleic acid, or complementary to the sequence of the target nucleic acid;
the third segment having a sequence complementary to a sequencing primer; and
the fourth segment having a sequence adapted to conform into a conformation having a free energy with more favorable thermodynamics than a corresponding B-DNA duplex;
wherein binding between the first segment and the first primer results in the generation of a complementary strand to the nucleic acid;
bringing said nucleic acid and said complementary strand into contact with a second primer, wherein said second primer is adapted to conform into a conformation having a free energy with more favorable thermodynamics than a corresponding B-DNA duplex during an extension step of a reaction under isothermal conditions; and
bringing said nucleic acid and said complementary strand into contact with said sequencing primer.

24. (canceled)

25. (canceled)

26. A process for preparing a library of a target nucleic acid for sequencing by amplifying the target nucleic acid sequence, the process comprising:
bringing a nucleic acid into contact with a first primer, said first primer immobilized to a surface, wherein said first primer is adapted to conform into a conformation having a free energy with more favorable thermodynamics than a corresponding B-DNA duplex during an extension step of a reaction under isothermal conditions, and wherein said nucleic acid includes first, second, third, and fourth segments:
the first segment having a sequence that is complementary to the first primer immobilized to the solid support;
the second segment having a sequence of the target nucleic acid, or complementary to the sequence of the target nucleic acid;
the third segment having a sequence complementary to a sequencing primer; and
the fourth segment having a sequence complementary to a second primer;
wherein binding between the first segment and the first primer results in the generation of a complementary strand to the nucleic acid;
bringing said nucleic acid and said complementary strand into contact with said second primer; and
bringing said nucleic acid and said complementary strand into contact with said sequencing primer.

27. (canceled)

28. (canceled)


SEQUENCE LISTING (16 sequences)

SEQ ID NO: 1 - DNA, 15 bp, Artificial Sequence
  Sequence that can assume a dissociative conformation
gggngggtgggtggg

SEQ ID NO: 2 - DNA, 15 bp, Artificial Sequence
  Complementary sequence to a sequence that can assume a dissociative conformation
cccacccaccctccc

SEQ ID NO: 3 - DNA, 13 bp, Artificial Sequence
  Sequence that can assume a dissociative conformation
gggngggtgggtg

SEQ ID NO: 4 - DNA, 15 bp, Artificial Sequence
  Complementary sequence to a sequence that can assume a dissociative conformation
ccctcccacccaccc

SEQ ID NO: 5 - DNA, 16 bp, Artificial Sequence
  Sequence that can assume a dissociative conformation
gggtgggtgggtgggt

SEQ ID NO: 6 - DNA, 15 bp, Artificial Sequence
  Sequence that can assume a dissociative conformation
gggngggcgggcggg

SEQ ID NO: 7 - DNA, 14 bp, Artificial Sequence
  Sequence that can assume a dissociative conformation
gggngggtgggtgg

SEQ ID NO: 8 - DNA, 15 bp, Artificial Sequence
  Sequence that can assume a dissociative conformation
ggntggtgtggttgg

SEQ ID NO: 9 - DNA, 15 bp, Artificial Sequence
  Sequence that can assume a dissociative conformation
ggttggngtggttgg

SEQ ID NO: 10 - DNA, 17 bp, Artificial Sequence
  Sequence that can assume a dissociative conformation
gcgcgggtgggtgggtg

SEQ ID NO: 11 - DNA, 15 bp, Artificial Sequence
  Sequence that can assume a dissociative conformation
gggngggngggnggg

SEQ ID NO: 12 - DNA, 11 bp, Artificial Sequence
  Sequence that can assume a dissociative conformation
gggtgggtggg

SEQ ID NO: 13 - DNA, 11 bp, Artificial Sequence
  Complementary sequence to a sequence that can assume a dissociative conformation
cccacccaccc

SEQ ID NO: 14 - DNA, 15 bp, Artificial Sequence
  Sequence that can assume a dissociative conformation
gggtgggtgggtggg

SEQ ID NO: 15 - DNA, 40 bp, Artificial Sequence
  Nucleic acid construct for amplification to determine presence of a target nucleic acid
gggagggcgggtgggttttttttttttttggcccgccctc

SEQ ID NO: 16 - DNA, 20 bp, Artificial Sequence
  Target nucleic acid
ccaaaaaaaaaaaaaaccca